(19) United States
(12) Patent Application Publication
(10) Pub. No.: US 2006/0147389 A1
(43) Pub. Date: Jul. 6, 2006

(54) Devices and pharmaceutical compositions for enhancing dosing efficiency

(75) Inventors: John Nicholas Staniforth, Wiltshire (GB); David Morton, Wiltshire (GB); Michael Tobyn, Wiltshire (GB); Stephen Eason, Wiltshire (GB); Quentin Harmer, Wiltshire (GB); David Ganderton, Wiltshire (GB)

Correspondence Address: DAVIDSON, DAVIDSON & KAPPEL, LLC, 485 SEVENTH AVENUE, 14TH FLOOR, NEW YORK, NY, 10018, US

(73) Assignee: Vectura Ltd., Chippenham (GB)

(21) Appl. No.: 10/552,326

(22) PCT Filed: Apr. 14, 2004
(86) PCT No.: PCT/GB04/01628

§ 371 Date: Mar. 20, 2006

(51) Int. Cl. A61K 9/14; A61L 9/04

(52) U.S. Cl. 424/46

(57) ABSTRACT

The present invention relates to enhancing the dosing efficiency of pharmaceutical dry powder formulations administered by pulmonary inhalation. In particular, the present invention relates to the provision of dry powder inhalers and dry powder compositions which reproducibly achieve a much higher delivered dose of the pharmaceutically active agent than currently achieved.

The present invention relates to enhancing the dosing efficiency of pharmaceutical dry powder formulations administered by pulmonary inhalation. In particular, the present invention relates to the provision of dry powder inhalers and dry powder compositions which reproducibly achieve a much higher delivered dose of the pharmaceutically active agent than currently achieved.

Detailed studies of powder behaviour and performance has enabled the inventors to ascertain how to balance the various factors that affect dosing efficiency, allowing them to achieve consistent, reproducible and high delivered dose values.

The metered dose (MD) of a dry powder formulation is the total mass of active agent present in the metered form presented by the inhaler device in question. For example, the MD might be the mass of active agent present in a capsule for a Cyclohaler™, or in a foil blister in an Aspirair™ device.

The emitted dose (ED) is the total mass of the active agent emitted from the device following actuation. It does not include the material left inside or on the surfaces of the device. The ED is measured by collecting the total emitted mass from the device in an apparatus frequently identified as a dose uniformity sampling apparatus (DUSA), and recovering this by a validated quantitative wet chemical assay.

The fine particle dose (FPD) is the total mass of active agent which is emitted from the device following actuation which is present in an aerodynamic particle size smaller than a defined limit. This limit is generally taken to be 5 μm if not expressly stated to be an alternative limit, such as 3 μm or 1 μm, etc. The FPD is measured using an impactor or impinger, such as a twin stage impinger (TSI), multi-stage impinger (MSI), Andersen Cascade Impactor or a Next Generation Impactor (NGI). Each impactor or impinger has a pre-determined aerodynamic particle size collection cut points for each stage. The FPD value is obtained by interpretation of the stage-by-stage active agent recovery quantified by a validated quantitative wet chemical assay where either a simple stage cut is used to determine FPD or a more complex mathematical interpolation of the stage-by-stage deposition is used.

The fine particle fraction (FPF) is normally defined as the FPD divided by the ED and expressed as a percentage. Herein, the FPF of ED is referred to as FPF(ED) and is calculated as FPF(ED)=(FPD/ED)×100%.

The fine particle fraction (FPF) may also be defined as the FPD divided by the MD and expressed as a percentage. Herein, the FPF of MD is referred to as FPF(MD), and is calculated as FPF(MD)=(FPD/MD)×100%.

The FPF(MD) can also be termed the ‘Dose Efficiency’ and is the amount of the dose of the pharmaceutical dry powder formulation which, upon being dispensed from the delivery device, is below a specified aerodynamic particle size.

It is well known that particle impaction in the upper airways of a subject is predicted by the so-called impaction parameter. The impaction parameter is defined as the velocity of the particle times the square of its aerodynamic diameter. Consequently, the probability associated with delivery of a particle through the upper airways region to the target site of action, is related to the square of its aerodynamic diameter. Therefore, delivery to the lower airways, or the deep lung is dependent on the square of its aerodynamic diameter, and smaller aerosol particles are very much more likely to reach the target site of administration in the user and therefore able to have the desired therapeutic effect.

Particles having aerodynamic diameters in the range of 5 μm to 2 μm will generally be deposited in the respiratory bronchioles whereas smaller particles having aerodynamic diameters in the range of 3 to 0.05 μm are likely to be deposited in the alveoli. So, for example, high dose efficiency for particles targeted at the alveoli is predicted by the dose of particles below 3 μm, with the smaller particles being most likely to reach that target site.

At present, many of the commercially available dry powder inhalers exhibit very poor dosing efficiency, with sometimes as little as 10% of the active agent present in the dose actually being properly delivered to the user so that it can have a therapeutic effect. Whilst isolated incidences of high percentages of dose delivered have been possible in the prior art, it has not previously been possible to repeatedly and consistently achieve a dose efficiency at 5 or 3 μm of 70% or more.

The reason for this lack of dosing efficiency is that a proportion of the active agent in the dose of dry powder tends to be effectively lost at every stage the powder goes through from expulsion from the delivery device to deposition in the lung. For example, substantial amounts of material may remain in the device. Material may be lost in the throat of the subject due to excessive plume velocity. However, it is frequently the case that a high percentage of the dose delivered exists in particulate forms of aerodynamic diameter in excess of that required.

Therefore, the present invention provides ways in which the loss of the pharmaceutically active agent is reduced at each of these stages, so that a high dosing efficiency can be achieved.

In the past, efforts to increase dosing efficiency and to obtain greater dosing reproducibility have tended to focus on preventing the formation of agglomerates of fine active particles. Such agglomerates increase the effective size of these particles and therefore prevent them from reaching the lower respiratory tract or deep lung, where the active particles should be deposited in order to have their desired therapeutic effect.

However, it has now been recognised that other factors affect the loss of active agent during known delivery of powder formulations.

Firstly, it is common for at least some of the dose of powder formulation, including some of the active agent, to be left in the dispensing device or in the dose storage container, such as a blister or capsule after use. There are several points at which such retention in the device may occur and these will be discussed in greater detail below.

Secondly, the dynamics of the cloud of powder released by the dispensing device will affect the amount of the powder and therefore of the active agent which will become deposited in the throat of the user. Once again, active agent is effectively lost if it is deposited in the throat as it will not have any therapeutic effect. It has been found that the shape of the plume of powder formed by the device, and the velocity of the active particles in particular, will affect the deposition in the throat. This will be discussed in greater detail below.

Thirdly, as recognised in the art, the fine particles of active agent tend to agglomerate and if these agglomerates are not broken up upon actuation of the dispensing device, the active agent particles will not reach the desired part of the lung. It has been found that the deagglomeration of the fine powder particles can be greatly enhanced by the addition of force control agents which reduce particle cohesion to allow agglomerates to break up more easily, as well as by the methods used to prepare the particles.

All of the ways of improving dosing efficiency disclosed herein may be added to techniques already known and used in the art in order to achieve a dosing efficiency at 5 μm of preferably at least 65%, preferably at least 70%, preferably at least 75%, more preferably at least 80%, more preferably at least 85%, more preferably at least 90%, and most preferably at least 95%. The improvements may also lead to a dosing efficiency at 3 μm of preferably at least 60%, preferably at least 70%, more preferably at least 75%, more preferably at least 80%, more preferably at least 85%, and most preferably at least 90%. The improvements may also allow one to achieve a dosing efficiency at 2 μm of preferably at least 40%, preferably at least 50%, more preferably at least 55%, more preferably at least 60%, and most preferably at least 70%. These efficiencies are far greater than anything consistently achieved prior to this invention using simple, practical and cost effective methods of preparation, which would be suitable for the pharmaceutical industry and the methods used are described in more detail below. These methods are in stark contrast to the known technologies for producing high performance, such as the Pulmosphere technology from Nektar, or the AIR technology from Alkermese. These prior art methods use combinations of complex and expensive emulsion and spray drying techniques, including significant levels of organic solvents, and producing very low density particles.

High dosing efficiency will have a large number of benefits. For example, as it is possible to repeatedly and reliably deliver a higher proportion of the active agent in a dose, it will be possible to reduce the size of the doses whilst still achieving the same therapeutic effect. Thus, if at present a usual dose of 100 μmg of an active agent is used to achieve a desired therapeutic effect and only 10% of the active agent is being properly delivered so that it actually has a therapeutic effect, a dosing efficiency of 70% will allow the dose to be reduced to less than 15 μg whilst still achieving the same therapeutic effect! This is clearly very attractive.

Use of the techniques disclosed herein allow high levels of dose reproducibility. The reproducibility is measured in terms of relative standard deviation (RSD %) and is in the order of less than 10, less than 7.5, less than 5, less than 4 or less than 3%. Additionally, the lower dose and the high reproducibility achieved by the present invention means that the therapeutic effect achieved by a given dose will be more predictable and consistent. This obviates the risk of having an unexpected and unusually high dosing efficiency with the conventional devices and powders, which could lead to an undesirably high dose of active agent being administered, effectively an overdose.

Furthermore, high doses of therapeutically active agents has long been linked with the increased incidence of undesirable side effects. Thus, the present invention may help to reduce the incidence of side effects by reducing the dose administered to all patients.

Yet another advantage associated with the higher dosing efficiency of the present invention is that it may be possible to achieve a longer-lasting therapeutic effect without having to increase the dose administered to the patient. The greater dosing efficiency means that a greater amount of a given dose is actually delivered. This can lead to a greater therapeutic effect and, in cases where the active agent does not have a short half-life, this will also mean that the therapeutic effect lasts for a longer period of time. In some circumstances, this may even mean that it is possible to use the present invention to administer an active agent it an immediate release form and achieve the same extended therapeutic effect as a sustained release form of the same active agent.

Naturally, the reduction in the amount of an active agent required to achieve the same therapeutic effect is attractive because of the cost implications. However, it is also likely to be deemed much safer by regulatory bodies such as the FDA in the United States.

Yet another advantage associated with the reduced throat deposition, in that any unpleasant taste effects of the active will be minimised. Also, any side effects such as throat infections caused by deposition of steroids on the throat are reduced.

A particular advantage which is afforded by the high dosing efficiency achieved by the present invention is that it confirms that administration of pharmaceutically active agents in the form of a dry powder and via pulmonary inhalation is an effective and efficient mode of administration. The serum concentration of the active agent following the administration of a dry powder formulation by pulmonary inhalation according to the present invention has been shown to be consistent between doses and between different individuals. There is no variation between individuals, as is observed with other modes of administration (such as oral administration). This means that the therapeutic effect of the administration of a given dose is predictable and reliable. This has the added benefit that a balance can more easily be struck between the therapeutic effect of a pharmaceutically active agent and any adverse effects that might be associated with its administration. This will be demonstrated in one of the examples set out below.

Thus, according to a first aspect of the present invention, a dry powder dispensing device is provided with a pharmaceutical dry powder formulation, wherein the at least 70% of the dose of active agent in the dry powder is administered so as to have a therapeutic effect on the body of a patient. Preferably, the dosing efficiency remains at least 70% over numerous consecutive doses, i.e. the dosing efficiency is reproducible and constant, not an isolated good result.

This high dosing efficiency is achieved by ensuring that each stage of the dose delivery is optimised.

This requires the balancing of various factors which affect the extraction of the powder formulation from the dispensing device, the dynamics of the powder plume created by the device and the deposition of the active particles within the lung. One of the factors affecting these is the tendency of the powder particles to agglomerate. This, in turn, is linked to the size of the particles, as well as other factors, such as the presence of force controlling agents on the surface of the powder particles, particle morphology and density, and the type of device used to dispense the powder. The balancing of such factors is discussed in greater detail below. However, it is clear that the active particles and powder formulations can be tailored to the dispensing device to be used.

It must be appreciated that one cannot focus on just one particular factor affecting dose delivery, to the exclusion of all other factors. This is because the various factors affect one another and the optimisation (if possible) of one factor will not necessarily result in good dosing efficiency without the appropriate adjustment of other factors.

For example, fine particles which do not agglomerate will clearly be beneficial as all of the particles will be of the appropriate size for lung deposition. However, such powder formulations comprising such non-agglomerating particles will have poor flow characteristics, which will make extraction of the powder from the inhaler device difficult, potentially leading to loss of dosing efficiency as a result of increased device retention. If the flowability of the powder is improved, the extraction of the powder from the device is also likely to be improved. However, if the extraction of the powder becomes too easy, this can also have a detrimental effect, which is probably more marked where an active type of dry powder inhaler device is used. As a result of the improved flowability and easier extraction of the powder, it is possible that the powder will actually leave the device too quickly. This can mean that the active particles travel too quickly within the powder plume generated by the device and these particles therefore tend to impact on the subject's throat rather than being inhaled. Thus, the dosing efficiency is once again reduced, this time as a result of increased throat impaction or deposition.

In a preferred embodiment of the present invention, the amount of active agent retained in the blister or capsule following actuation of the device is less than 15%, preferably less than 10%, more preferably less than 7% and most preferably less than 5% or 3%.

In another preferred embodiment, the amount of the powder formulation retained in the dispensing device, for example in the blister or capsule, in the mouthpiece and in any vortex chamber or equivalent device part, is less than 15%, preferably less than 10%, more preferably less than 7% and most preferably less than 5% or 3%.

In a yet further embodiment, upon being expelled from the dispensing device, the powder formulation has a dosing efficiency at 5 μm of preferably at least 70%, preferably at least 75%, more preferably at least 80%, more preferably at least 85%, more preferably at least 90%, and most preferably at least 95%.

Preferably, upon being expelled from the dispensing device, the powder formulation has a dosing efficiency at 3 μm of preferably at least 60%, preferably at least 70%, more preferably at least 75%, more preferably at least 80%, more preferably at least 85%, and most preferably at least 90%.

Preferably, upon being expelled from the dispensing device, the powder formulation has a dosing efficiency at 2 μm of preferably at least 40%, preferably at least 50%, more preferably at least 55%, more preferably at least 60%, and most preferably at least 70%. These efficiencies are far greater than anything consistently achieved prior to this invention.

In another preferred embodiment, the particles comprising a pharmaceutically active agent (active particles) have a mass median aerodynamic diameter (MMAD) of less than 10 μm. Preferably the MMAD of the active particles is less than 7 μm, more preferably less than 5 μm, more preferably less than 2 μm, and most preferably less than 1.51 μm.

Finally, in another preferred embodiment, the amount of the active agent which is deposited in the throat of the user is less than 15% of the active agent in the metered dose. Preferably, throat deposition is less than 10%, more preferably it is less than 7% and most preferably it is less than 5% or less than 3%.

The foregoing powder retention, FPF, MMAD and throat deposition figures may be achieved by adopting one or more of the following adaptations to conventional dry powder dispensing devices, dry powder formulations or methods for preparing dry powder formulations. Combinations of these will lead to a dose delivery of at least 70%.

Preferred embodiments of the invention will now be described in detail in the following sections of this specification. These embodiments represent various separate means of putting the present invention into effect. These embodiments may be used separately or in combination. When used in combination, the embodiments described in the following sections will provide enhanced results in terms of dosing efficiency and dose reproducibility.

Reproducibility is very important in the present invention. The unpredictable nature of the conventional powder systems means that doses they provide can vary significantly. Given that the dosing is usually inefficient, the amount of active agent in a dose is generally much higher than is to actually be administered to the subject. However, the variable efficiency of the dosing can result in too much active agent being administered and this may be the cause of adverse side effects in some instances. Alternatively, the dosing may be less efficient than predicted, leading to an ineffective dose being administered so that the desired therapeutic effect is not achieved.

Where, the dosing is unpredictable, it is possible for conventional powder systems to provide high dosing efficiency on a one-off basis. However, these conventional powder systems will not provide high dosing efficiency on a consistent or repeatable and predictable basis, as the powder systems according to the present invention do.

The present invention can be carried out with any pharmaceutically active agent. The preferred active agents include:

1) steroid drugs such as, for example, alcometasone, beclomethasone, beclomethasone dipropionate, betamethasone, budesonide, clobetasol, deflazacort, diflucortolone, desoxymethasone, dexamethasone, fludrocortisone, flunisolide, fluocinolone, fluometholone, fluticasone, fluticasone proprionate, hydrocortisone, triamcinolone, nandrolone decanoate, neomycin sulphate, rimexolone, methylprednisolone and prednisolone;

2) antibiotic and antibacterial agents such as, for example, metronidazole, sulphadiazine, triclosan, neomycin, amoxicillin, amphotericin, clindamycin, aclarubicin, dactinomycin, nystatin, mupirocin and chlorhexidine;

3) systemically active drugs such as, for example, isosorbide dinitrate, isosorbide mononitrate, apomorphine and nicotine;

4) antihistamines such as, for example, azelastine, chlorpheniramine, astemizole, cetirizine, cinnarizine, desloratadine, loratadine, hydroxyzine, diphenhydramine, fexofenadine, ketotifen, promethazine, trimeprazine and terfenadine;

5) anti-inflammatory agents such as, for example, piroxicam, nedocromil, benzydamine, diclofenac sodium, ketoprofen, ibuprofen, heparinoid, nedocromil, cromoglycate, fasafungine and iodoxamide;

6) anticholinergic agents such as, for example, atropine, benzatropine, biperiden, cyclopentolate, oxybutinin, orphenadine hydrochloride, glycopyrronium, glycopyrrolate, procyclidine, propantheline, propiverine, tiotropium, tropicamide, trospium, ipratropium bromide and oxitroprium bromide;

7) anti-emetics such as, for example, bestahistine, dolasetron, nabilone, prochlorperazine, ondansetron, trifluoperazine, tropisetron, domperidone, hyoscine, cinnarizine, metocelopramide; cylizine, dimenhydrinate and promethazine;

8) hormonal drugs such as, for example, protirelin, thyroxine, salcotonin, somatropin, tetracosactide, vasopressin or desmopressin;

9) bronchodilators, such as salbutamol, fenoterol and salmeterol;

10) sympathomimetic drugs, such as adrenaline, noradrenaline, dexamfetamine, dipirefin, dobutamine, dopexamine, phenylephrine, isoprenaline, dopamine, pseudoephedrine, tramazoline and xylometazoline;

11) anti-fungal drugs such as, for example, amphotericin, caspofungin, clotrimazole, econazole nitrate, fluconazole, ketoconazole, nystatin, itraconazole, terbinafine, voriconazole and miconazole;

12) local anaesthetics such as, for example, amethocaine, bupivacaine, hydrocortisone, methylprednisolone, prilocaine, proxymetacaine, ropivacaine, tyrothricin, benzocaine and lignocaine;

13) opiates, preferably for pain management, such as, for example, buprenorphine, dextromoramide, diamorphine, codeine phosphate, dextropropoxyphene, dihydrocodeine, papaveretum, pholcodeine, loperamide, fentanyl, methadone, morphine, oxycodone, phenazocine, pethidine and combinations thereof with an anti-emetic;

14) analgesics and drugs for treating migraine such as clonidine, codine, coproxamol, dextropropoxypene, ergotarnine, sumatriptan, tramadol and non-steroidal anti-inflammatory drugs;

15) narcotic agonists and opiate antidotes such as naloxone, and pentazocine;

16) phosphodiesterase type 5 inhibitors, such as sildenafil; and

17) pharmaceutically acceptable salts of any of the foregoing.

A plurality of active agents can be employed in the practice of the present invention.

In preferred embodiments, the active agent is heparin, apomorphine, glycopyrrolate, clomipramine or clobozam.

Delivery Devices

The device used to deliver the dry powder formulations is clearly going affect the performance of the dry powder formulations and the device is therefore a very important part of present invention.

Dry powder inhaler devices (DPIs) are well known in the art and there are a variety of different types. Generally, the dry powder is stored within the device and is extracted from the place of storage upon actuation of the device, whereupon the powder is expelled from the device in the form of a plume of powder which is to be inhaled by the subject. In most DPIs, the powder is stored in a unitary manner, for example in blisters or capsules containing a predetermined amount of the dry powder formulation. Some DPIs have a powder reservoir and doses of the powder are measured out within the device. These reservoir devices are less favoured in the present invention as the blisters or capsules tend to provide more accurate doses.

As briefly discussed above, there are a number of factors associated with the delivery devices which will affect the dosing efficiency achieved. Firstly, there is the extraction of the dose. Additionally, the dynamics of the powder plume generated will also affect dosing delivery.

The dry powder inhaler devices suitable for use in the present invention include “single dose” devices, for example the Rotahaler™, the Spinhaler™ and the Diskhaler™ in which individual doses of the powder composition are introduced into the device in, for example, single dose capsules or blisters, and also multiple dose devices, for example the Turbohaler™ in which, on actuation of the inhaler, one dose of the powder is removed from a reservoir of the powder material contained in the device.

Dry powder inhalers can be “passive” devices in which the patient's breath is the only source of gas which provides a motive force in the device. Examples of “passive” dry powder inhaler devices include the Rotahaler and Diskhaler (GlaxoSmithKline) and the Turbohaler (Astra-Draco) and Novolizer™ (Viatris GmbH). Alternatively, “active” devices may be used, in which a source of compressed gas or alternative energy source is used. Examples of suitable active devices in Aspirair™ (Vectura Ltd) and the active inhaler device produced by Nektar Therapeutics.

Particularly preferred “active” dry powder inhalers are described in more detail in WO 01/00262, WO 02/07805, WO 02/89880 and WO 02/89881, the contents of which are hereby incorporated by reference. It should be appreciated, however, that the compositions of the present invention can be administered with either passive or active inhaler devices.

According to an embodiment of the present invention, an active inhaler device may be used to dispense the apomorphine dry powder formulations, in order to ensure that the best fine particle fraction and fine particle dose is achieved and, very importantly, that this is achieved consistently. Preferably, the inhaler device includes a breath triggering means such that the delivery of the dose is triggered by the onset of the patient's inhalation. This means that the patient does not need to coordinate their inhalation with the actuation of the inhaler device and that the dose can be delivered at the optimum point in the inspiratory flow. Such devices are commonly referred to as “breath actuated”.

As already mentioned, in the case of certain powders, an active inhaler device offers advantages in that a higher fine particle fraction and a more consistent dose to dose repeatability will be obtainable than if other forms of device were used. Such devices include, for example, the Aspirair™ or the Nektat Therapeutics active inhaler device, and may be breath actuated devices of the kind in which generation of an aerosolised cloud of powder is driven by inhalation of the patient.

Dose Extraction

It is common for dry powder formulations to be pre-packaged in individual doses, usually in the form of capsules or blisters which each contain a single dose of the powder. In such devices, the doses will be accurately measured and consistent.

However, it is also known for powders to be held in a reservoir in a dispensing device. In such a case, a predetermined amount of powder is measured out and then dispensed by the device. Inevitably, such an arrangement will allow for some variation in the size of the dose between actuations of the same device. This will especially be the case where the amount of powder to be dispensed is relatively small, as it is difficult to accurately measure out small amounts of dry powder in such devices. Therefore, as the present invention is concerned with dose accuracy and reproducibility, devices which hold the dry powder to be dispensed in a reservoir are not preferred.

Actuation of the dispensing device refers to the process during which a dose of the dry powder formulation is removed from its rest position in the inhaler (be it in a blister or capsule or other container). The actuation may be caused by the user of the device inhaling in the case of a passive device, or by firing an active device. The actuation of a dispensing device occurs after the powder has been loaded ready for use within the device.

Improved Evacuation of Dose from Packaging

As already mentioned above, it is common for some of the dose to be deposited in the inhaler when it is used or, for some of the dose to remain in the pack in which the dose is stored. Reference will now be made to embodiments of the invention which seek to minimise the deposition of the dose on the inhaler and the retention of dose within the pack.

It will be appreciated that an important factor in maintaining the efficiency, accuracy and repeatability of the dose is to minimise the amount of drug that is retained in the inhaler mechanism and in the medicament pack in which the drug is stored prior to inhalation using the device. A conventional pack for an individual dose of dry powder medicament may include a gelatin capsule or a foil blister which is cold formed from a ductile foil laminate. A piercable foil laminate lid usually covers the blister which is heat sealed around the periphery of the blister. These types of package are preferred because each dose is protected from the ingress of water and penetration of gases such as oxygen in addition to being shielded from light and UV radiation and so offer excellent environmental protection. To administer a dose using a compressed gas powered inhaler, the capsule or foil lid is punctured by a piercing mechanism so that the drug can be entrained and carried to an aerosolising means, such as a nozzle, in a charge of gas which passes through the capsule or blister to the nozzle.

In an active inhaler of the aforementioned type, the same charge of gas provides the energy needed for both entraining the drug to evacuate the packaging and for aerosolising the drug once it has reached the nozzle. It is therefore important that the primary packaging does not present a significant restriction to the gas flow from the source of pressurised gas to the aerosolising nozzle. Bearing in mind that the amount of gas available for each dose is limited by what can be stored in a pressurised canister or generated in the device by the user by, for example, using a manually operated pump, the efficiency by which the drug is entrained in the airflow and so evacuated from its packaging must be as high as possible.

As mentioned above, a problem with known inhalation devices is that it is possible for not all of the drug to be entrained in the airflow each time the device is used because the blister or capsule, in which the dose is stored, is typically pierced in such a way that the gas flowing into the blister through the pierced foil only partially scours the blister surfaces before flowing out of the blister. This problem is often exacerbated by the flap of foil cut by the piercing element as this can obscure parts of the blister from the flow of gas thereby restricting the free flow of gas throughout the entire volume of the blister and creating “dead” regions where gas flow is minimal or where secondary eddies form leading to powder becoming trapped. This trapped powder will have a significant detrimental effect on the repeatablility and accuracy of the delivered dose as well as on the overall efficiency of the inhaler.

This aspect of the invention seeks to provide a dry powder inhaler in which all, or substantially all, of the internal surfaces of a pack containing a medicament dose are swept by the airflow so that substantially all of the drug is evacuated from the pack for delivery through an aerosolising nozzle and out of the device into the airway of a patient, thereby improving the delivered dose and hence the fine particle fraction of the delivered dose.

Accordingly, there is provided a dry powder inhaler for delivering a dose of medicament for inhalation by a user, the dose being contained in a medicament pack having a puncturable lid, the inhaler comprising a drug entrainment device including a drug outlet tube terminating with a primary piercing element to pierce an opening in said lid when a pack is located in the inhaler, a secondary piercing member to pierce a plurality of peripheral openings in said lid and, an airflow path to enable the supply of a charge of gas into the pack via said peripheral openings to scour the interior of a pierced pack such that substantially all of the dose is entrained in the gas and flows out of the pack via the drug outlet tube.

Preferably, the drug entrainment device includes an airflow inlet for the flow of air from the airflow path into a plenum chamber formed above the pierced lid of a pack, the inlet and the plenum chamber being configured such that a swirling airflow is generated in the plenum chamber.

In a preferred embodiment, the plenum chamber is substantially cylindrical in shape and the inlet intersects the curved wall of the chamber at a tangent thereto.

The secondary piercing member is preferably configured to direct the swirling flow of air in the plenum chamber into the pack through the openings formed therein by the secondary piercing member. Advantageously, the secondary piercing member comprises a plurality of blades with a vane depending from each blade for piercing the lid of the pack and to direct the swirling airflow into the pack. This introduces swirl into the blister to improve the entrainment of the dose by ensuring that the surfaces of the blister are swept by the gas flow. The generation of swirl in the blister or capsule containing the drug also reduces the speed of delivery of the drug to the aerosolising nozzle and therefore assists in reducing the likelihood of deposition of drug in the aerosolising nozzle. The maximum loading of powder passing through the nozzle must be kept below a threshold otherwise the nozzle can become overloaded and its efficiency reduces. If the dose is introduced over a longer period of time, the powder density in the nozzle is kept sufficiently low and its efficiency is maintained.

Many drug formulations suitable for inhalation are highly cohesive and tend to adhere to the internal surfaces of the inhaler. Therefore, in addition to evacuating the primary packaging efficiently, it is also equally important to prevent deposition of the drug on the internal parts of the inhaler once it has been entrained in the airflow and whilst it travels through the aerosolising nozzle and mouthpiece into a users airway as this can also have a detrimental effect on the delivered dose. Furthermore, deposited drug may become detached during subsequent use of the inhaler resulting in an unpredictable variation in the delivered dose. Although this problem is partially alleviated because each dose is individually packaged so that any drug remaining in a used primary package is removed and disposed of together with that primary package and so cannot have any effect on the delivered dose during subsequent uses of the inhaler, any residual drug remaining in unwiped or inaccessible parts of the inhaler can still have an appreciable effect on the delivered dose and in subsequent uses of the inhaler. Although the passage from the primary packaging to the nozzle does not present a significant restriction to the gas flow and hence regions where deposition may easily occur, the aerosolising nozzle is particularly susceptible to deposition as the medicament entrained in the airflow enters the nozzle at high speed and over a very short period of time resulting in a proportion of the powdered medicament adhering to the walls of the nozzle.

The present aspect of the invention also seeks to overcome or substantially alleviate the aforementioned problem caused by residual drug remaining in the nozzle and in the flow path between the primary package and the nozzle during subsequent inhalations which can have a detrimental effect on the delivered dose of medicament and the fine particle fraction of the delivered dose.

Accordingly, there is also provided a medicament pack for use in an inhalation device comprising a drug storage chamber to contain a single dose of medicament and an aerosolising nozzle for generating an inhalable aerosol of the dose for inhalation by a user when a charge of gas is passed through the pack. Preferably the pack, incorporating both the drug storage chamber and the nozzle is disposed of after the drug has been discharged therefrom and is not re-filled.

In a preferred embodiment, the drug storage chamber and the aerosolising nozzle are integrally formed into a single module.

In one embodiment, the medicament pack comprises a blister having two compartments forming the drug storage chamber and the aerosolising nozzle respectively, each compartment being sealed with a piercable lid to enable an inhaler to pierce an inlet for the gas in the dose storage chamber and an outlet for the aerosolised dose in the aerosolising nozzle.

Preferably, an integral drug feed path communicates the drug storage chamber with the aerosolising nozzle.

In another embodiment, the drug storage compartment and the aerosolising nozzle are integrally formed from a moulded plastics material which is sealed with a piercable lid to enable an inhaler to pierce an inlet for the flow of gas into the dose storage chamber and an outlet for aerosolised dose in the aerosolising nozzle.

Alternatively, the drug storage compartment and the aerosolising nozzle are integrally formed from a moulded plastics material which is sealed with a piercable lid to enable an inhaler to pierce an inlet for the flow of gas into the drug storage chamber, an aperture being formed in the moulded plastic to form an outlet for the dose from the aerosolising nozzle.

In another embodiment, the medicament pack comprises a sheet in which is formed a plurality of drug storage chamber and nozzle pairs. Alternatively, a single nozzle and a plurality of drug storage chambers can be formed in the sheet, a drug feed path connecting each of the drug storage chambers with the nozzle.

In a preferred embodiment, the nozzle is a substantially cylindrical vortex chamber. The inlet from the drug feed tube intersects the chamber at a tangent and the outlet is coaxial with the longitudinal axis of the cylinder. The cylinder may be provided with a frustoconical portion in the region of the outlet for directing the airflow within the chamber towards the outlet.

Embodiments of this aspect of the invention will now be described, by way of example only, with reference to FIGS. 2 to 11 of the accompanying drawings, in which:

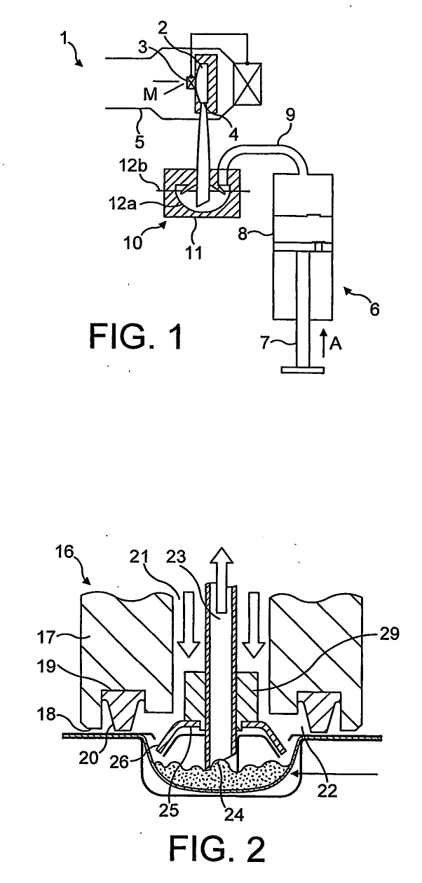

FIG. 1 represents a schematic diagram of a conventional pressurised gas powered active dry powder inhaler;

FIG. 2 shows a cross-sectional side elevation of a portion of a drug entrainment device according to the invention, after piercing of a blister has taken place, for use in the pressurised gas powered inhaler of FIG. 1;

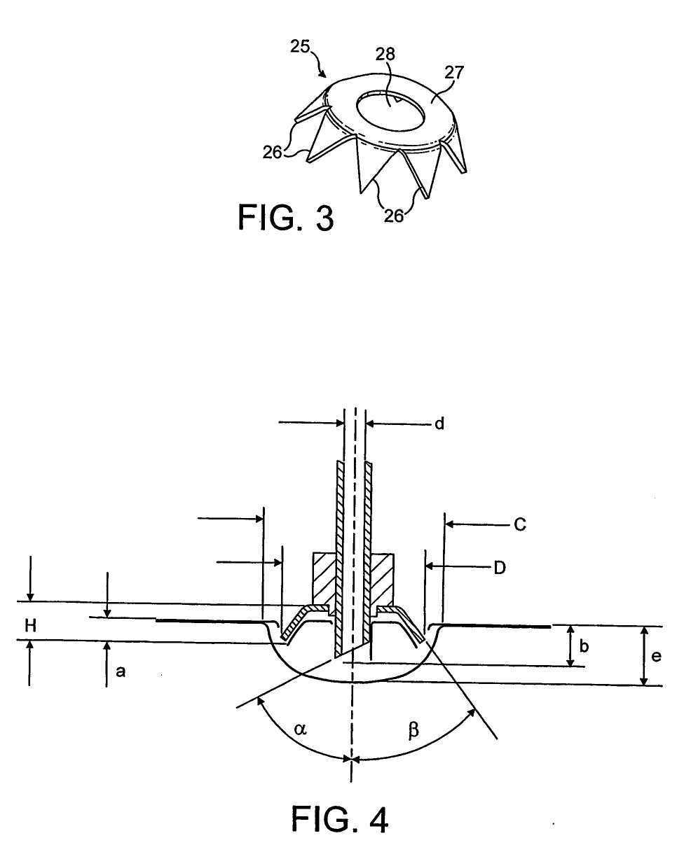

FIG. 3 illustrates a perspective view of the secondary piercing element used in the drug entrainment device shown in FIG. 2;

FIG. 4 shows a cross-sectional side elevation of a portion of the drug entrainment device of FIG. 2;

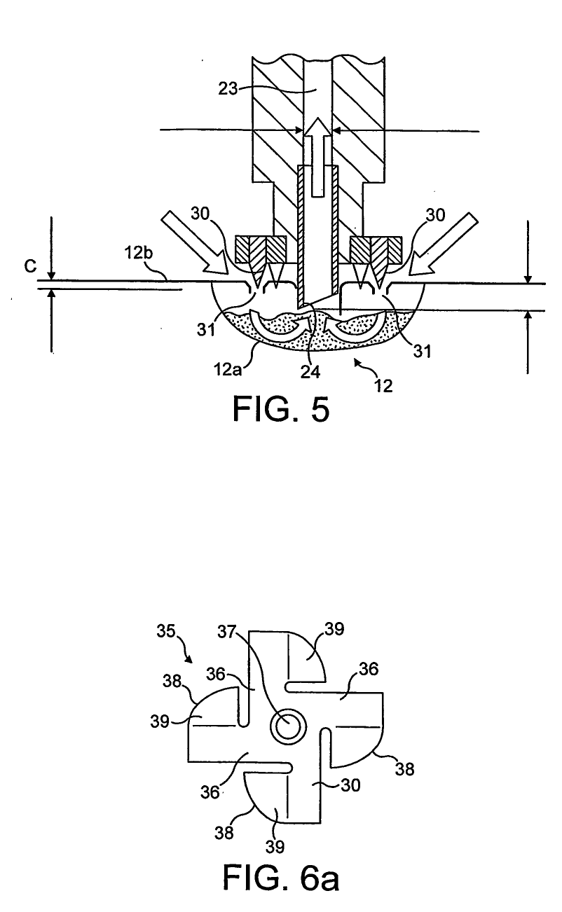

FIG. 5 illustrates an alternative embodiment of the drug entrainment device shown in FIG. 2;

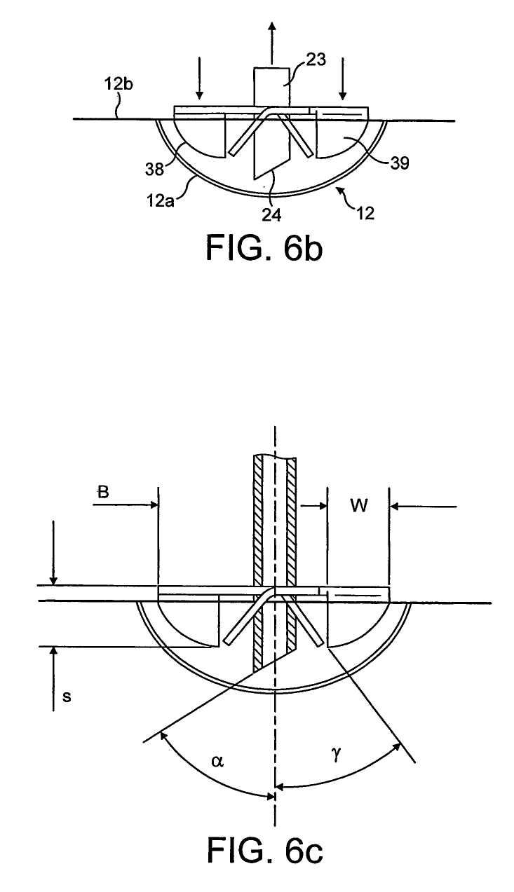

FIGS. 6A, 6B and 6C illustrate top plan and side views respectively, of an alternative version of secondary piercing element which serves to impart a swirling motion to the airflow as it passes into and through the blister;

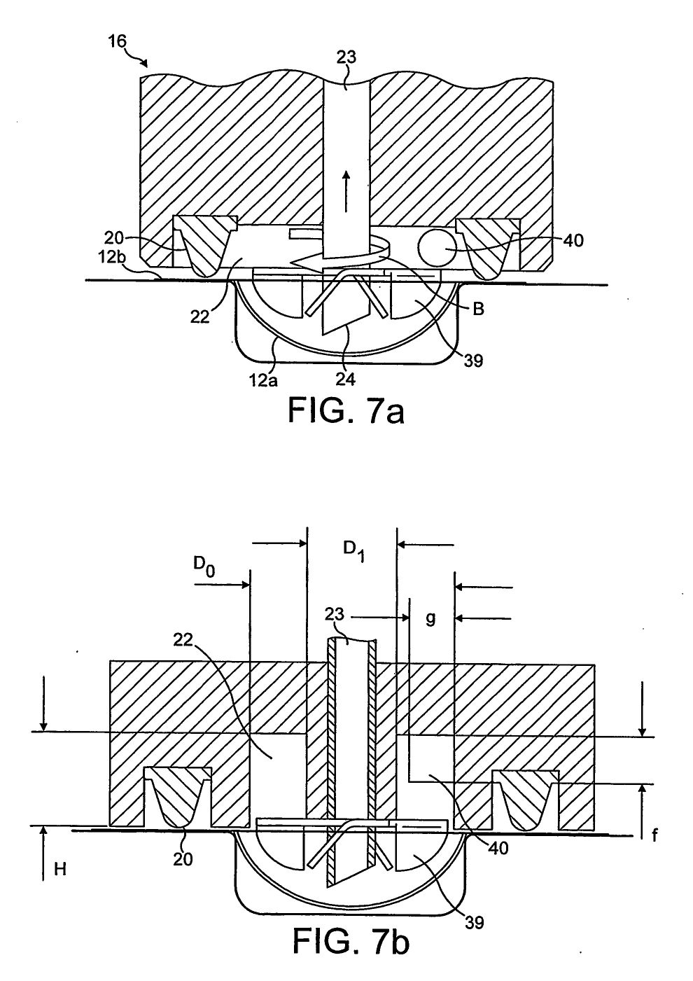

FIGS. 7A and 7B illustrate two cross-sectional side elevations of a modified version of the drug entrainment device shown in FIG. 2, using the secondary piercing element of FIGS. 6A and 6B;

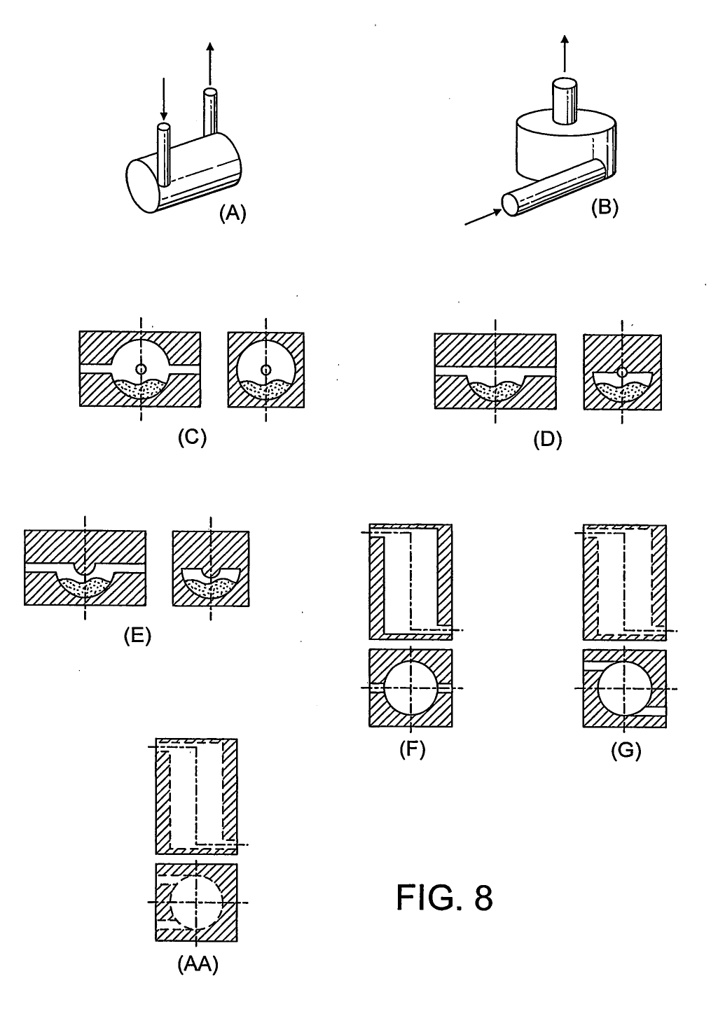

FIG. 8A to 8G illustrate various versions of medicament packs which promote the entrainment and evacuation of the dose therefrom;

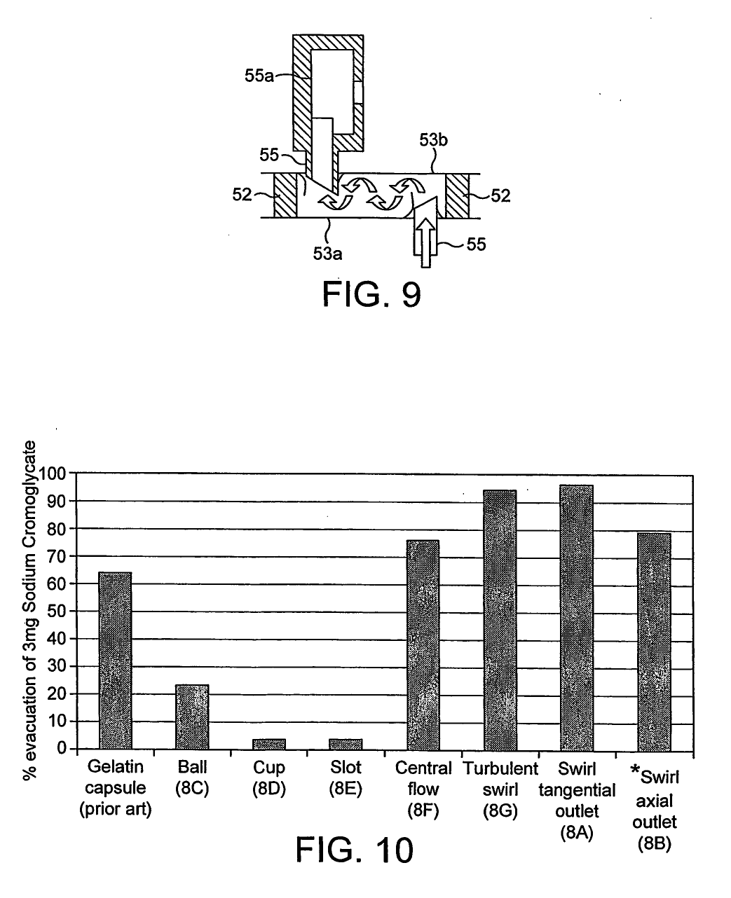

FIG. 9 illustrates another embodiment of blister pack for containing a dose of medicament for use in an inhaler;

FIG. 10 is a table to illustrate the performance of some of the medicament packs shown in FIGS. 8A to 8G, and

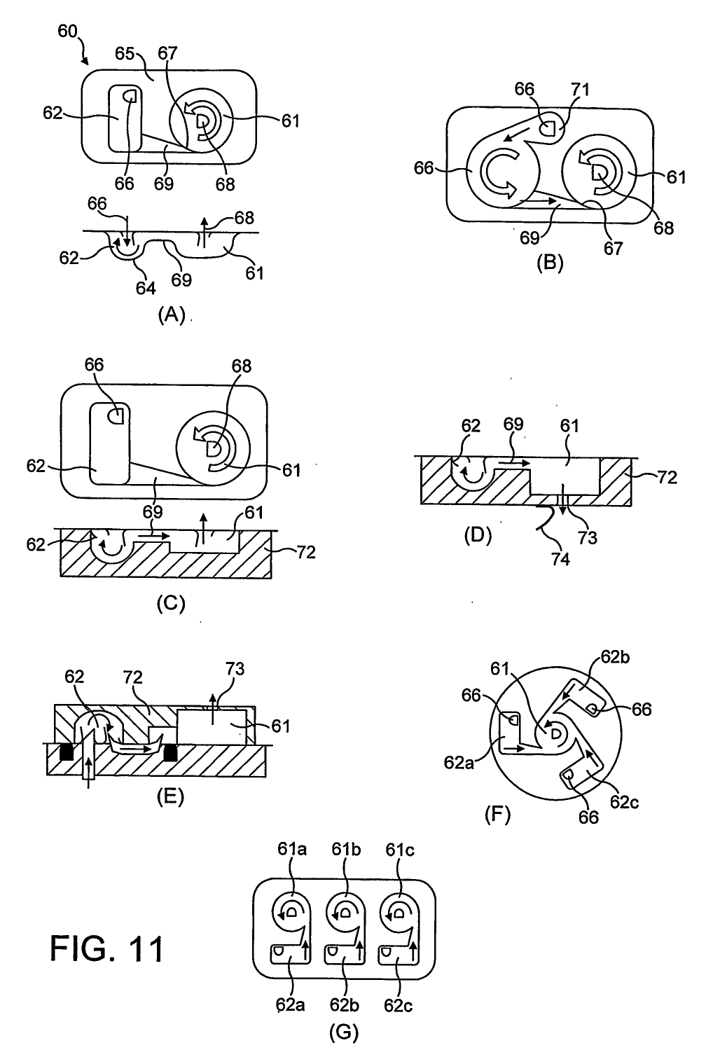

FIG. 11A to 11G illustrate various medicament packs incorporating a aerosolising nozzle according to the invention.

Referring now to the prior art drawing of FIG. 1, a pressurised gas powered active dry powder inhaler 1 for aerosolising a powdered medicament for inhalation by a user is shown. The inhaler 1 comprises a vortex chamber or nozzle 2 having an exit port 3 and an inlet port 4 for generating an aerosol of medicament M. The nozzle 2 is located within a mouthpiece 5 through which a user inhales the aerosolised medicament M.

The powdered medicament or drug M is supplied to the nozzle 2 in a gas or airflow generated by a pump represented in FIG. 1 as a piston pump 6 comprising a plunger 7 received in a pump cylinder 8 and a reservoir fluidly connected to the pump via a non-return valve. An airflow path 9 extends from the pump cylinder 8 to a drug entrainment device 10 disposed above a housing 11 to support a foil blister 12 containing a single dose of medicament (typically between 0.5 and 5 mg). The blister 12 has a cold formed foil blister base 12a sealed with a hard rolled foil laminate lid 12b chosen to facilitate piercing. A drug feed tube 13 extends from the inlet port 4 of the nozzle 2 and into the housing 11 where it terminates in a piercing element 14. When the inhaler 1 is to be used, the reservoir is primed with a charge of compressed air by sliding the plunger 7 into the pump cylinder 8 (in the direction of arrow “A” in FIG. 1 to compress the air contained therein. Thereafter, the housing 11 and the drug feed tube 13 are moved relative to each other to cause the piercing element 14 to break the foil laminate layer 12b and penetrate into the blister 12 so that when the user inhales through the mouthpiece, a valve, which may be breath actuated, releases the charge of compressed air from the reservoir so that it flows along the airflow path 9 through the blister 12 where it entrains the medicament contained therein. The airflow together with the entrained drug flows up through the drug feed tube 13 and into the nozzle 2 via the inlet 4 where a rotating vortex of medicament and air is created between the inlet and outlet ports 4,3. As the medicament passes through the nozzle 2, it is aerosolised by the high turbulent shear forces present in the boundary layer adjacent thereto as well as by the high level of turbulence in the vortex chamber and through collisions between agglomerates and other agglomerates and between agglomerates and the walls of the nozzle. The aerosolised particles exit the nozzle 2 via the exit port 3 and are inhaled by the user through the mouthpiece 5.

FIG. 2 illustrates part of a drug entrainment device 16 suitable for use with the conventional dry powder inhaler 1 illustrated in FIG. 1. The drug entrainment device 16 improves access to the medicament contained in a blister 12 and ensures that its internal surface is swept and scoured by the airflow so that all or substantially all of the medicament (at least 95%) is entrained in the airflow and carried to the aerosolising nozzle thereby increasing the delivered dose and reducing the respirable dose variation between successive uses of the inhaler.

Prior to use, the blister 12 is inserted into the housing 11 within the inhaler 1 so that its piercable lid 12b is located below the drug entrainment device 16. The drug entrainment device 16 comprises a body 17 having a lower end 18 in which is formed a channel 19 to receive a sealing member 20 which makes contact with the blister 12 around the periphery of the laminate lid 12b so as to form a fluid tight seal therewith. An annular conduit 21 extends through the drug entrainment device 16 via a plurality of holes which join and widen at their lower end 18 in the vicinity of the sealing member 20 so as to form a plenum chamber 22 above the blister lid 12b when the sealing member 19 is in sealing engagement with the periphery thereof. The opposite unillustrated end of the annular conduit 21 is connected via a valve to a source of pressurised gas such as a piston pump 6 as described with reference to FIG. 1. A central drug feed tube 23 extends axially through the annular conduit 21 and protrudes beyond the lower end 18 and the sealing member 20 and terminates in an angled face to form a central piercing element 24 for cutting the lid 12b of the blister 12. A secondary peripheral piercing member 25 is mounted on the central drug feed tube 23 adjacent to the angled end forming the central piercing element 24 for making multiple additional piercings in the surface of the blister lid 12b for reasons that will become apparent. The opposite end of the drug feed tube 25 is in communication with an aerosolising nozzle such as the nozzle 2 described with reference to the inhaler of FIG. 1.

A perspective view of the secondary piercing member 25 is shown in FIG. 3 from which it will be appreciated that it comprises a star shaped ring incorporating a plurality of peripheral pointed piercing elements 26 which are deflected or angled out of the plane of the body 27 of the ring. In the illustrated embodiment, there are eight pointed piercing elements 26. However, it has been found that the improved drug entrainment provided by the invention is achieved with 4 piercing elements 26, although 8 piercing elements 26 have been found to provide the most significant advantages. An aperture 28 in the centre of the body 27 is dimensioned so as to engage with a mounting member 29 fixedly attached to the lower end of the outer surface of the drug feed tube 23 so that the pointed piercing elements 26 point in the same direction as the central piercing element 24 and towards the lid 12b of a blister 12 mounted in the housing 11 prior to use.

The secondary piercing member 25 is preferably manufactured by chemical milling from stainless steel sheet and subsequent pressing. A further advantageous embodiment for high volume manufacture is to integrate the primary and secondary piercing members 24, 25 in a single injection moulded part. Possible materials include polyetheretherketone (PEEK), liquid crystal polymer (LCP), polyamide, polysulphone (PS), polyetherimide (PEI), polyphenylsulphone (PPS) and thermosetting plastics.

When the device is used, a blister 12 is inserted into the housing 11 and is brought up to meet the drug entrainment device 16 such that the central piercing element 24 and each of the secondary piercing elements 26 pierce the foil lid 12b and thereby create a pattern of openings in the surface of the blister 12b. When the valve (not shown) between the source of compressed air and the annular conduit 21 is opened, possibly in response to the user's inhalation, a charge of pressurised gas flows down through the annular conduit 21 and into the plenum chamber 22 and from there through the multiple piercings in the lid 12b formed by the secondary piercing elements 26 into the blister 12 so that the medicament is entrained in the airflow and flows up the drug feed tube 23 to the aerosolising nozzle.

It has been found that by using the aforementioned combination of central piercing element 24 and secondary peripheral piercing elements 26, the airflow through the blister is significantly improved so that nearly all of the medicament is entrained and evacuated from the blister 12 without any powder becoming trapped in spaces that have not been swept or scoured by the airflow. As a result, the delivered dose of medicament is improved, as is the fine particle fraction of total dose. It will be appreciated that the secondary piercing elements 26 create a smoothly controlled and predictable cut as the tip of each secondary piercing element 26 first creates a hole in the foil laminate 12a and “pushes” the cut foil flap out of the way. This should be contrasted with conventional pin type piercing elements which effectively burst through and tear the foil laminate forming unpredictable cut edges and flaps which can have a detrimental effect on the airflow through the blister 12. Furthermore, the secondary piercing elements 26 act as baffles to prevent the airflow entering the blister 12 from passing straight through it from the openings made by the secondary piercing elements 26 to the outlet feed tube 23. It should also be noted that the charge of compressed gas flows directly into and through the blister rather than being used to induce a secondary flow of air through the blister. By allowing the charge of compressed gas to pass directly through the blister entrainment of the medicament is significantly more efficient.

The inventors have also found that a number of factors have a significant influence on the amount of drug that is consistently evacuated from the blister during repeated use of the device. In particular, the shape, angle number and configuration of the secondary piercing elements 26 has a significant effect on the airflow through the blister 12, as does the diameter of the outlet feed tube 23 and its depth of penetration into the blister 12. To explain these factors in more detail, reference will be made to FIG. 4 and Tables 1 to 3.

A number of tests were conducted. These tests were part of a fractional factorial design experiment in which 10 variables were evaluated. A 3 mg dose of pure micronised sodium cromoglycate was used with a reservoir of 15 ml of air at a pressure of 1.5 bar gauge. The dose was contained in a foil blister of the type described and having the dimensions referred to in Table 3 with reference to FIG. 4. All the variables together with the preferred ranges, most preferred ranges and preferred values are shown in Table 3, which should be considered in conjunction with the drawing of FIG. 3.

Considering first the drug feed tube 23, Table 1 shows the results of evacuation from the blister 12 using a drug feed tube 23 having a first internal diameter (“d” in FIG. 4) of 1.50 mm and another drug feed tube 23 having a second internal diameter “d” of 1.22 mm. It can be ascertained from Table 1 that both the average evacuation and the repeatability of evacuation are better with a 1.22 mm diameter outlet tube than with a 1.5 mm diameter feed tube 23. As can be seen from Table 3, it was found that 1.22 mm was the most preferred value for the internal diameter of the drug feed tube 23.

TABLE 1
Blister evacuation with different outlet tube diameters

| | Average | Average standard |
|---|---|---|
| | evacuation over | deviation of evacuation |
| | four sets of 10 tests | for four sets of 10 tests |
| Outlet tube internal | 80.0 | 7.5 |
| diameter (d) = 1.50 mm | | |
| Outlet tube internal | 96.4 | 2.0 |
| diameter (d) = 1.22 mm | | |

Referring now to Table 2, this shows the effect on the evacuation from a blister 12 when the distance by which the drug feed tube 23 protrudes into the blister 12 (“b” in FIG. 4) is altered. In the first test, the drug feed tube 23 is positioned so as to protrude into blister 12 by 2.1 mm and in a second test, the drug feed tube 23 is allowed to protrude into the blister 12 by a distance of 2.4 mm. The results show that evacuation from the blister 12 is improved if the drug feed tube 23 protrudes less far into the blister 12. As can be seen from Table 3, it was found that 1.6 mm was the most preferred value for the depth of penetration of the drug feed tube 23 into the blister 12. However, it was found that penetration depths in the range 1.5 to 2.7 mm produced satisfactory results although a range of between 1.5 to 1.9 mm is largely preferred.

TABLE 2
Blister evacuation when the protrusion of the outlet tube is
at two different settings

| | Average % | Average standard |
|---|---|---|
| | evacuation over | deviation of evacuation |
| | four sets of 10 tests | for four sets of 10 tests |
| Protrusion of outlet tube | 96.7 | 1.9 |
| into blister (b) = 2.1 mm | | |
| Protrusion of outlet tube | 79.6 | 7.6 |
| into blister (b) = 2.4 mm | | |

The evacuation quoted in FIGS. 6 to 9 was measured as follows: sodium cromoglycate was weighed into an empty foil blister using a five figure balance and the fill weight recorded. The blister was then tested in an Aspirair device (described in the Applicant's earlier published PCT application No. WO 01/00262) delivering a reservoir of 10 ml of air at a pressure of 1.5 bar. The blister was then re-weighed and the new weight recorded (as evacuated weight). The evacuation efficiency of the entrainment device was calculated using the following formula:

$$Evacuation = \frac{Fill Weight - Evacuated Weight}{Fill Weight} \times 100$$

As mentioned above, Table 3 lists all the additional factors that affect the evacuation of the drug from the blister 12 with particular reference to the dimensions and shape of the secondary piercing elements 26.

TABLE 3
Preferred dimensions for the secondary piercing member of FIG. 3

| | | | Most | | | |
|---|---|---|---|---|---|---|
| | Preferred | | preferred | | Most preferred | |
| Feature | range | | range | | value | |
| Inscribing diameter, D of the | 4-9 | mm | 5-7 | mm | 6.8 | mm |
| secondary piercing elements | | | | | | |
| Height of secondary piercing | 1.2-2.0 | mm | 1.4-1.8 | mm | 1.6 | mm |
| member, H | | | | | | |
| Internal diameter, d, of the | 1.0-1.5 | mm | 1.20-1.30 | mm | 1.22 | mm |
| outlet tube | | | | | | |
| Number of secondary | 4-10 | | 6-8 | | 8 | |
| piercing elements | | | | | | |
| Protrusion, a, of the | 0.9-2.0 | mm | 1.1-1.5 | mm | 1.20 | mm |
| secondary piercing member | | | | | | |
| into the blister | | | | | | |
| Protrusion, b, of the outlet | 1.5-2.7 | mm | 1.5-1.9 | mm | 1.6 | mm |
| tube into the blister | | | | | | |
| Angle, α, of the face of the | 30-70 | degrees | 45-70 | degrees | 60 | degrees |
| outlet tube to its axis | | | | | | |
| Angle, β, of the secondary | 30-60 | degrees | 25-45 | degrees | 40 | degrees |
| piercing elements to the axis | | | | | | |
| of piercing | | | | | | |
| Blister diameter, C | 4-12 | mm | 6-9 | mm | 8.0 | mm |
| Blister depth, e | 2.0-3.50 | mm | 2.5-3.0 | mm | 2.8 | mm |

The preferred dimensions for the secondary piercing member 25 have been selected for evacuation from a circular blister 12 having a diameter of 8 mm and a depth of 2.8 mm. This size of blister 12 is sufficient to carry a dose of up to 5 mg of typical inhalable medicaments and provides a headspace in the blister 12 to facilitate straightforward loading of the drug into the blister 12 in high volume production. A preferred number of secondary piercing elements 26 on the secondary piercing member 25 is eight. In order to create an even airflow around the periphery of the blister 12 it is desirable to provide a large number of piercings therein. However, it is also necessary to open up a sufficient area of the foil lid 12b to allow free flow of the air through the blister 12. With many piercings in a given size of blister 12 either the holes have to become smaller or they have to be pierced so close to each other that the foil 12b between them is likely to tear during piercing. Eight secondary piercing elements 26 can easily be accommodated within the circumference of the blister 12 whilst still allowing each secondary piercing element 26 to open up a sufficient area of flow into the blister 12. A larger blister 12 may allow a secondary piercing member 25 with more piercing elements 26 to be used and a smaller blister 12 would allow fewer.

To facilitate even evacuation of the powder from the blister 12, the drug outlet tube 23 would ideally have a flat end (i.e. α=90 degrees). However, the tube 23 must also pierce a controlled cut into the lid 12b of the blister 12 and fully open a flap so that the powder exit is not impeded. If the angle α is close to 90 degrees a higher force is required to pierce the foil lid 12b and the drug feed tube 23 pierces the lid 12b in an uncontrolled manner. An angle of 60 degrees creates a controlled and repeatable cut in the foil 12b without unduly increasing the piercing force. The angle β influences how much pierced area is opened up to the airflow when the lid 12b is pierced. An angle close to 45 degrees is desirable to gain the greatest open area when fully pierced, as shown in FIG. 4. For a given length from the root to the tip of the primary piercing element 24, l, the greatest open area for flow is given when l cos β sin β is maximised. This occurs when β=45 degrees. A slightly lower value has been chosen (40 degrees) in the preferred embodiment, to make the piercing process more tolerant of variations in piercing depth due to tolerance variations from device to device.

The dimensions that have the most significant influence on performance are the depths of the secondary piercing member 25 and the outlet tube 23 in the pierced position. If the pierced area is too small, the airflow resistance of the blister increases and the evacuation of powder from the blister is reduced. The preferred ranges for the secondary piercing member 25 are chosen to open as much pierced area in the top of the blister as possible without the piercing elements 26 touching the blister base 12a or punching a contiguous ring through the lid 12b. The preferred ranges for the outlet tube 23 are chosen such that the tube 23 fully cuts and opens a flap in the lid 12b but does not go too close to the base 12a of the blister 12. In order to fully open a flap, the tube 23 must pierce a full diameter hole therein. (i.e. pierce to a depth below the lid 12b of >OD/tan α where OD is the outer diameter of the outlet tube 23 and α is as shown in FIG. 4). If the tube 23 is close to the base 12a of the blister 12, the flow of powder from the blister 12 up the tube 23 is impeded and evacuation of powder is reduced. The point of the primary piercing element 24 of the outlet tube 23 should be 0.2 mm and preferably greater than 0.5 mm from the base 12a of the blister 12.

An alternative embodiment of drug entrainment device which also promotes efficient evacuation from a foil blister 12 is illustrated in FIG. 5. In this configuration, the secondary piercing member 25 is replaced by a plurality of solid pointed piercing pins 30 arranged around the central drug feed tube 23. In use, the drug entrainment device 16 pierces the lid 12b and the blister 12 is then retracted by a small distance indicated by “C” in the Figure. Retraction of the blister 12 moves the pins 30 out of the apertures they have created to allow access to the interior of the blister 12 by the air flow passing down through the annular-conduit 21. In practice, the retraction mechanism would ideally comprise a cam arrangement associated with the blister 12 that causes the blister 12 to withdraw by a small distance once the lid 12b has been pierced. In this way, a number of peripheral inlet holes 31 are formed in the lid 12b of the blister 12 together with the central hole formed by the central piercing element 24.

Table 4 is a table comparing the performance of the second embodiment with that of the first embodiment. In these tests, the first embodiment provides improved evacuation from the blister, improved delivered dose and improved fine particle fraction of total dose. Furthermore, the first embodiment is preferred because no retraction mechanism is then required making the device simple to manufacture and operate. However, the performance of the drug entrainment device with retractable pins or retractable blister is also an improvement over known configurations.

TABLE 4
Blister evacuation and inhaler performance of the retracting drug
entrainment device and the non-retracting drug entrainment device

| | Delivered | FP dose as | % evacuation |
|---|---|---|---|
| Each result average of | dose as % of | % of total | from the |
| two MSLI tests | total dose | dose | blister |
| Retracting pierce head | 92.2% | 69.2% | 97.7% |
| Non-retracting piercing | 93.7% | 71.9% | 99.6% |
| star | | | |

In addition to altering the pattern and configuration of air inlets into and out of the blister 12, it has also been found that drug entrainment can be significantly improved by altering the shape of the secondary piercing member 25 to enhance the creation of a swirling airflow within the blister 12. Evacuation of the medicament from the blister 12 is thereby improved by ensuring that the internal surface thereof is completely swept by the gas flow.

Reference will now be made to the drawing of FIGS. 6A, 6B and 6C which illustrates a top plan view and two side elevational views of another embodiment of secondary piercing member 35 which would take the place of the secondary piercing member 25 mounted on the central feed tube 23 in the embodiment of FIG. 2. As can be seen, the secondary piercing member 35 now comprises a ring having a plurality of arms or blades 36 extending from a central aperture 37 in opposite directions (four being shown in the embodiment of FIG. 6) such that they extend substantially at right angles to the axis of the central feed tube 23 when the secondary piercing member 35 is mounted on the central feed tube 23 so that the central feed tube 23 extends through the aperture 37. On the side of the end of each arm 36 remote from the aperture 37, a flap is formed having an arcuately shaped outer periphery 38. Each flap is angled downwardly out of the plane of the arms 36 to form a vane surface 39 which is used to pierce the foil lid 12b. The vane surface 39 also serves to induce a swirling motion to the charge of compressed gas passing down through the annular conduit 21 and as it flows from the annular conduit 21 through the plenum chamber 22 and into the blister 12 via the openings therein created by the vanes 39 so as to cause the air to circulate around the blister 12 substantially around the axis of the central feed tube 23.

Although FIG. 6B shows the secondary piercing element 35 with the vanes almost entirely received within the blister 12, it will be appreciated that a proportion of the vane surfaces 39 may remain above and outside of the blister 12 so as to induce a swirling motion to the airflow within the plenum chamber 22 before it passes into the blister 12 through the apertures formed in the blister 12 by the vanes 39.

In a modified and preferred version of the aforementioned embodiment, as illustrated in FIGS. 7A and 7B, a swirling motion, indicated by arrow “B”, may be generated in the plenum chamber 22 above the blister 12 and secondary piercing member 35 by introducing some or all of the charge of compressed air into the plenum chamber 22 via a tangential gas inlet 40 rather than via the annular airflow conduit 21. In this case, the vanes 39 serve to maintain the swirling airflow generated in the plenum chamber as the air enters the blister. Without the vanes, a substantial portion of the swirling effect is lost as the air enters the blister and so the combination of the vanes and tangential flow inlet 40 prevent “straightening out” of the flow as it enters the blister 12.

The preferred dimensions and angles referred to on FIGS. 6C and 7B are shown in Table 5. The size of the star, or secondary piercing member, is related to the size of the blister. In a preferred embodiment, the blister diameter is 8 mm and its depth 2.8 mm. If a different sized blister were to be employed, the piercing star would be scaled accordingly. The vanes of the secondary piercing element have two functions: to open up a sufficiently large piercing to allow air flow and to promote, or at least not diminish, swirl in the air as it enters the blister. Accordingly, their size is chosen to be as large as can practicably be accommodated by the blister. The vane profile is chosen to match the curved profile of the blister bowl although they do not touch the sides of the blister when in the pierced position. The angle of the vanes is chosen to be close to 45° to the foil to open up the largest possible flow area for a given size of vane. In the preferred embodiment, four vanes are used. In an ideal case a large number of vanes would allow swirling flow to enter the blister uniformly at all points around the periphery of the blister. However, piercing at many points can cause the foil to tear in an uncontrolled and therefore undesirable manner. Four vanes provide a controlled pierce and allow sufficient airflow into the blister. A larger blister might allow more vanes and a small blister would accommodate fewer. The dimensions of the plenum chamber 22 are chosen to create a strongly swirling airflow above the blister that will be transmitted to the dose therein. The inlet is sized to present a minimal resistance to the airflow compared with the resistance of the vortex nozzle downstream of the blister. The remaining dimensions such as the internal diameter (d) of the drug feed tube 23, the depth of penetration (b) of the drug outlet tube 23 into the blister, the angle (α) of the face of the outlet tube 23 to its axis, the blister diameter (C) and, the blister depth (e) are all the same as those shown in Table 3.

TABLE 5
Preferred dimensions for the secondary piercing element and plenum
chamber of FIGS. 6 and 7

| | | | Most | | Most | |
|---|---|---|---|---|---|---|
| | Preferred | | preferred | | preferred | |
| Feature | range | | range | | value | |
| Span, B of the secondary | 4-9 | mm | 6-7.5 | mm | 7.2 | mm |
| piercing element | | | | | | |
| Height of the secondary | 1.2-2.0 | mm | 1.4-1.8 | mm | 1.6 | mm |
| piercing element, s | | | | | | |
| Width, w of piercing vanes | 1-3 | mm | | | 1.7 | mm |
| Number of secondary piercing | 2-8 | | | | 4 | |
| elements | | | | | | |
| Angle, β, of the piercing vanes | 30-60 | degrees | 35-55 | degrees | 45 | degrees |
| to the axis of piercing | | | | | | |
| Plenum diameter, D_{O} | 5-8 | mm | | | 6.8 | mm |
| Plenum inner diameter, D_{I} | 1.6-5 | mm | | | 3.8 | mm |
| Plenum height, H | 1-5 | mm | | | 3.75 | mm |

| | | | |
|---|---|---|---|
| Plenum inlet height, f | 30-100% of | 1.5 | mm |
| | plenum height | | |
| Plenum inlet projected width, g | 50-100% of | 1.5 | mm |
| | (D_{O} − D_{I})/2 | | |

As already mentioned, the introduction of a swirling airflow into the blister 12 increases the amount of medicament that is entrained in the airflow and evacuated from the blister 12 through the drug feed tube 23 to the aerosolising nozzle 2 and so the delivered dose and fine particle fraction of delivered dose is improved.

In addition to the foregoing, it is not always possible to ensure that the inhaler is used in the correct orientation by the user. It is therefore important that performance is not adversely affected for example when the inhaler is used upside down. A key benefit of introducing swirl to the powder in the blister is that the evacuation is less affected by the orientation of the inhaler.

Table 6, below, shows the results of tests with the inhaler device held upside down during piercing of the blister. Foil blisters were filled with 3 mg of sodium cromoglycate and then tested in a device with a reservoir volume of 15 ml and a reservoir gauge pressure of 1.5 bar. The emitted dose was measured using a DUSA apparatus and wet chemical assay to evaluate the quantity of drug. Five consecutive shots were evaluated in this way and the mean and RSD (=standard deviation/mean) calculated.

With the standard plenum and secondary piercing element of FIGS. 2 and 4, the emitted dose drops by 9 percentage points when the blister is pierced upside down. The dose to dose variation over five shots is also significantly worse when pierced upside down with the RSD increasing from 2% to 10%. With the tangential airflow inlet to the plenum chamber 22 and the secondary piercing element of FIGS. 6 and 7, the mean emitted dose is improved and the change in performance when piercing the blister upside down is reduced to 3 percentage points. Importantly, the dose to dose variation over five shots is the same whether the blister is pierced upside down or in the correct orientation. This is a significant benefit over the standard arrangement because the swirl arrangement will be able to achieve more consistent dosing regardless of the orientation of use.

TABLE 6
Effect of inhaler orientation with standard piercing arrangement
and swirl generating piercing arrangement

| | | Swirling flow in the blister | |
|---|---|---|---|
| Standard piercing arrangement | | (secondary piercing element of | |
| (secondary piercing element and | | FIG. 6 and tangential inlet to | |
| inlet to plenum as in FIG. 2) | | plenum of FIG. 7) | |
| Correct pierce | Pierced upside | Correct pierce | Pierced upside |
| orientation | down | orientation | down |
| Mean ED: 86% | Mean ED: 77% | Mean ED: 96% | Mean ED: 93% |
| RSD: 2% | RSD: 10% | RSD: 2% | RSD: 2% |

Table 7 shows the results obtained when the embodiment of FIG. 2 is tested with the secondary piercing member used in FIG. 11 and, when the embodiment of FIG. 11 is used with the secondary piercing element of FIG. 3. This shows that the best performance is obtained when the tangential airflow inlet to the plenum chamber 22 is combined with the secondary piercing element of FIG. 11.

TABLE 7
Effect of inhaler orientation with combinations of standard and swirl
piercing arrangements

| Standard plenum (as in FIG. 2) | | Tangential inlet to plenum (as in | |
|---|---|---|---|
| with secondary piercing | | FIG. 7) with secondary piercing | |
| element of FIG. 6 | | element of FIG. 2 | |
| Correct pierce | Pierced upside | Correct pierce | Pierced upside |
| orientation | down | orientation | down |
| Mean ED: 78% | Mean ED: 83% | Mean ED: 89% | Mean ED: 87% |
| RSD: 12% | RSD: 8% | RSD: 7% | RSD: 10% |

It has also been found that with a vortex nozzle aerosolising system it is desirable that the maximum loading of powder going through the nozzle (i.e. mass of powder per second) is kept below a threshold. Above this threshold the nozzle can become overloaded and its efficiency is reduced and this has a detrimental effect on the delivered dose. It is therefore desirable to spread out the introduction of the powder to the nozzle over a period of time so that the powder density in the nozzle is kept sufficiently low to maintain the nozzle's efficiency.

A further benefit of generating swirl in the blister is that the time over which the powder is entrained in the airflow is increased, thus helping to achieve a more even flow of powder into the aerosolising nozzle.

Dose Storage Pack

In addition to providing devices which enhances the evacuation of the drug from a conventional blister 12, the inventors have also developed a new type of medicament pack for storage of a drug dose especially for use with a dry powder inhaler which is designed to minimise restriction to the gas flow from the pressurised gas source to the aerosolising nozzle as well as generate a swirling air flow between the air inlet and outlet to the packaging so as to entrain the drug and evacuate substantially all of the drug from the pack.

Two preferred embodiments of medicament pack according to the invention are illustrated in FIGS. 8A and 8B. FIG. 10 is a table showing the percentage of drug (3 mg sodium cromoglycate—the entrainment device was attached to airflow control apparatus set up to deliver a flow rate of 21 pm for a period of 3 seconds, apart from the embodiment of FIG. 8B which was tested at 31 pm) evacuated using each of these chamber designs together with the results obtained using a number of other packages illustrated in the cross sectional views of FIGS. 8C to 8G, as well as a conventional gelatin capsule, for comparison purposes.

As can be seen, the inventors have found that very efficient entrainment of dry powder is obtained when the dose is contained in a cylindrical swirl chamber 45 having facing opposite end walls and a tangential inlet 46 and outlet 47, the inlet 46 and outlet 47 being situated at opposite ends of the swirl chamber 45, as shown in the embodiment of FIG. 8A and 13AA showing a perspective view, and two-cross-sectional views, respectively. Preferably, the chamber diameter is 4 mm and its length is 7 mm.

Slightly less efficient entrainment is obtained when the dose is contained in a cylindrical swirl chamber 48 provided with a tangential inlet 49 and an outlet 50 coaxial with the longitudinal axis of the chamber, as shown in the perspective view of FIG. 8B.

When one of the aforementioned medicament packs are used, the outlet of the swirl chamber 47, 50 is connected to an aerosolising nozzle and the swirl chamber inlet 46, 49 is connected to a valve which is in turn connected to a source of pressurised gas. In use, when the valve is opened, for example, in response to the user's inhalation, a charge of pressurised gas flows into the chamber 45, 48 creating a swirling flow from the inlet 46, 49 to the outlet 47, 50, due to the shape of the chamber 45, 48, which scours a very high proportion of the dry powder dose and delivers it through the outlet 47, 50 to the aerosolising nozzle.

Another embodiment of medicament pack according to the invention is illustrated in the cross-sectional view of FIG. 9. As can be seen, the pack 51 comprises a plastic moulded housing 52 in the form of a short tube with open ends. A piercable foil laminate 53a, 53b seals each open end. When the pack 51 is to be used, the foil 53a is pierced to allow an airflow inlet tube 54 to penetrate into the pack 51 and the foils 53b is pierced to allow a drug outlet tube 55 which communicates with an aerosolising nozzle 55a to penetrate into the pack. The foils 53a, 53b are pierced such that the air must pass substantially through the whole of the pack before it reaches the outlet so that the dose contained therein is entrained in the airflow. This type of pack may be used with a number of inhalers each having a different design as the pack can be pierced on both sides or just on one side, as with a conventional blister pack.

As previously mentioned, any deposition of drug within the device can have a significant effect on the variation of the delivered dose in successive uses of the device as well as on the fine particle fraction of total dose. Therefore, it is desirable to minimise the components of the device with which the drug entrained in the airflow can come into contact. To this end, the present invention also provides a medicament pack in which the drug storage chamber, the aerosolising nozzle and the drug feed tube between the nozzle and the blister are formed together in a single use integrated module that is discarded after each time the device is used. FIGS. 11A to 11G illustrate various embodiments of drug packages incorporating one or more aerosolising nozzles according to the invention. A preferred embodiment of pack 60 is illustrated in FIG. 11A in which the aerosolising nozzle 61 and the dose storage blister 62 are both formed from a cold formed foil base 64 covered with a puncturable lidding foil 65. The lid 65 is sealed to the base 64 preferably by heat sealing. The dose storage chamber 62 may be shaped as a half cylinder so as to promote the swirling flow of air as it enters via an inlet 66 formed therein as a result of piercing the lidding foil 65. The other chamber 61 may be configured as a nozzle or vortex chamber with a tangential inlet 67 and a central axial outlet 68 which is also formed by piercing the lid 65. When a charge of pressurised gas is passed into the drug storage chamber 62 via the inlet 66, the dose contained in the chamber 62 is entrained in the airflow. The entrained dose flows into the nozzle 61 via an intermediate conduit 69 between the drug storage chamber 62 and the nozzle 61 where the dose is aerosolised by the action of shear forces, turbulence and impaction. The aerosolised dose leaves the nozzle 61 via the outlet port 68. Preferably, the diameter of the nozzle 61 is 8 mm and its depth is in the range 1.0 to 2.8 mm.

A modified version of the preferred embodiment of FIG. 11A is illustrated in FIG. 11B. In this arrangement, the dose storage chamber 66 is cylindrical in shape has a tangential inlet 70 from an additional inlet cavity in which the inlet 66 is pierced by the inhaler.

Another embodiment is illustrated in FIG. 11C. Instead of forming the dose storage chamber 62 and aerosolising nozzle 61 from foil using cold forming, the dose storage chamber 62 and nozzle 61 are formed from a plastic moulding 72 onto which the lid 65 is sealed, as with the embodiments of FIGS. 11A and 11B. The advantage of moulding the nozzle 61 and dose storage chamber 62 allows greater accuracy and definition to be achieved in the geometry of the chambers 61,62 than is achievable when the dose storage chamber 62 and nozzle 61 is formed entirely of foil.

FIG. 11D shows a modified version of the combined dose storage chamber 62 and nozzle 61 of FIG. 11C. Instead of forming the outlet 68 from the nozzle 61 in the lidding foil 65, an outlet 73 is formed in the moulded plastic component which may be sealed with a foil flap 74 prior to use and which is pealed away to open the outlet 73. This improves the definition achievable in the geometry of the outlet 73.

Another embodiment is illustrated in FIG. 11E. In this version, there is no intermediary conduit 69 between the drug storage chamber 62 and the nozzle 61. Instead, this is formed in the inhaler which pierces an outlet 75 for the drug in the foil covering the drug storage chamber 62 in addition to the inlet 66. The inhaler must also pierce an opening in the lid 65 covering the nozzle 61 to form an inlet for the compressed air together with the drug entrained therein. The outlet 73 may be formed in the plastic moulding as described with reference to FIG. 11D. The advantage of this arrangement is that the powder is contained in the dose storage chamber 62 and cannot migrate into the vortex chamber 61 until the lid 65 is pierced when the pack is used.

Further arrangements are shown in FIGS. 11F and 11G. In the embodiment of FIG. 11F, multiple drug storage chambers 62 are shown which feed a single aerosolising nozzle 61. It will be appreciated that this embodiment is not as efficient as those which embody a single use nozzle as deposition of drug may occur during, for example, evacuation of the first dose storage chamber 62a, which will have an affect on the delivered dose when the second and/or third dose storage chambers 62b, 62c are used together with the same nozzle 61. FIG. 11G illustrates multiple dose storage 62a, 62b, 62c and nozzle 61a, 61b, 61c pairs in a single assembly. Preferably, the dose storage and vortex chambers are formed from cold formed foil covered with piercable lidding foil.

Although the embodiments described in this part of the application refer primarily to active, i.e. powered, dry powder dispersion inhalers, the concepts apply equally to passive dry powder inhalers where the dispersion energy is provided by the user of the device. As will be appreciated by a skilled person, the dimensions of the air pathways through the entrainment blister or chamber and the aerosolising nozzle would need to be enlarged in order to provide a sufficiently low pressure drop for passive inhalation. For example, this could be achieved by scaling up the size of the device in proportion.

Valve Enhancements

As discussed briefly above, it is necessary to ensure that the dry powder including the therapeutically active agent is completely expelled from the pack in which it is stored as well as from the delivery device so that there is minimal deposition within the device. Another way of achieving this will now be described below.

To increase the efficiency of entrainment of the dose, it is important that the valve which releases the charge of compressed gas is opened quickly so that the charge enters the blister over a very short period of time and the dose receives sufficient fluid energy from the gas so that all or substantially all of the dose is entrained in the airflow. If the valve opens slowly, the dose will receive the charge of gas over a longer period with less energy and so some of the dose may not be entrained in the airflow resulting in a reduction in the efficiency of the device.

It will be appreciated from the foregoing, that a valve is required that both opens rapidly and, presents a minimum resistance to flow once open. The speed by which a valve opens may be defined by the shortest time between the valve being fully closed and the valve being fully open. Additionally, it is also desirable that the forces required to operate the valve are as low as possible to reduce strain on components and facilitate ease of operation.

The effort required to keep a valve closed against a pressure is called the sealing force. The sealing force comprises two components: the pressure force Fₚ and the seat force Fₛ. The pressure force is the force generated by the pressure within a chamber and is given by the equation Fₚ=PA, where P is the pressure acting on the valve and A the area over which the pressure acts. Depending on the configuration of the valve, the pressure force may act to bias the valve towards the open or the closed position. The seat force, Fₛ, is the force required to create a continuous loop of intimate contact between the compliant part of the valve (the seal) and the valve seat.

An inhaler having a valve which is sealed by an immobilising mechanism and arranged so that the pressure acting on the valve acts to bias it towards an open position is known from U.S. Pat. No. 6,029,662. Although the valve opens rapidly because the compressed gas biases the valve to the open position and so assists opening, it is possible for the valve to leak because the closing mechanism has to oppose the pressure force generated in the chamber rather than use this pressure force to assist sealing. Therefore, in practice a high closing force to ensure sealing is required. A further disadvantage with this type of valve is that it must be re-set prior to re-pressurisation of the chamber.

To reduce the pressure force that must be overcome to seal the valve, the area of the valve exit orifice is minimised. However, this introduces the additional drawback that the speed of flow through the valve is considerably reduced so that although the valve opens rapidly, the speed at which the chamber empties is limited by the small size of the valve exit orifice.

In an alternative valve configuration, the pressure in the chamber biases the valve into a closed position to reduce the risk of leakage. The advantage of this approach is that the only force required to keep the valve closed is the seat force and this force may be provided by the pressure force. However, to open the valve, the pressure force acting on it must be overcome and this requires an actuation force much greater than the pressure force, especially if the valve is to be opened rapidly.

It will be appreciated from the foregoing that each of the above described types of valve embody an undesirable compromise. With a valve configuration of the first type, the valve opens rapidly but requires high forces to hold the valve closed and needs to be reset, for example by manually resetting. In the second case, the valve has a low closing force and can potentially be self-resetting, but a high opening force is needed for rapid opening.

The present invention seeks to provide a dry powder inhaler having a valve that overcomes or substantially alleviates the disadvantages associated with an inhaler having either of the types of valve described above.

According to one embodiment of the invention, there is provided a dry powder inhaler for delivering a dose of medicament for inhalation by a user, including a drug entrainment device and a valve actuable by a user to cause pressurised gas to flow through a dose of medicament disposed in the drug entrainment device to entrain said dose in the gas, the valve comprising a valve member configured such that, in a first mode, pressurised gas biases the valve member into an open state to allow the flow of gas through the valve and, in a second mode, pressurised gas biases the valve member into a closed state to prevent the flow of gas through the valve. Although reference is made to pressurised gas, it should be understood that this includes compressed air in addition to gases.

Preferably, the valve is configured such that pressurised gas acts over both sides of the valve member when it is in the closed state. Although the pressure of the gas acting over each side of the valve member may be the same, it may act over a larger cross-sectional area of one side of the valve member than the pressurised gas acting over the other side of the valve member. This means that for the same given pressure, the force acting over a greater cross sectional area of the valve will be larger. As the force generated over one side of the valve member is larger, the valve member is maintained in a closed state.

In a preferred embodiment, the valve is configured such that the valve member moves from the closed state to the open state in response to a change in pressure of the gas acting on one side of the valve member relative to the pressure acting on the other side of the valve member.

The inhaler preferably comprises a reservoir for pressurised gas and a valve orifice for the passage of pressurised gas from the reservoir through the drug entrainment device. A first side of the valve member forms a seal with the valve orifice when in the closed state such that pressurised gas in said reservoir acts over only a portion of said first side of the valve member defined by the cross-sectional area of the valve orifice.

Conveniently, the valve orifice is located at the mouth of a tube in communication with the reservoir, the tube including a valve seat at the end thereof for cooperation with said first side of the valve member to form a seal therewith when the valve member is in the closed state.

The valve is preferably configured such that when the seal between the first side of the valve member and the valve seat is broken, the pressure of the gas in the reservoir acts over substantially the entire surface of the first side of the valve member to bias the valve member into the open state. As the pressure acting over one side of the valve is discharged, a threshold is reached at which the pressure of the gas in the reservoir acting over the other side of the valve is sufficient to cause the valve member to lift from the valve seat. When this occurs, the whole of the underside of the valve member is exposed to the pressure of the gas in the reservoir causing it to open rapidly.

In one embodiment the inhaler includes biasing means to bias the valve member into a closed state when the pressure of the gas in the reservoir has been discharged through the valve. This re-sets the valve member automatically into the closed state and removes any need to pressurise the other side of the valve member in advance of pressurisation of the reservoir.

The biasing means may conveniently comprise a spring.

In a preferred embodiment, means are provided to discharge the pressure that biases the valve member into the closed state to cause the valve member to move from the closed to the open state.

The valve preferably includes a primary chamber in which pressure to bias the valve member into the closed state is generated and said means for discharging the pressure that biases the valve member into the closed state comprises a discharge port in the primary chamber.

The valve advantageously includes means for opening the discharge port to atmosphere. Most advantageously, the means for opening the discharge port is breath actuated.

When the valve is breath actuated, it preferably includes a secondary valve member which is movable, in response to inhalation by a user, from a first closed position in which the discharge port is not in communication with the primary chamber to prevent discharge of the primary chamber to the atmosphere, into a second open position in which the discharge port is in communication with the primary chamber to discharge the primary chamber to the atmosphere.

The secondary valve member is preferably configured such that the pressure in the primary chamber acts over a smaller cross-sectional area of a first side of the secondary valve member than the cross-sectional area of the other side of the valve member over which atmospheric pressure acts, when the secondary valve member is in the closed position.

Conveniently, the valve member and secondary valve member may be flexible diaphragms.

The inhaler also preferably includes means for charging the reservoir with pressurised gas or air. Most preferably said means is also operable to charge the primary chamber.

A conduit may communicate the reservoir with the primary chamber to facilitate the charging of the primary chamber during charging of the reservoir with pressurised gas.

Embodiments of the invention will now be described, by way of example only, and with reference to FIGS. 13 to 20 of the accompanying drawings, in which:

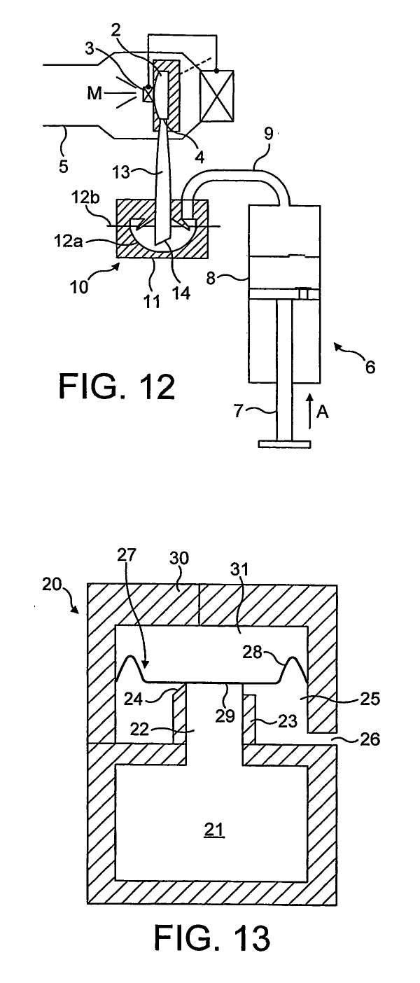

FIG. 12 is a schematic drawing of a conventional pressurised gas powered active dry powder inhaler;

FIG. 13 is a simplified cross-sectional side elevation of a valve assembly according to the invention;

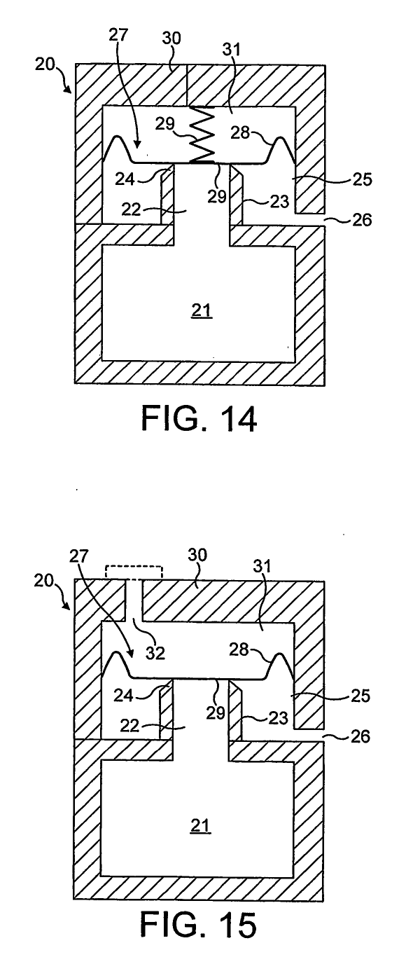

FIG. 14 is a first modified version of the valve assembly illustrated in FIG. 13;

FIG. 15 is second modified version of the valve assembly illustrated in FIG. 13;

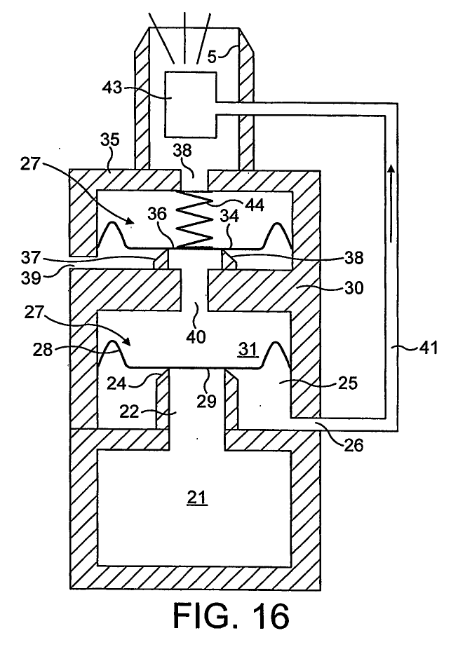

FIG. 16 is a third modified version of the valve assembly illustrated in FIG. 13;

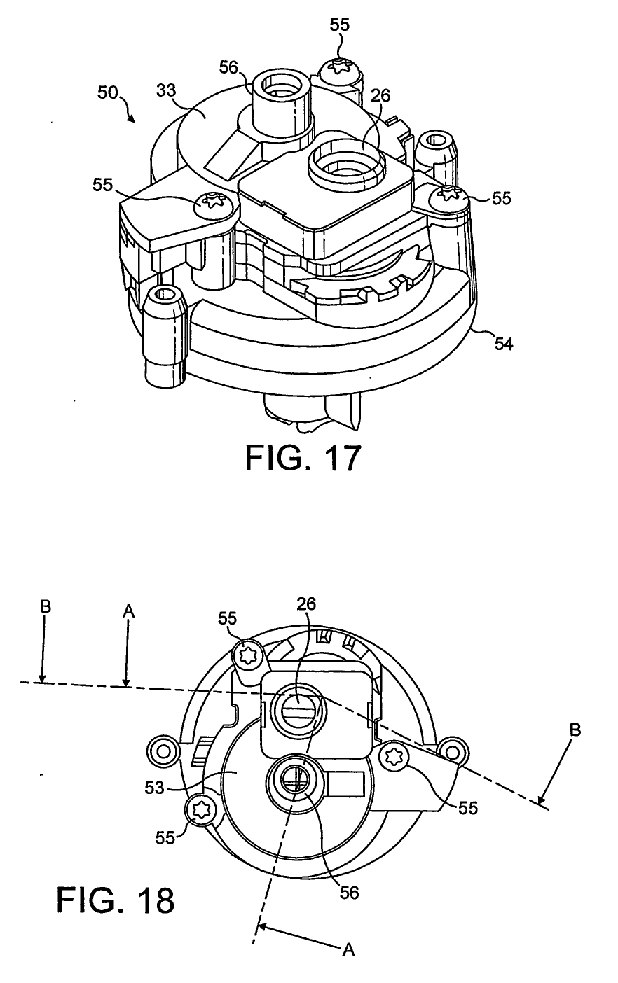

FIG. 17 is a perspective view of an actual breath actuated valve module forming part of an inhaler according to the invention;

FIG. 18 is top plan view of the breath actuated valve module shown in FIG. 17;

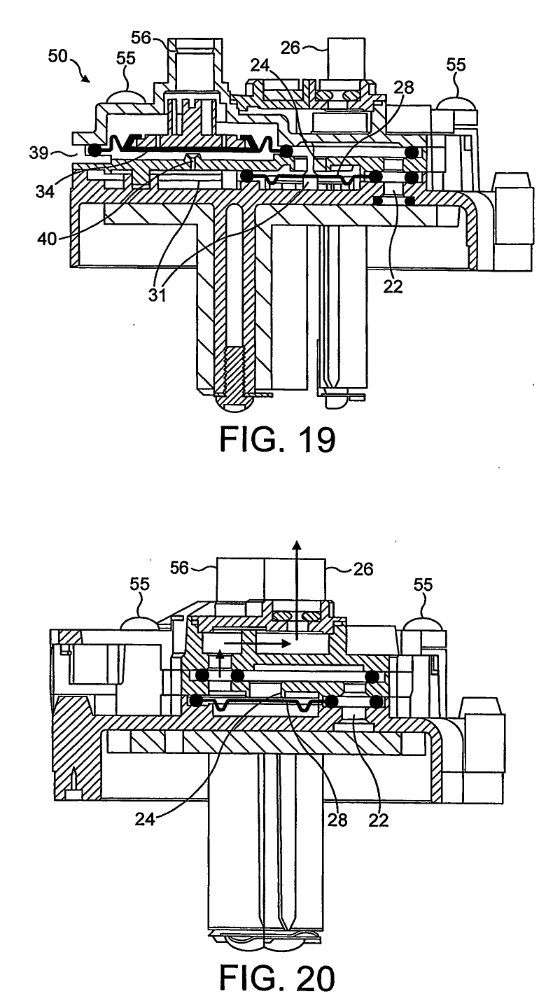

FIG. 19 is a cross-sectional side elevation of the breath actuated valve module taken along the section A-A in FIG. 18; and

FIG. 20 is a cross-sectional side elevation of the breath actuated valve module taken along the section B-B in FIG. 18.

A schematic drawing of a conventional gas powered dry powder inhaler for aerosolising a powdered medicament for inhalation by a user is illustrated in FIG. 12. The inhaler 1 comprises a vortex chamber or nozzle 2 having an exit port 3 and an inlet port 4 for generating an aerosol of medicament M. The nozzle 2 is located within a mouthpiece 5 through which a user inhales the aerosolised medicament M.

The dose is supplied to the nozzle 2 in an airflow generated by a pump represented in FIG. 12 as a piston pump 6 containing a plunger 7 received in a pump cylinder 8. An airflow path 9 extends from the pump cylinder 8 to a drug entrainment device 10 comprising a housing 11 to support a foil blister 12 containing a single dose of medicament (typically between 0.5 and 5 mg). The blister 12 has a cold-formed foil blister base 12a sealed with a hard rolled foil laminate lid 12b chosen to facilitate piercing. A drug feed tube 13 extends from the inlet port 4 of the nozzle 2 and into the housing 11 where it terminates in a piercing element 14. When the inhaler 1 is to be used, the pump 6 is primed with a charge of compressed air by sliding the plunger 7 into the pump cylinder 8 (in the direction of arrow “A” in FIG. 12) to compress the air contained therein. Thereafter, the housing 11 and the drug feed tube 13 and moved relative to each other to cause the piercing element 14 to break the foil laminate layer 12a and penetrate into the blister 12 so that when the user inhales through the mouthpiece 5 a valve 15, which may be breath actuated, releases the charge of compressed gas from the cylinder 8 so that it flows down the airflow path 9 into the blister 12 and up through the drug feed tube 13. As the air passes through the blister, the dose contained therein is entrained and is carried by the airflow up the drug feed tube 13 and through the inlet port 4 into the nozzle 2.

A rotating vortex of medicament and air is created in the nozzle 2 between the inlet and outlet ports 4, 3. As the medicament passes through the nozzle 2, it is aerosolised by the high turbulent shear forces present in the boundary layer adjacent thereto as well as by the high level of turbulence in the vortex chamber and through collisions between agglomerates and other agglomerates and between agglomerates and the walls of the nozzle 2. The aerosolised dose of medicament and air exit the nozzle 2 via the exit port 3 and is inhaled by the user through the mouthpiece 5

FIGS. 13 to 16 represent three highly simplified representations of valves that operate according to the principle of the invention and reference is first made to them for the purpose of explanation and to facilitate understanding of the invention.

Referring now to FIG. 13, there is shown an assembly 20 comprising a reservoir 21 containing a source of compressed gas or air. The reservoir 20 may be charged using a variety of means including a piston pump, a multiple action pump charging an accumulator via a check valve, a canister of compressed gas or a canister of propellant such as HFA. The reservoir 21 has a compressed gas outlet orifice 22 defined by a tube 23 terminating in a seat 24 through which gas may pass from the reservoir 20 via a servo chamber 25 and out of the assembly 20 through an exit orifice 46 to drug aerosolising means via a drug entrainment device (not shown). A valve member 27 is associated with the outlet orifice 21 to selectively permit or prevent the flow of compressed gas from the reservoir 21 into the servo chamber 25.

The valve member 27 comprises a flexible diaphragm 28 which extends across the end of the tube 22. A central region 29 of the diaphragm contacts the seat 24 to make a seal therewith when the valve is closed. It will be appreciated that only a relatively small central region 29 of the underside of the diaphragm 28 will be exposed to the effects of the pressure acting against it due to the source of compressed gas in the reservoir 20. The size of this region depends on the internal cross-sectional area of the tube 23.

The diaphragm 28 is located within and extends between the walls of a housing 30 to define a space or primary chamber 31 above the diaphragm 28, for reasons that will now be described.

It will be appreciated that when the reservoir 21 is pressurised to a pressure Pᵣₑₛ, a pressure force will be acting over the central region 29 of the diaphragm 28 which will tend to cause the diaphragm 28 to lift off the seat 24 and thus allow the gas to escape from the reservoir 21. To counteract this pressure force against the central region 29 of the diaphragm 28, the primary chamber 31 is also pressurised to a pressure Pₚ such that the force acting against the opposite side of the diaphragm 28 is sufficient to hold the central region 29 against the seat 24 and therefore keep the valve closed. The sealing force that must be generated by the pressure Pₚ in the primary chamber 31 which is sufficient to keep the valve closed is the sum of the seat force Fₛ of the diaphragm 28 against the seat 24 and the force Fₚ due to the pressure Pᵣₑₛ acting on the diaphragm 28 over the central region 29 of the diaphragm 28. Typically, the primary chamber 31 only needs to be pressurised to the same pressure as the reservoir 21, i.e. Pₚ=Pᵣₑₛ to keep the valve closed. This is because the pressure Pₚ acts over a much greater surface area of the diaphragm 28 than does the pressure Pᵣₑₛ.

The diameter of the tube 23 may be sufficiently large so as not to impede flow once the diaphragm 28 is open. The cross-sectional area of the tube 23 is limited only by needing to be smaller than the total cross sectional area of the diaphragm 28 so that the net force acting on the diaphragm is sufficient to ensure that its central region 29 seals against the valve seat 24, i.e. net force>seat force Fₛ.

To open the valve, it is necessary to lift the diaphragm 28 so that the seal is broken between the central region 29 of the diaphragm 26 and the seat 24. To do this, the diaphragm 28 can be lifted using a mechanical device (not shown). It will be appreciated that once the diaphragm 28 has been unseated, the pressure Pᵣₑₛ will now act over the whole of the underside of the diaphragm 28 rather than just the central region 29 thereof. As a result, the sealing force required to keep the valve closed and the force due to the pressure in the chamber 31 acting over the upper side of the diaphragm 28 will be equalised. As the net force now acting on the diaphragm 28 is zero, the valve opens rapidly.

To reset the valve by moving the diaphragm 28 back to its original closed position in which it locates against the seat 24, the primary chamber 31 is pressurised before the reservoir 20 so that the net force on the diaphragm 28 exceeds the required seat force between the central region 29 of the diaphragm 28 and the seat 24.

A first modified version of the assembly described with reference to FIG. 13 is shown in FIG. 14. In this arrangement, advanced pressurisation of the primary chamber 31 is rendered unnecessary as a biasing means, such as a spring 29, is disposed between the diaphragm 28 and the housing 30 and serves to bias the central region 29 of the diaphragm 28 against the seat 24 thereby making the valve self-resetting.

A second modified version of the assembly described with reference to FIG. 13 is shown in FIG. 15. In this arrangement, the diaphragm 26 is lifted from its seat 23 to open the valve by allowing pressure in the chamber 31 to decay to a point at which the force Fₚ due to the pressure acting on the diaphragm 28 is not longer sufficient to hold the central region 29 of the diaphragm 28 against the seat 24. Preferably, the pressure is allowed to decay by opening a port 32 in the housing 30 to communicate the chamber 31 to the atmosphere. This embodiment is particularly advantageous because the reservoir pressure Pᵣₑₛ acts to force the diaphragm 28 open therefore the discharge from the reservoir 21 is particularly rapid.

Although a mechanical device can be provided for opening and closing the port 32, the modified version of FIG. 13 can be adapted so that the port opens in response to the user's inhalation, as will now be described with reference to FIG. 16. For this purpose, the assembly is provided with a secondary valve member 33 which may be a breath actuated diaphragm 34, a vane or piston (not shown) mounted in a second housing 35 in a similar manner to the first diaphragm 28. The breath actuated diaphragm 34 has a central region 36 which seals against a seat 37 formed at the end of a tube 38 which extends from an aperture 40 that communicates the primary chamber 31 with the underside of the central region 36 of the breath actuated diaphragm 34 to block the flow of air from the primary chamber 31 to a primary chamber dump port 39 which is open to atmosphere. The upper surface of the secondary diaphragm 34 is in communication with the mouthpiece 5 via an opening 38.

When a user inhales through the mouthpiece 5, the central region 36 of the breath actuated diaphragm 34 is lifted from its seat 37 due to the lower pressure created in the mouthpiece 5 which is transmitted to the upper surface of the breath actuated diaphragm 34 via the opening 38. When the breath actuated diaphragm 34 is unseated, the primary chamber 31 is opened to the atmosphere via the aperture 40, the tube 38 and the primary chamber dump port 39. When this occurs, the pressure in the primary chamber 31 reaches a threshold at which the diaphragm 28 lifts rapidly releasing the charge of compressed gas from the reservoir 21 through the servo chamber 25 and the exit orifice 26 to deliver the dose of medicament via an airflow conduit 41 to a drug entrainment device and aerosolising means 43. It will be appreciated that when the breath actuated diaphragm 34 is lifted from its seat 37 when the user inhales, the pressure of the gas in the primary chamber will then act over the whole of the cross-sectional area of the underside of the breath actuated diaphragm rather than just over the central region 36. The pressure of the air in the primary chamber 31 therefore assists the breath actuated diaphragm 34 to open.

A biasing means such as a spring 44 acts against the breath actuated diaphragm 34 so that when the charge of gas in the primary chamber 31 has discharged, the breath actuation diaphragm 34 is automatically returned to the closed position by the spring 44. This arrangement allows the breath actuation diaphragm 34 to be self-resetting without the need for a separate resetting action by the user.

It will be appreciated that the valve uses a servo type action. When the diaphragm 28 is opened to a certain extent, high pressure air from the reservoir 21 floods the servo chamber 25 below the diaphragm 28 which then empties via the downstream drug entrainment and aerosolising means 43. If the flow resistance of the downstream entrainment device and aerosolising means 43 is much greater than that of the tube 22, the pressure in the servo chamber 25 will rapidly become almost equal to the reservoir pressure 21. This pressure acts on the underside of the diaphragm 28 and holds it open whilst the reservoir 21 is discharged.

It has been found by the inventors that the diameter of the chamber dump port 39 needs to be sufficiently large to facilitate rapid discharge of the primary chamber 31. If the primary chamber 31 is too small, the breath actuated diaphragm 34 can “bounce” or “flutter” causing the primary chamber 31 to discharge in stages compromising the efficiency of the inhaler. The cross-sectional area of the chamber dump port 39 should be greater than 0.15 mm² and should preferably be between 0.15 mm² and 0.75 mm². In a most preferable embodiment, the cross-sectional area of the chamber dump port 37 is 0.4 mm². If the dump port 39 has a cross-sectional area less than 0.15 mm², a delay is introduced between movement of the second diaphragm and the opening of the main valve diaphragm 26. Such a delay is undesirable, although if the dose is to be delivered later during an inhalation by the user, the dump port 39 could be designed so as to introduce a desired delay.

Although the chamber 31 can be provided with its own means to enable it to be pressurised, it is particularly desirable to use the means for charging the reservoir 21 to also charge the chamber 31. This can be achieved by, for example, incorporating a port (not shown) communicating the chamber 31 with the reservoir 21 which is closed prior to actuation of the valve.

The presence of a port between the reservoir 21 and the chamber 31 also prevents premature firing of the valve in the event of a leak from between the breath actuated diaphragm 34 and its seat 37 which can be caused due to, for example, imperfect sealing as a result of dirt ingress therebetween. As the diaphragm 28 is designed to open when the pressure difference between the primary chamber 31 and the reservoir 21 drops below a particular threshold, the possibility exists that a leak could cause the valve to open prematurely wasting the drug dose. However, it has been found that the diaphragm 28 will not servo open if the pressure is reduced sufficiently slowly and will instead open fractionally to allow gas to escape so that the reservoir pressure will drop in proportion to the slowly decreasing pressure in the chamber 31.

The assembly may be additionally provided with a control orifice (not shown) communicating the primary chamber 31 with the reservoir 21 so that any pressure drop in the chamber 31 due to a leak therein which is smaller than the control orifice constriction will be topped up from the reservoir 21.

Reference will now be made to the breath acutated valve module 50 forming part of an actual dry powder inhaler according to the invention which is illustrated in FIGS. 17 to 20. The breath actuated valve module 50 works as described with reference to FIGS. 13 to 16 and so like components will be referred to by the same reference numerals for ease of understanding.

A perspective view of the breath actuated valve module is shown in FIG. 17 and comprises an upper casing part 53 mounted on a lower casing part 54 using screws 55. The exit 26 through which the compressed air flows from the module to the aerosolising nozzle via the drug entrainment device can be seen, as can a connector 56 which connects the valve module 50 the mouthpiece and through which the breath actuated diaphragm is controlled in response to inhalation by a user.

FIG. 18 illustrates a top plan view of the module 50 shown in FIG. 17 and FIGS. 19 and 20 illustrate two cross-sections taken along the lines A-A and B-B respectively. The cross-sectional illustrations show the outlet orifice 22 from the reservoir 21 and the tube 22 with the diaphragm 28 seated against the valve seat 28. The primary chamber 31 extends across the module and discharge of the compressed air from this chamber 31 through the chamber dump port 39 is selectively prevented by the breath actuated diaphragm 34 which is located against the valve seat 37 at the end of tube 38.

Powder Entrainment & De-Agglomeration

Upon actuation of the dispensing device, the powder formulation becomes entrained in an airflow which is generated (actively or passively) within the device. The manner in which the powder becomes entrained in this airflow and is then expelled from the device is also crucial in ensuring that as much of the active agent is dispensed as possible.

It is not simply a question of entraining as much of the powder as possible in the airflow. In addition, the entrainment should be such that the plume of powder expelled from the device is such that deposition of the active agent in the throat is minimised. Finally, it is also desirable for any agglomerates in the powder to be broken up as the powder becomes entrained in the airflow.

This deagglomeration is possible where the airflow is controlled so that it applies shear forces on the powder formulation as it becomes entrained in the airflow. These shear forces can serve to break up agglomerated particles, thereby enhancing the FPF and FPD of the powder.

One way in which deagglomeration of agglomerates in the dry powder formulation may be achieved during powder entrainment within the dispensing device it to arrange the airflow so that it applies shear forces to the powder, breaking the agglomerates apart.

Whilst this may occur, as discussed above, in connection with the emptying of the blister or capsule in which the individual doses are held prior to actuation of the inhaler device, such deagglomeration may also occur as the powder becomes entrained in the airflow.

In addition to deagglomeration, it is also very important for the entrainment of the powder in the airflow to be as efficient as possible, leaving at little powder behind. Finally, another consideration is the dynamics of the powder as it leaves the inhaler device. Once again, this is linked to the entrainment of the powder in the airflow. As discussed below in greater detail, the movement of the active particles in the plume created by the inhaler will affect the amount of active agent which is deposited in the throat of the user, rather than in the lung.

Naturally, the entrainment of the dry powder formulation in an airflow will be affected by the properties of the formulation itself, as well as the device used. For example, entrainment of a fine powder, that is, one which does not include a population of larger particles, such as carrier particles is more difficult than entrainment of a powder comprising a combination of large and fine particles. However, the arrangement of the device itself also affects the powder entrainment. In particular, it is the path of the airflow through the powder and out of the device which will determine any deagglomeration, powder entrainment and powder velocity, etc.

According to an aspect of the present invention, a method is provided comprising entraining agglomerated particles in a gas flow. The method comprises depositing the agglomerate particles onto one or more surfaces, and applying, via the gas flow, a shear to the deposited agglomerated particles to deagglomerate them.

In one embodiment, the method comprises entraining a powdered substance in a gas flow stream from an inlet port of a vortex chamber having a substantially circular cross-section. The method further comprises directing the gas flow through the vortex chamber in a tangential direction; directing the gas flow through the vortex chamber so as to aerosolise the powder composition; and directing the gas flow with the powder composition out of the vortex chamber in an axial direction through an exit port. Preferably, the velocity of the gas flow at a distance of 300 mm outside of the exit port is less than the velocity of the gas flow at the inlet port.

In another embodiment, the method comprises entraining a powdered composition including agglomerated particles in a gas flow upstream from an inlet port of the vortex chamber. In this embodiment, the method comprises directing the gas flow through the inlet port into the vortex chamber; depositing the agglomerated particles onto one or more of the walls of the vortex chamber; applying, via the gas flow through the vortex chamber, a shear to the deposited agglomerated particles to deagglomerate the particles; and directing the gas flow, including the deagglomerated particles, out of the vortex chamber; wherein the velocity of the gas flow at a distance of 300 mm outside the exit port is less than the velocity of the gas flow at the inlet port.

The invention further provides an arrangement for generating an air flow through a chamber containing powder, so that the powder becomes entrained in the air flow and is carried out of the chamber via an exit port. This involves directing the air flow through the chamber. The chamber has an axis and a wall curved around the axis and the air rotates around this axis. The air flow is also directed through an inlet port of the chamber, wherein the direction of the air flow through the inlet port is tangential to the chamber wall. The direction of the air flow through the exit port is parallel to the axis. A cross-sectional area of the air flow through the chamber is in a normal plane to the air flow and decreases with increasing distance from the inlet port.

In another aspect, an inhaler is provided, for providing the air flow and deagglomeration discussed above. Such inhalers comprise an aerosolising device including a substantially tangential inlet port and a substantially axial exit port. The inhalers also comprise one or more sealed blisters (or capsules) containing the pharmaceutical dry powder composition to be dispensed, and an input device for removably receiving one of these blisters. Upon actuation, the inhaler couples the tangential inlet port with the powder composition in the received blister.

With regard to the aerosolising device, in some embodiments, the aerosolising device is in the form of a vortex chamber of substantially circular cross-section having a substantially tangential inlet port and a substantially axial outlet port. Preferably, the ratio of the diameter of the vortex chamber to the diameter of the exit port is between 4 and 12.

In other embodiments, the aerosolising device is in the form of a vortex chamber of substantially circular cross-section having a substantially tangential inlet port, wherein the inlet port has an outer wall which defines the maximum extent of the inlet port in the radially outward direction of the vortex chamber. The extent of the outer wall in the axial direction of the vortex chamber is substantially equal to the maximum extent of the inlet port in the axial direction of the vortex chamber, the outer wall is substantially parallel with a wall of the vortex chamber.

In other embodiments, the aerosolising device is in the form of a vortex chamber of substantially circular cross-section having a substantially tangential inlet port. A bottom surface defines the furthest extent of the vortex chamber from the exit port in the axial direction, and the bottom surface further defines the furthest axial extent of the inlet port from the exit port.

In yet further embodiments, the aerosolising device is in the form of a vortex chamber of substantially circular cross-section having a substantially tangential inlet port and an inlet conduit arranged to supply a powdered composition entrained in a gas flow to the inlet port, in use, wherein the cross-sectional area of the inlet conduit decreases towards the vortex chamber. The inlet conduit is, upon actuation of the inhaler, coupled to the powder composition in the received blister.

In other embodiments, the aerosolising device is in the form of a vortex chamber of substantially circular cross-section having a substantially tangential inlet port and an arcuate inlet conduit arranged to supply a powdered composition entrained in a gas flow to the inlet port, in use. The inlet conduit is, upon actuation of the inhaler, coupled to the powder composition in the received blister.

In other embodiments, the aerosolising device is in the form of a vortex chamber having an axis defined, at least in part, by a wall which forms a curve about the axis. The vortex chamber has a cross-sectional area in a plane bounded by the axis, and the plane extends in one direction radially from the axis at a given angular position (θ) about the axis. The vortex chamber has a substantially tangential inlet port and a substantially axial exit port, and said cross-sectional area of the vortex chamber decreases with increasing angular position (θ) in the direction, in use, of the gas flow between the inlet port and the outlet port.

In other embodiments, the aerosolising device is in the form of a vortex chamber having an axis defined, at least in part, by a wall which forms a curve about the axis. The vortex chamber has a substantially tangential inlet port and a substantially axial exit port. The vortex chamber is further defined by a base, and the distance (d) between the base and a plane which is normal to the axis and is located on the opposite side of the base to the exit port increase with radial position (r) relative to the axis.

In other embodiments, the aerosolising device includes as chamber defined by a top wall, a bottom wall, and a lateral wall, the lateral wall being curved about an axis which intersects the top wall and the bottom wall. The chamber encloses a cross-sectional area defined by the axis, the top wall, the bottom wall and the lateral wall, and the chamber has an inlet port and an outlet port. The inlet port is tangential to the lateral wall, the outlet port is co-axial with the axis, and the cross-sectional area decreases with increasing angular position from the inlet port in a direction of a gas flow through the inlet port.

In still other embodiments, the aerosolising device is a chamber including a wall, a base, an inlet port and an exit port. The chamber has an axis that is co-axial with the exit port and intersects the base. The wall is curved about the base, the inlet port is tangential to the wall, and a height between the base and a plane normal to the axis at the exit port decreases as a radial position from the axis to the inlet port increases.

One embodiment of the invention is described in detail, by way of example only, with reference to the following drawings:

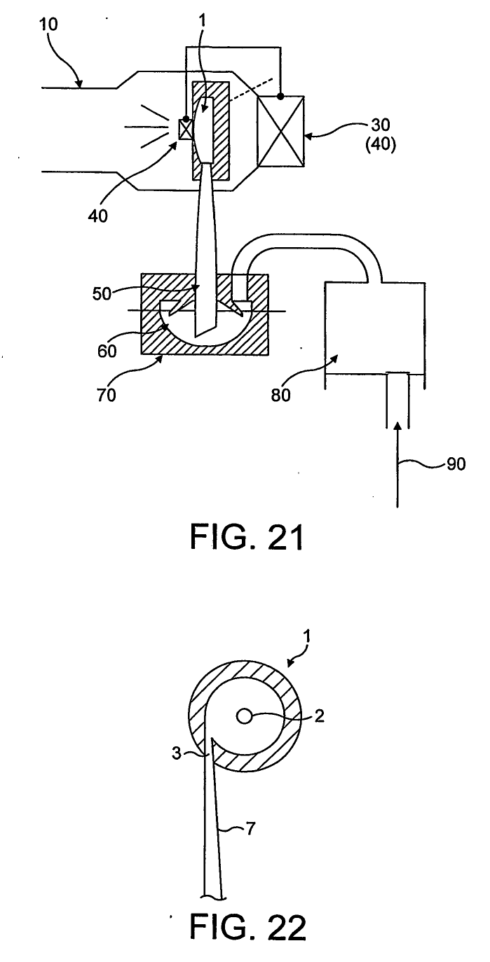

FIG. 21 shows an inhaler and a blister according to the present invention;

FIG. 22 is a top cross-section of a vortex nozzle;

FIG. 23 shows the general form of a vortex chamber of the inhaler shown in FIG. 22;

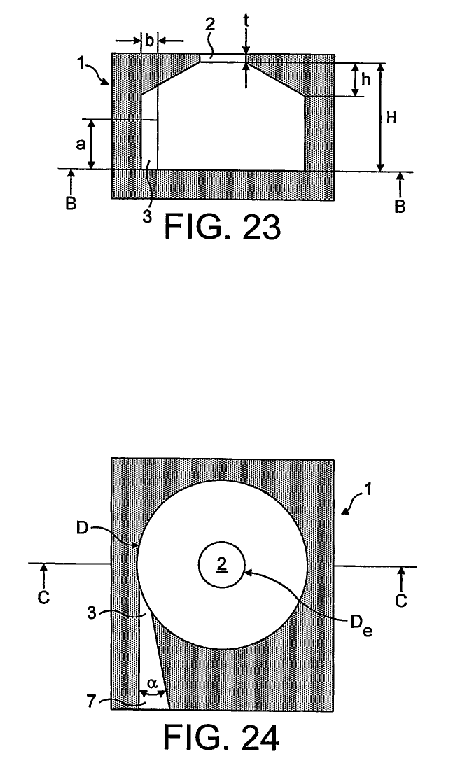

FIG. 24 shows another view of the vortex chamber shown in FIG. 23;

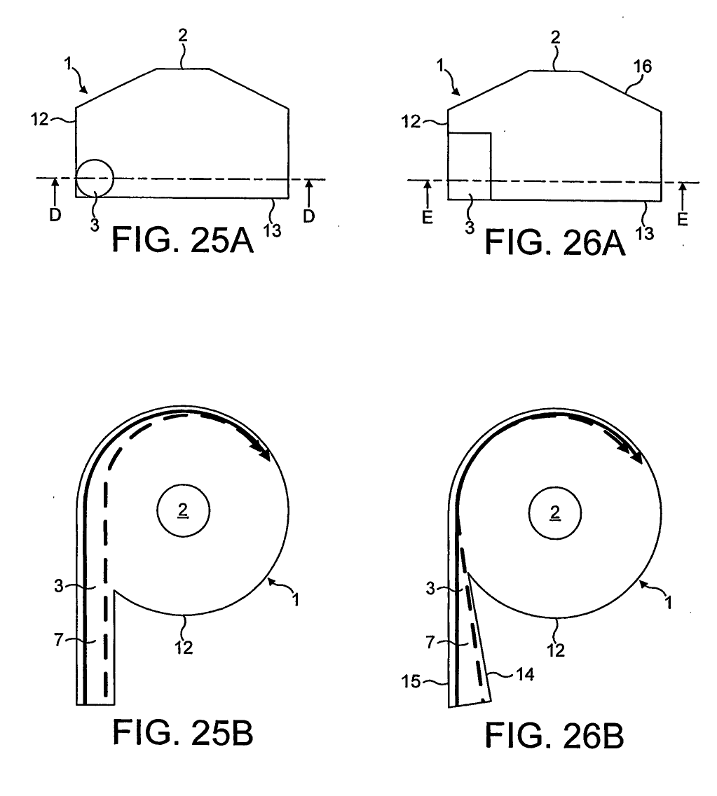

FIG. 25A is a side-view of a vortex chamber with a round inlet port;

FIG. 25B is a sectional view along line D-D of the vortex chamber of FIG. 25A;

FIG. 26A is a side view of a vortex chamber with a rectangular inlet port;

FIG. 26B is a sectional view along line E-E of the vortex chamber of FIG. 26A;

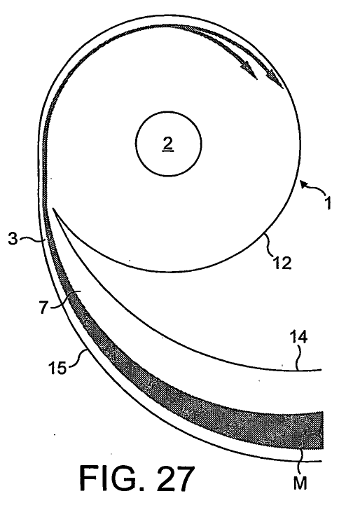

FIG. 27 shows a vortex chamber with an arcuate inlet conduit;

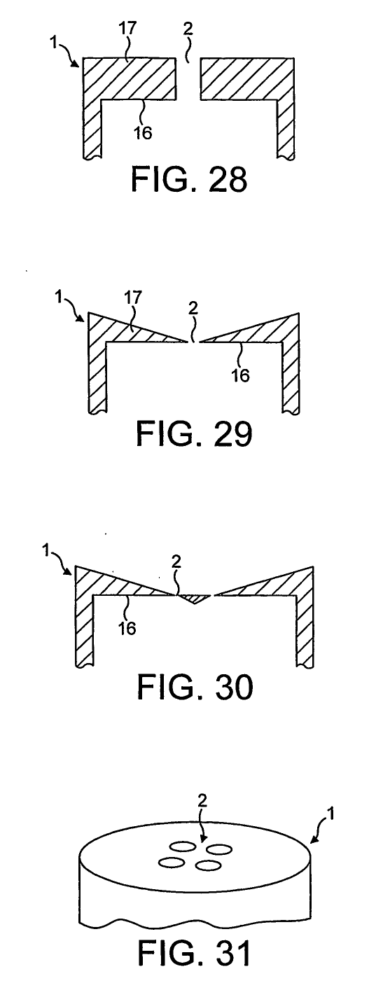

FIGS. 28-31 show detail of embodiments of the exit port of the inhaler in accordance with the invention;

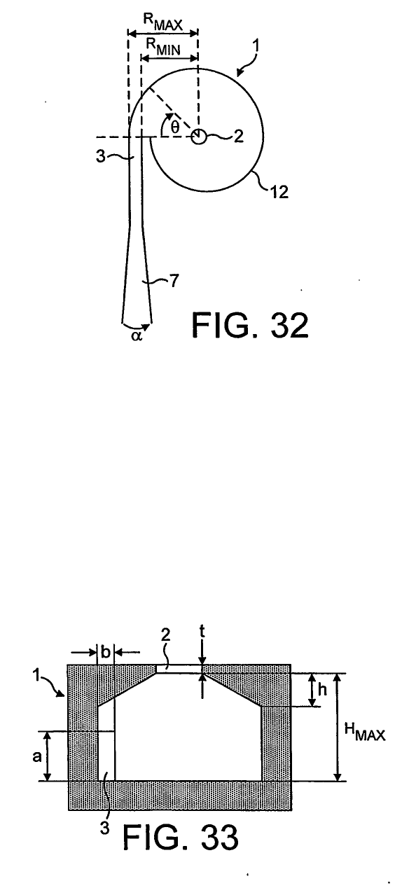

FIG. 32 illustrates as asymmetric vortex chamber in accordance with an embodiment of the invention;

FIG. 33 is a sectional view of a vortex chamber of an asymmetric inhaler in accordance with another embodiment of the invention;

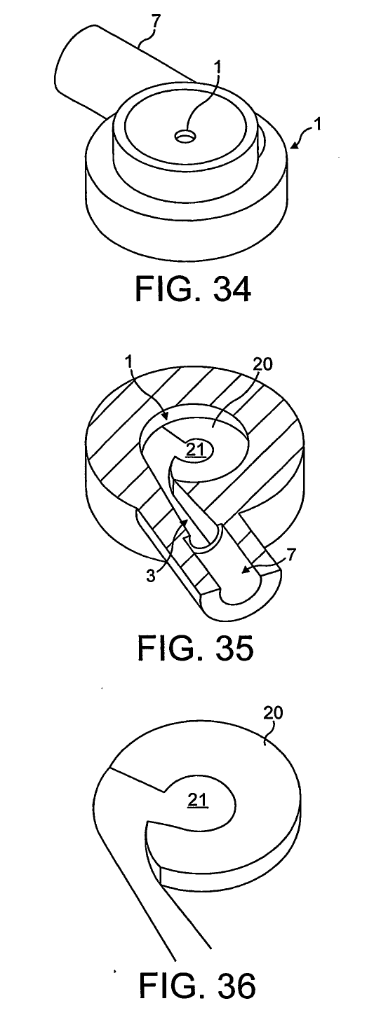

FIG. 34 is a perspective view of a vortex chamber according to FIG. 33;

FIG. 35 is a sectional view of the vortex chamber of FIG. 34;

FIG. 36 is a perspective view of a detail of the vortex chamber of FIGS. 34 and 35;

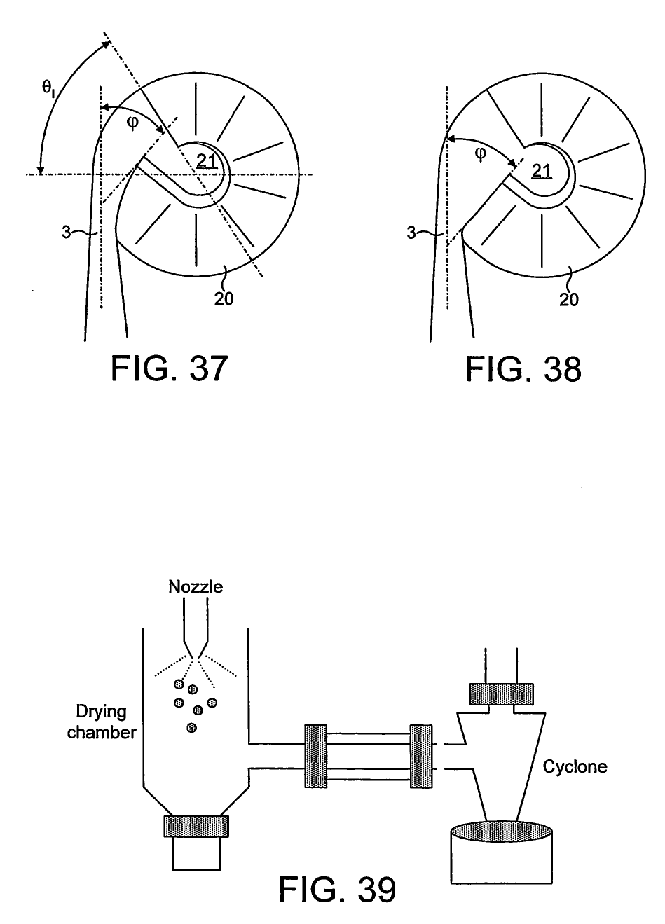

FIG. 37 is a plan view of the detail of FIG. 36; and

FIG. 38 is a plan view of a variation of the detail of FIG. 37.

FIG. 21 shows schematically a preferred inhaler that can be used to deliver a powder formulation to a patient. The inhaler includes a vortex chamber 1 having an exit port 2 and an inlet port 3 for generating an aerosol of the powder formulation. The vortex chamber is situated in a mouthpiece 10 through which the user inhales to use the inhaler. Air passages (not shown) may be defined between the vortex chamber 1 and the mouthpiece 10 so that the user is able to inhale air in addition to the powdered medicament.

The powder formulation is stored in a blister 60 defined by a support 70 and a pierceable foil lid 75. As shown, the support 70 has a cavity formed therein for holding the powder formulation. The open end of the cavity is sealed by the lid 75. An air inlet conduit 7 of the vortex chamber 1 terminates in a piercing head (or rod) 50 which pierces the foil lid 75. A reservoir 80 is connected to the blister 60 via a passage 78. A regulated air supply 90 charges the reservoir 80 with a gas (e.g. air) to a predetermined pressure (e.g. 1.5 bar). Preferably, the blister contains from 1 to 5 mg of powder formulation.

When the user inhales, a valve 40 is opened by a breath-actuated mechanism 30, forcing air from the pressurised air reservoir through the blister 60 where the powdered formulation is entrained in the air flow. The air flow transports the powder formulation to the vortex chamber 1, where a rotating vortex of powder formulation and air is created between the inlet port 3 and the outlet port 2. Rather than passing through the vortex chamber in a continuous manner, the powdered formulation entrained in the airflow enters the vortex chamber in a very short time (typically less than 0.3 seconds and preferably less than 20 milliseconds) and, in the case of a pure drug formulation (i.e. no carrier), a portion of the powder formulation sticks to the walls of the vortex chamber. This powder is subsequently aerosolised by the high shear forces present in the boundary layer adjacent to the powder. The action of the vortex deagglomerates the particles of the powder formulation, or in the case of a formulation comprising a drug and a carrier, strips the drug from the carrier, so that an aerosol of powdered formulation exits the vortex chamber 1 via the exit port 2. The aerosol is inhaled by the user through the mouthpiece 10.

The vortex chamber 1 can be considered to perform two functions: deagglemeration, the breaking up of clusters of particles into individual, respirable particles; and filtration, preferentially allowing particles below a certain size to escape more easily from the exit port 2. Deagglomeration breaks up cohesive clusters of powdered formulation into respirable particles, and filtration increases the residence time of the clusters in the vortex chamber 1 to allow more time for them to be deagglomerated. Deagglomeration can be achieved by creating high shear forces due to velocity gradients in the airflow in the vortex chamber. The velocity gradients are the highest in the boundary area close to the walls of the vortex chamber.

As shown in the detail of FIG. 22, the vortex chamber 1 is in the form of a substantially cylindrical chamber. The vortex chamber has a frustoconical portion in the region of the exit port 2. The inlet port 3 is substantially tangential to the perimeter of the vortex chamber and the exit port is generally concentric with the axis of the vortex chamber. Thus, gas enters the vortex chamber tangentially via the inlet port 3 and exits axially via the exit port 2. Between the inlet port 3 and the exit port 2, a vortex is created in which shear forces are generated to deagglomerate the particles of medicament. The length of the exit port 2 is preferably minimised to reduce the possibility of deposition of the active agent in the walls of the exit port 2.

The ratio of the diameter of the vortex chamber to the diameter of the exit port can be significant in maximising the fine particle fraction of the active agent aerosol which is expelled from the exit port. Thus, the ratio of the diameter of the vortex chamber to the diameter of the exit port is preferably between 4 and 12. It has been found that when the ratio is between 4 and 12 the proportion of the particles of the powdered medicament with an effective diameter in the range of 1-3 μm is maximised. For an enhanced FPF, the ratio is preferably greater than 5, more preferably greater than 6 and preferably less than 9, most preferably less than 8. In the preferred arrangement the ration is 7:1.

In certain embodiments of the invention, the diameter if the vortex chamber is between 2 and 12 mm. The diameter of the vortex chamber is preferably greater than 4 mm, more preferably at least 5 mm and preferably less than 8 mm, more preferably less than 6 mm. In the preferred embodiment, the diameter of the vortex chamber is 5 mm. In these embodiments, the height of the vortex chamber is generally between 1 and 8 mm. The height of the vortex chamber is preferably less than 4 mm and more preferably less than 2 mm. In the preferred embodiment, the height of the vortex chamber is 1.6 mm.

In general, the vortex chamber is substantially cylindrical. However, the chamber may take other forms. For example, the vortex chamber may be frustoconical. Where the diameter of the vortex chamber or the exit port id not constant along its length, the ratio of the largest diameter of the vortex chamber to the smallest diameter of the exit port should be within the range specified above.

The aerosolising device comprises an exit port, for example as described above. The diameter of the exit port is generally between 0.5 and 2.5 mm. The diameter of the exit port is preferably greater than 0.6 mm and preferably less than 1.2 mm, more preferably less than 1.0 mm. In a preferred embodiment, the diameter of the exit port is 0.7 mm.

TABLE 8

| Dimension | | Preferred Value |
|---|---|---|
| D | Diameter of chamber | 5.0 mm |
| H | Height of chamber | 1.6 mm |
| h | Height of conical part of chamber | 0.0 mm |
| Dₑ | Diameter of exit port | 0.7 mm |
| t | Length of exit port | 0.3 mm |
| a | Height of inlet port | 1.1 mm |
| b | Width of inlet port | 0.5 mm |
| α | Taper angle of inlet conduit | 12° |

FIGS. 23 and 24 show the general form of the vortex chamber of the inhaler in FIG. 1. The geometry of the vortex chamber is defined by the dimensions listed in Table 8. The preferred values of these dimensions are also listed in Table 8. It should be noted that the height h of the conical part of the chamber is 0 mm, because it has been found that the vortex chamber functions most efficiently when the top of the chamber is flat.

As shown in Table 9 below, the proportion of the particles of active agent emitted in the aerosol having an effective particle diameter of less than 6.8 μm generated by the vortex chamber (the 6.8 μm particle fraction) depends on the ratio of the diameters of the chamber (D) and the exit port (Dₑ) The normalised average 6.8 μm particle fraction of the powdered active agent loaded into the inhaler. The active agent used was pure Intal™ sodium cromoglycate (Fisons, UK).

TABLE 9

| | Average particle | Normalised average |
|---|---|---|
| | fraction < | particle fraction < |
| Ratio D/Dₑ | 6.8 μm (%) | 6.8 μm (%) |
| 2.0 | 64.7 | 73.1 |
| 3.1 | 70.8 | 79.9 |
| 4.0 | 75.5 | 85.2 |
| 6.0 | 81.0 | 91.4 |
| 7.1 | 83.5 | 94.3 |
| 8.0 | 83.2 | 93.9 |
| 8.6 | 80.6 | 91.0 |

From Table 9, it can be seen that where the ratio of the diameters of the vortex chamber and the exit port is 4 or more, the normalised 6.8 μm particle fraction is over 850%. Thus, the deagglomeration efficiency of the vortex chamber is significantly improved where the ratio is in this range. With the preferred ratio of 7.1, a normalised 6.8 μm particle fraction of 94.3% is achieved.

FIGS. 25A and 25B show a vortex chamber 1 in which the inlet port 3 has a circular cross-section. As represented by the solid arrow in FIG. 25B, a portion of the airflow entering the vortex chamber via the inlet port 3 follows the lateral wall 12 of the vortex chamber 1. The powder entrained in this airflow is therefore introduced directly into the airflow at the boundary layer adjacent to the lateral wall 12 of the vortex chamber, where the velocity gradient in the radial direction is at a maximum. The maximal velocity gradient results in maximal shear forces on the agglomerated particles of the powder and thus maximum deagglomeration.

However, as represented by the dashed arrow in FIG. 25B, a portion of the airflow entering the vortex chamber via the inlet port 3 does not follow the chamber wall 12, but rather crosses the chamber and meets the wall 12 at a point opposite the inlet port 3. At this point, there is increased turbulence, because the is flow must make an abrupt change of direction. This turbulence disturbs the boundary layer adjacent the wall of the chamber and thereby reduces the effectiveness of the deagglomeration of the powder.

FIGS. 26A and 26B show a vortex chamber 1 in which the inlet chamber has a rectangular cross-section. The rectangular cross-section maximises the length of the perimeter of the inlet port that is coincident with the wall 12 of the chamber, such that the maximum air flow is introduced into the boundary layer of the vortex. Similarly, the rectangular cross-section maximises the width of the perimeter of the inlet port 3 that is coincident with the bottom surface 13 of the vortex chamber. In this way, deposition of powder in the vortex chamber 1 is prevented, because the vortex occupies the entire chamber.

In addition to having a rectangular cross-section, the inlet port 3 of FIGS. 26A and 26B is supplied by an inlet conduit 7 which tapers towards the vortex chamber 1. Thus, the inlet conduit is defined by an inner wall 14 and an outer wall 15. The outer wall is substantially tangential to the wall 12 of the vortex chamber 1. The spacing of the inner wall 14 from the outer wall 15 decreases towards the vortex chamber 1, so that the inner wall 14 urges the air flow into the vortex chamber 1 towards the boundary layer.

Furthermore, the decreasing cross-sectional area of the inlet conduit 7 causes the flow velocity to increase, thereby reducing deposition of powder on the way to the vortex chamber 1.

As indicated by the arrows in FIG. 26B, all of the airflow entering the vortex chamber via the inlet port 3 follows the wall of the chamber 12. The powder entrained in this airflow is therefore introduced directly into the airflow at the boundary layer adjacent the wall 12 of the chamber, and the deagglomeration is maximised.

FIGS. 28 to 31 show various options for the exit port 2 of the vortex chamber. The characteristics of the exit plume of the aerosol are determined, at least in part, by the configuration of the exit port 2. For example, if the aerosol leaves an exit port 2 of 1 mm diameter at a flow rate of 2 litres per minute, the velocity at the exit port will be approximately 40 m/s. This velocity can be reduced to a typical inhalation velocity of 2 m/s within a few centimetres of the chamber or nozzle by providing a strongly divergent aerosol plume.

In FIG. 28, the exit port 2 is a simple orifice defined through the upper wall 17 of the vortex chamber. However, the thickness of the upper wall 17 means that the exit port 2 has a length which is greater than its diameter. Thus, there is a risk of deposition in the exit port as the aerosol of powder exits. Furthermore, the tubular exit port tends to reduce the divergence of the exit plume. These problems are solved in the arrangement of FIG. 29, by tapering the upper wall 17 of the vortex chamber 1 towards the exit port 2, so that the exit port 2 is defined by a knife edge of negligible thickness. For an exit port with a diameter of 1 mm, an exit port length of 2.3 mm gives a plume angle of 60°, whereas reducing this length to 0.3 mm increases the angle to 90°.

In FIG. 30, the exit port is annular and is also defined by a knife edge. This arrangement produces an exit plume that slows down more quickly than a circular jet, because the annular exit port has a greater perimeter than a circular port of the same diameter and produces a jet that mixes more effectively with the surrounding static air.

In FIG. 31, multiple orifices form the exit port 2 and produce a number of smaller plumes which break up and slow down in a shorter distance than a single large plume.

FIG. 27 shows an embodiment of the vortex chamber 1 in which the inlet conduit 7 is arcuate and tapers towards the vortex chamber. As shown by the arrows in FIG. 33, the arcuate inlet conduit 7 urges the entrained particles of the powdered formulation to wards the outer wall 15 of the inlet conduit 7. In this way, when the powder enters the vortex chamber through the inlet port 3, the powder is introduced directly into the boundary layer next to the wall 12 of the vortex chamber 1, where the shear forces are at a maximum. In this way, improved deagglomeration is achieved.

The inhaler in accordance with some embodiments of the invention is able to generate a relatively slow moving aerosol with a high fine particle fraction. The inhaler is capable of providing complete and repeatable aerosolisation of a measured dose of powdered active agent and of delivering the aerosolised dose into the patient's inspiratory flow at a velocity of less than or equal to the velocity of the inspiratory flow, thereby reducing deposition by impaction in the patient's mouth. Furthermore, the efficient aerosolising system allows for a simple, small and low cost device, because the energy used to create the aerosol is small. The fluid energy required to create the aerosol can be defined as the integral over time of the pressure multiplied by the flow rate. This is typically less than 5 joules and can be as low as 3 joules.

It is clear that similar effects can be achieved using asymmetric inhalers. In such inhalers, the vortex chamber has an asymmetric shape.

In the embodiment shown in FIG. 32, the wall 12 of the vortex chamber 1 is in the form of a spiral or scroll. The inlet port 3 is substantially tangential to the perimeter if the vortex chamber 1 and the exit port 2 is generally concentric with the axis of the vortex chamber 1.

Thus, the gas enters the vortex chamber tangentially via the inlet port 3 and exits axially via the exit port 2. The radius R of the vortex chamber measured from the centre of the exit port 2 decreases smoothly from a maximum radius Rₘₐₓ at the inlet port to a minimum radius Rₘᵢₙ. Thus, the radius R at an angle of θ from the position of the inlet port 3 is given by R=Rₘₐₓ(1−θk/2π) where k=(Rₘₐₓ−Rₘᵢₙ)/Rₘₐₓ.

The effective radius of the vortex chamber decreases as the air flow and entrained particles of active agent circulate around the chamber. In this way, the effective cross-sectional area of the vortex chamber 1 experienced by the air flow decreases, so that the air flow is accelerated and there is reduced deposition of the entrained particles of active agent. In addition, when the flow of air has gone through 2π radians (360°), the air flow is parallel to the incoming airflow through the inlet port 3, so that there is a reduction in the turbulence caused by the colliding flows.

Between the inlet port 3 and the exit port 2, a vortex is created in which shear forces are generated to deagglomerate the particles of the powdered formulation. As discussed above, the length of the exit port 2 is preferably as short as possible, to reduce the possibility of deposition of the drug on the walls of the exit port 2.

FIG. 33 shows the general form of the vortex chamber of the inhaler of FIG. 32. The geometry of the vortex chamber is defined by the dimensions listed in Table 10. The preferred values of these dimensions are also listed in Table 10. It should be noted that the height of the conical part of the chamber is 0 mm, because it has been found that the vortex chamber functions most efficiently when the top (roof 16) of the chamber is flat.

TABLE 10

| Dimension | | Preferred Value |
|---|---|---|
| Rₘₐₓ | Maximum radius of chamber | 2.8 mm |
| Rₘᵢₙ | Minimum radius of chamber | 2.0 mm |
| Hₘₐₓ | Maximum height of chamber | 1.6 mm |
| h | Height of conical part of chamber | 0.0 mm |
| Dₑ | Diameter of exit port | 0.7 mm |
| t | Length of exit port | 0.3 mm |
| a | Height of inlet port | 1.1 mm |
| b | Width of inlet port | 0.5 mm |
| α | Taper angle of inlet conduit | 9°, then 2° |

The 6.8 μm particle fraction of the aerosol generated by the vortex chamber 1 according to FIG. 32 is improved relative to a circular vortex chamber (as shown in FIGS. 21-31).

FIGS. 34 to 38 show another asymmetric inhaler in accordance with the present invention in which the vortex chamber 1 includes a ramp 20 which reduces the height of the vortex chamber 1 from the bottom up with increasing angular displacement θ from the inlet port 3. A substantially circular region 21 in the centre of the vortex chamber 1 remains flat.

Particle Cohesiveness

For formulations to reach the deep lung or the blood stream via inhalation, the active agent in the formulation must be in the form of very fine particles, for example, having a mass median aerodynamic diameter (MMAD) of less than 10 μm. It is well established that particles having an MMAD of greater than 10 μm are likely to impact on the walls of the throat and generally do not reach the lung. Particles having an MMAD in the region of 5 μm to 2 μm will generally be deposited in the respiratory bronchioles whereas particles having an MMAD in the range of 3 to 0.05 μm are likely to be deposited in the alveoli or be absorbed into the bloodstream.

Preferably, for delivery to the lower respiratory tract or deep lung, the MMAD of the active particles is not more than 10 μm, and preferably not more than 5 μm, more preferably not more than 3 μm, and may be less than 1 μm. Ideally, at least 90% by weight of the active particles in a dry powder formulation should have an MMAD of not more than 10 μm, preferably not more than 5 μm, more preferably not more than 3 μm and most preferably not more than 1 μm.

When dry powders are produced using conventional processes, the active particles will vary in size, and often this variation can be considerable. This can make it difficult to ensure that a high enough proportion of the active particles are of the appropriate size for administration to the correct site. It is therefore desirable to have a dry powder formulation wherein the size distribution of the active particles is as narrow as possible. This will improve dose efficiency and reproducibility.

Fine particles, that is, those with an MMAD of less than 10 μm, are thermodynamically unstable due to their high surface area to volume ratio, which provides a significant excess surface free energy and encourages the particles to agglomerate. In the inhaler, agglomeration of fine particles and adherence of such particles to the walls of the inhaler are problems that result in the fine particles leaving the inhaler as large, stable agglomerates, or being unable to leave the inhaler and remaining adhered to the interior of the inhaler, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung.

The tendency of fine particles to agglomerate means that the FPF of a given dose is highly unpredictable and a variable proportion of the fine particles will be administered to the lung, or to the correct part of the lung, as a result.

In an attempt to improve this situation and to provide a consistent FPF and FPD, dry powder formulations often include additive material.

The additive material is intended to decrease the cohesion between particles in the dry powder formulation. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, whereupon the particles are expelled from the device and inhaled. As the agglomerates break up, the active particles return to the form of small individual particles which are capable of reaching the lower lung.

In the prior art, dry powder formulations are discussed which include distinct particles of additive material (generally of a size comparable to that of the fine active particles). In some embodiments, the additive material may form a coating, generally a discontinuous coating, on the active particles and/or any carrier particles.

Preferably, the additive material is an anti-adherent material and it will tend to reduce the cohesion between particles and will also prevent fine particles becoming attached to the inner surfaces of the inhaler device. Advantageously, the additive material is an anti-friction agent or glidant and will give better flow of the pharmaceutical composition in the inhaler. The additive materials used in this way may not necessarily be usually referred to as anti-adherents or anti-friction agents, but they will have the effect of decreasing the cohesion between the particles or improving the flow of the powder. The additive materials are often referred to as force control agents (FCAs) and they usually lead to better dose reproducibility and higher fine particle fractions.

Therefore, an FCA, as used herein, is an agent whose presence on the surface of a particle can modify the adhesive and cohesive surface forces experienced by that particle, in the presence of other particles. In general, its function is to reduce both the adhesive and cohesive forces.

In general, the optimum amount of additive material to be included in a dry powder formulation will depend on the chemical composition and other properties of the additive material and of the active material, as well as upon the nature of other particles such as carrier particles, if present. In general, the efficacy of the additive material is measured in terms of the fine particle fraction of the composition.

Known additive materials usually consist of physiologically acceptable material, although the additive material may not always reach the lung, for example where the additive particles are attached to the surface of carrier particles so that they will generally be deposited, along with those carrier particles, at the back of the throat of the user.

Preferred additive materials for used in prior art dry powder formulations include amino acids, peptides and polypeptides having a molecular weight of between 0.25 and 1000 kDa and derivatives thereof, dipolar ions such as zwitterions, phospholipids such as lecithin, and metal stearates such as magnesium stearate.

In a further attempt to improve this situation and to provide a consistent FPF and FPD, dry powder formulations often include coarse carrier particles of excipient material mixed with fine particles of active material. Rather that sticking to one another, the fine active particles tend to adhere to the surfaces of the coarse carrier particles whilst in the inhaler device, but are supposed to release and become dispersed upon actuation of the dispensing device and inhalation into the respiratory tract, to give a fine suspension. The carrier particles preferably have MMADs greater than 90 μm.

The inclusion of coarse carrier particles is also very attractive where very small doses of active agent are dispensed. It is very difficult to accurately and reproducibly dispense very small quantities of powder and small variations in the amount of powder dispensed will mean large variations in the dose of active agent where the powder comprises mainly active particles. Therefore, the addition of a diluent, in the form of large excipient particles will make dosing more reproducible and accurate.

Carrier particles may be of any acceptable excipient material or combination of materials. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously the carrier particles are of a polyol. In particular the carrier particles may be particles of crystalline sugar, for example mannitol, dextrose or lactose. Preferably, the carrier particles are of lactose.

Advantageously, substantially all (by weight) of the carrier particles have a diameter which lies between 20 μm and 1000 μm, more preferably 50 μm and 1000 μm.

Preferably, the diameter of substantially all (by weight) of the carrier particles is less than 355 μm and lies between 20 μm and 250 μm.

Preferably at least 90% by weight of the carrier particles have a diameter between from 60 μm to 180 μm. The relatively large diameter of the carrier particles improves the opportunity for other, smaller particles to become attached to the surfaces of the carrier particles and to provide good flow and entrainment characteristics and improved release of the active particles in the airways to increase deposition of the active particles in the lower lung.

The ratios in which the carrier particles (if present) and composite active particles are mixed will, of course, depend on the type of inhaler device used, the type of active particles used and the required dose. The carrier particles may be present in an amount of at least 50%, more preferably 70%, advantageously 90% and most preferably 95% based on the combined weight of the composite active particles and the carrier particles.

However, a further difficulty is encountered when adding coarse carrier particles to a composition of fine active particles and that difficulty is ensuring that the fine particles detach from the surface of the large particles upon actuation of the delivery device.

The step of dispersing the active particles from other active particles and from carrier particles, if present, to form an aerosol of fine active particles for inhalation is significant in determining the proportion of the dose of active material which reaches the desired site of absorption in the lungs. In order to improve the efficiency of that dispersal it is known to include in the composition additive materials of the nature discussed above. Compositions comprising fine active particles and additive materials are disclosed in WO 97/03649 and WO 96/23485.

In light of the foregoing problems associated with known dry powder formulations, even when including additive material and/or carrier particles, it is an aim of the present invention to provide dry powder compositions which have physical and chemical properties which lead to an enhanced FPF and FPD. This leads to greater dosing efficiency, with a greater proportion of the dispensed active agent reaching the desired part of the lung for achieving the required therapeutic effect.

It is highly desirable to be able to prepare fine particles comprising an active agent using simple methods and simple apparatus. As discussed below, dry powder formulations can be prepared, without requiring elaborate, multi-step methods, wherein the active particle have an MMAD suitable for deposition in the deep lung and wherein the dry powder formulations exhibit the preferred FPF and FPD discussed above, regardless of the type of device used to dispense them.

Known additive materials or force control agents (FCAs) usually consist of physiologically acceptable material, although the FCAs may not always reach the lung. For example, where additive particles are attached to the surface of carrier particles, they will generally be deposited, along with those carrier particles, at the back of the throat of the user.

Advantageously, the FCA includes one or more compounds selected from amino acids and derivatives thereof, and peptides and derivatives thereof. Amino acids, peptides and derivatives of peptides are physiologically acceptable and give acceptable release of the active particles on inhalation.

It is particularly advantageous for the FCA to comprise an amino acid. The FCA may comprise one or more of any of the following amino acids: leucine, isoleucine, lysine, valine, methionine, and phenylalanine. The FCA may be a salt or a derivative of an amino acid, for example aspartame or acesulfame K. Preferably, the FCA consists substantially of an amino acid, more preferably of leucine, advantageously L-leucine. The D- and DL-forms may also be used. As indicated above, leucine has been found to give particularly efficient dispersal of the active particles on inhalation.

The FCA may include one or more water soluble substances. This helps absorption of the FCA by the body if it reaches the lower lung. The FCA may include dipolar ions, which may be zwitterions. It is also advantageous to include a spreading agent as an FCA, to assist with the dispersal of the composition in the lungs. Suitable spreading agents include surfactants such as known lung surfactants (e.g. ALEC,™) which comprise phospholipids, for example, mixtures of DPPC (dipalmitoyl phosphatidylcholine) and PG (phosphatidylglycerol). Other suitable surfactants include, for example, dipalmitoyl phosphatidylethanolamine (DPPE), dipalmitoyl phosphatidylinositol (DPPI).

The FCA may comprise a metal stearate, or a derivative thereof, for example, sodium stearyl fumarate or sodium stearyl lactylate. Advantageously, it comprises a metal stearate. For example, zinc stearate, magnesium stearate, calcium stearate, sodium stearate or lithium stearate. Preferably, the additive material comprises magnesium stearate.

The FCA may include or consist of one or more surface active materials, in particular materials that are surface active in the solid state, which may be water soluble or water dispersible, for example lecithin, in particular soya lecithin, or substantially water insoluble, for example solid state fatty acids such as oleic acid, lauric acid, palmitic acid, stearic acid, etucic acid, behenic acid, or derivatives (such as esters and salts) thereof such as glyceryl behenate. Specific examples of such materials are phosphatidylcholines, phosphatidylethanolamines, phosphatidylglycerols and other examples of natural and synthetic lung surfactants; lauric acid and its salts, for example, sodium lauryl sulphate, magnesium lauryl sulphate; triglycerides such as Dynsan 118 and Cutina HR; and sugar esters in general. Alternatively, the FCA may be cholesterol.

Other possible FCAs include sodium benzoate, hydrogenated oils which are solid at room temperature, talc, titanium dioxide, aluminium dioxide, silicon dioxide and starch. Also useful as FCAs are film-forming agents, fatty acids and their derivatives, as well as lipids and lipid-like materials.

In one embodiment of the invention, the FCA comprises an amino acid, a derivative of an amino acid, a metal stearate or a phospholipid. Preferably, the FCA comprises one or more of L-, D- or DL-forms of leucine, isoleucine, lysine, valine, methionine, phenylalanine, or Aerocine, lecithin or magnesium stearate. In another embodiment, the FCA comprises leucine and preferably 1-leucine.

In some embodiments, a plurality of different FCAs can be used.

As touched upon above, for the best powder performance, the powder formulations of the present invention need to exhibit particle cohesiveness which is tailored to the type of device being used to dispense it. Where the device is efficient at extracting the powder from the device, such as is this case with active dispensing devices such as Aspirair™, the powder formulation preferably exhibits a degree of cohesiveness in order to retard the expulsion of the powder from the device. This, in turn, has a beneficial effect on the plume dynamics, leading to reduced deposition of the powder in the throat.

The discussions below look at different approaches to particle engineering, allowing one to control and refine the particle cohesion, so that ideal powder behaviour and performance can be achieved and this can be matched to the device to be used to dispense the powder.

Spray Dried Powder Particles

In particular, the present invention seeks to optimise the preparation of particles of active agent used in the dry powder composition by engineering the particles making up the dry powder composition and, in particular, by engineering the particles of active agent. It is an aim of the present invention to provide particles of active agent which are smaller than those produced by known methods or processes. It is also an aim to provide particles with a particle make-up and morphology which will produce high FPF and FPD results.

Whilst the FPF and FPD of a dry powder formulation are dependent on the nature of the powder itself, these values ate also influenced by the type of inhaler used to dispense the powder. For example, the FPF obtained using a passive device will tend not to be as good as that obtained with the same powder but using an active device, such as an Aspirair™ device (see WO 01/00262 and GB2353222).

It is an aim of the present invention to optimise the powder properties, so that the FPF and FPD are improved compared to those obtained using known powder formulations, regardless of the type of device used to dispense the composition of the invention.

It is a particular aim of the present invention to provide a dry powder formulation which has an FPF of at least 50%. Preferably, the FPF(ED) will be between 70 and 990%, more preferably between 80 and 99% Furthermore, it is desirable for the FPF(MD) to be at least 50%. Preferably, the FPF(MD) will be between 50 and 99%, more preferably between 60 and 99%.

The engineering of spray dried particles according to the present invention is described below in detail, with reference to the following drawings:

FIG. 39 shows a schematic set-up of a conventional type spray drying apparatus with a 2-fluid nozzle;

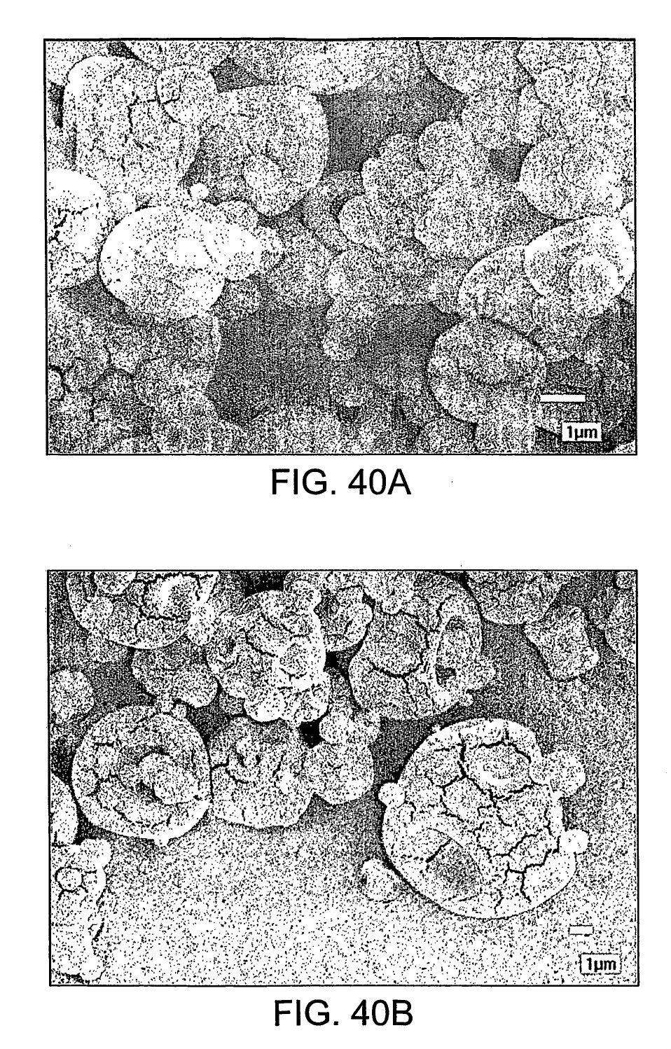

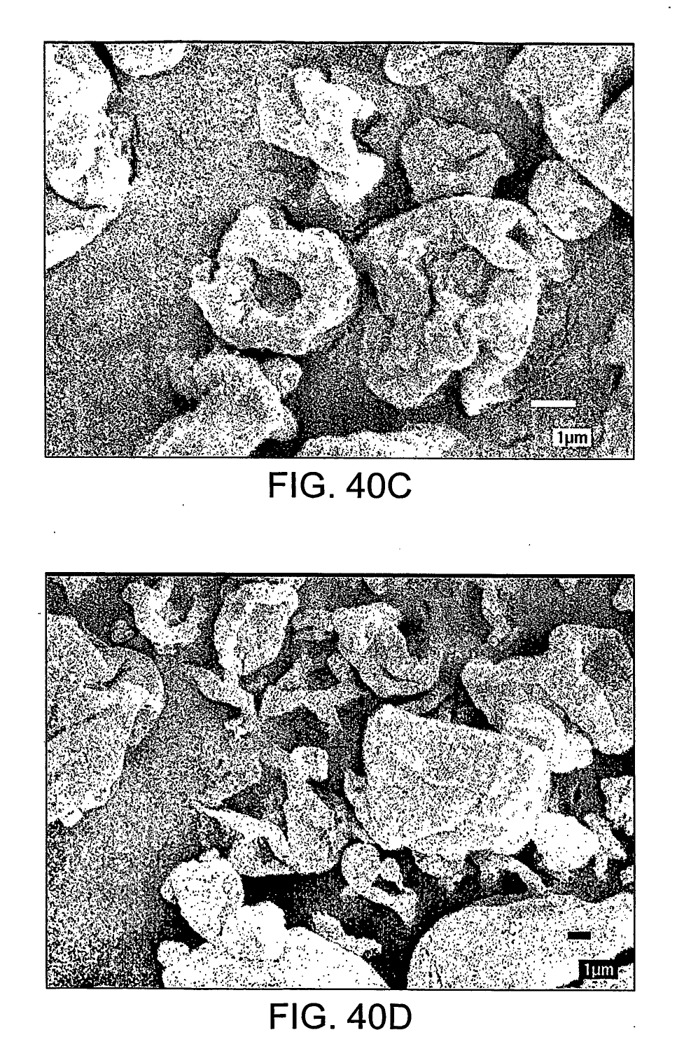

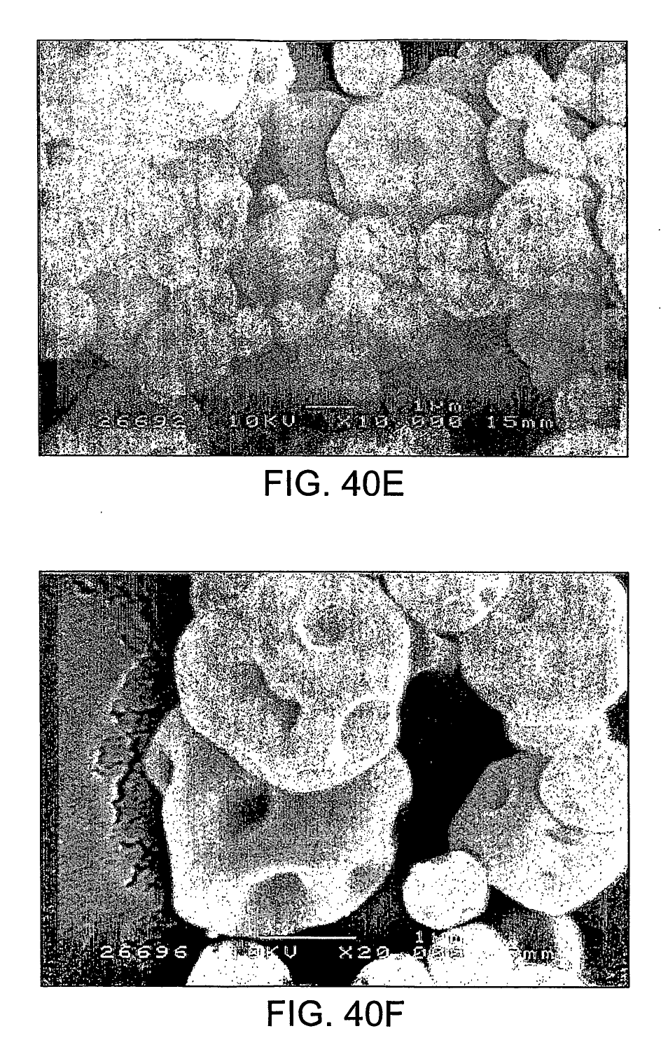

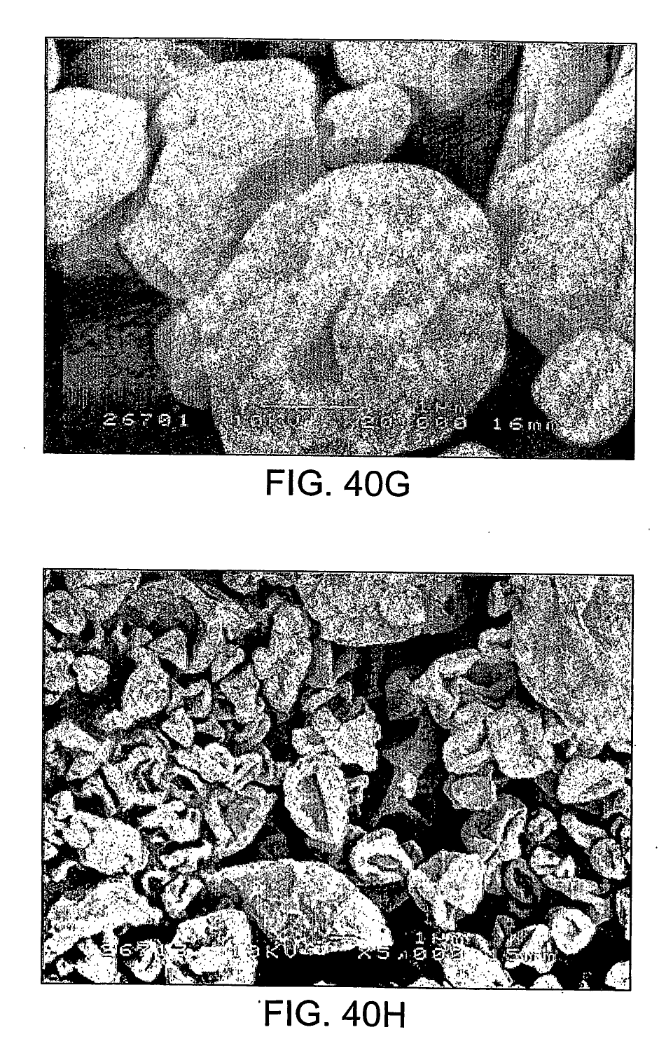

FIGS. 40A-40D are SEM micrographs of 2-fluid nozzle spray dried powders which were co-spray dried with increasing amounts of 1-leucine (0%, 5%, 25% and 50% w/w), without secondary drying;

FIGS. 40E-40H are SEM micrographs of 2-fluid nozzle spray dried powders which were co-spray dried with increasing amounts of 1-leucine (2%, 5%, 10% and 50% w/w), after secondary drying;

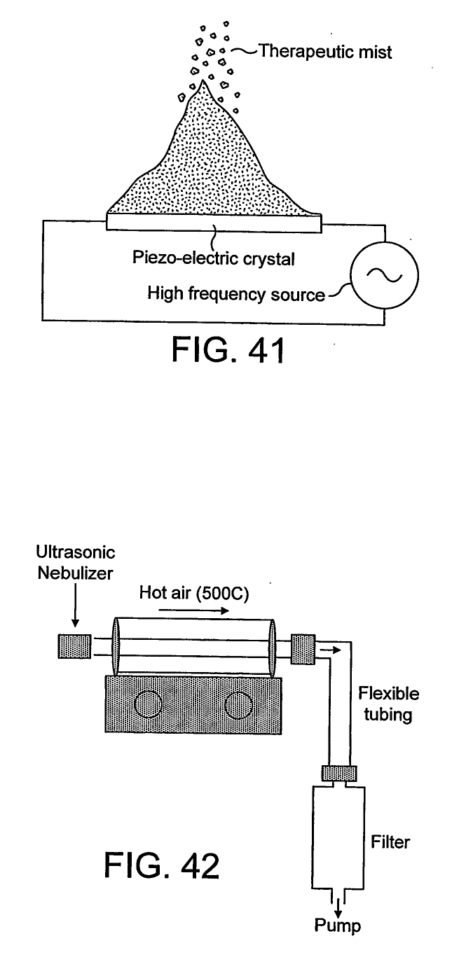

FIG. 41 shows a schematic diagram of an ultrasonic nebuliser producing fine droplets;

FIG. 42 shows a schematic set-up of a spray drier incorporating an ultrasonic nebuliser;

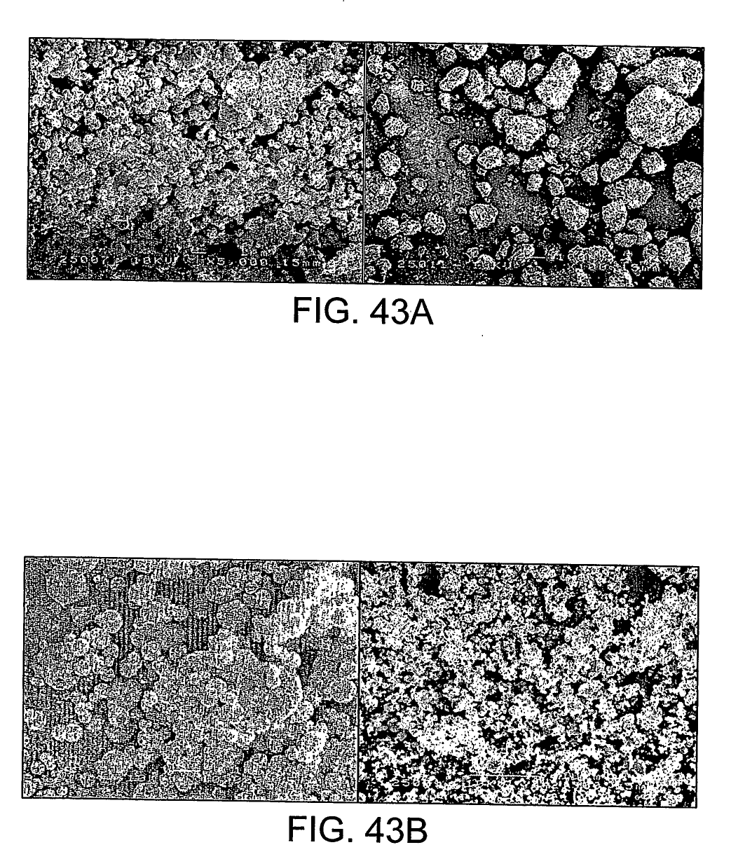

FIGS. 43A and 43B show SEM micrographs of spray dried nebulised heparin alone and with 10% w/w leucine, without secondary drying;

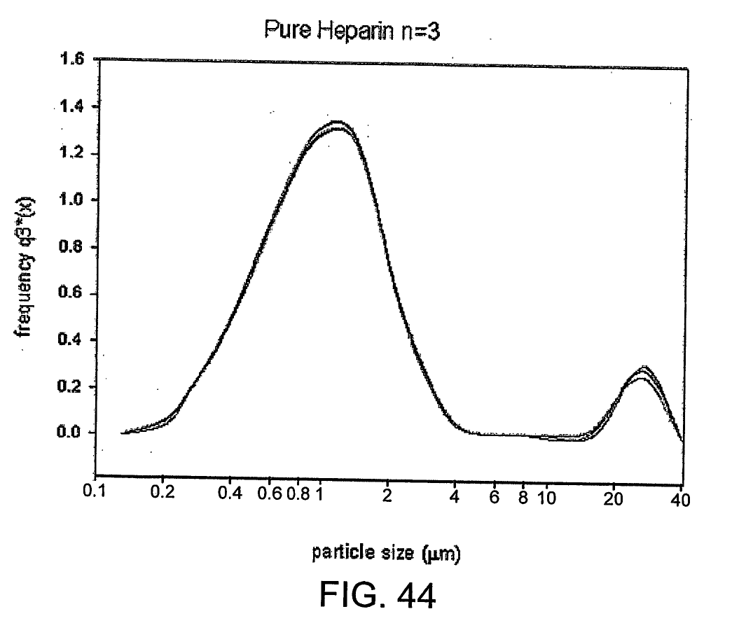

FIG. 44 shows a typical size distribution curve of three repeated tests of spray dried nebulised heparin (with no FCA);

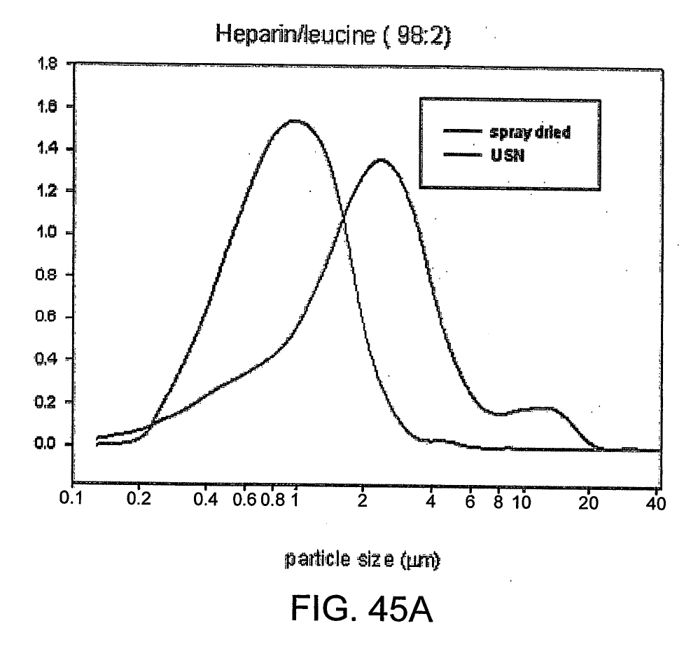

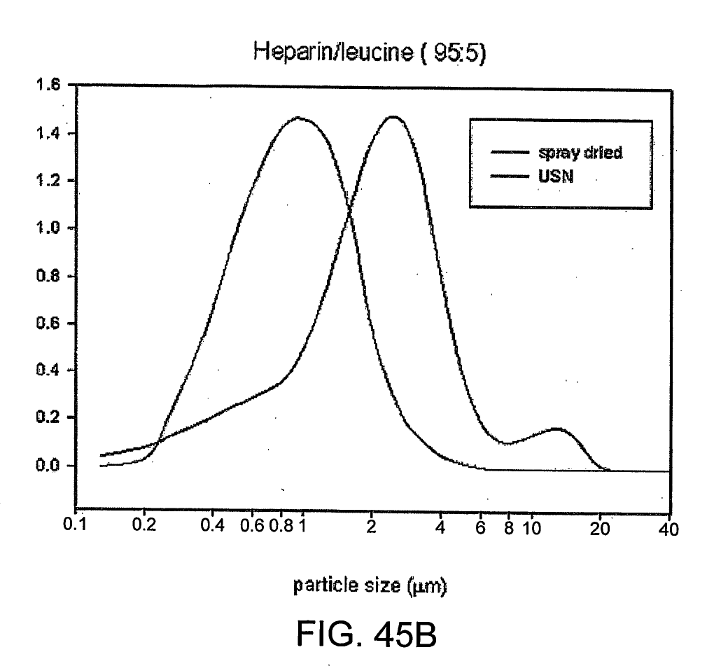

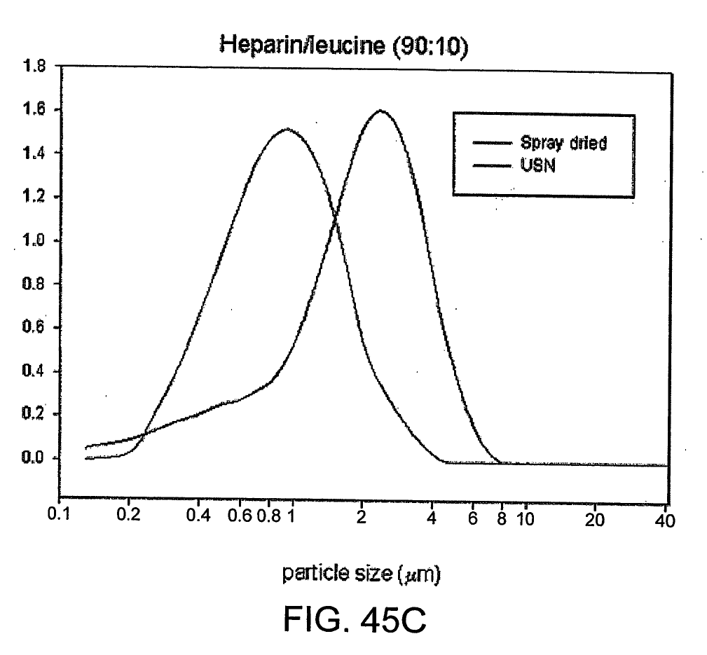

FIGS. 45A-45C show a comparison between particle size distribution curves of 2-fluid nozzle spray dried powders and ultrasonic nebulised powders comprising a blend of heparin and leucine (2% w/w, 5% w/w and 10% w/w); and

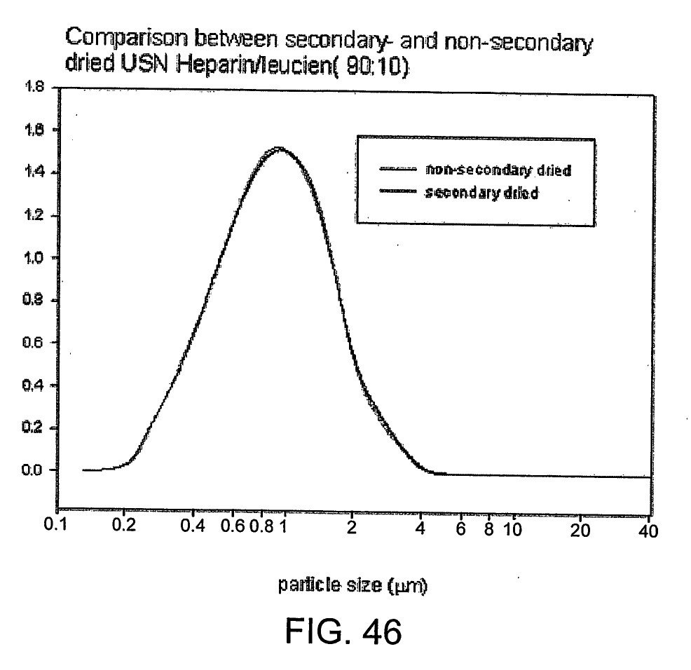

FIG. 46 shows a comparison between particle size distribution curves of secondary dried and not secondary dried powders. The powder used was heparin with leucine (10% w/w).

In the past, two basic methods have been used to make fine particles of active material. Firstly, the material is ground or milled to form particles with the desired size. Alternatively, the particles may be made by spray drying techniques.

The present invention is concerned with improving the conventional spray drying techniques, in order to produce active particles with enhanced chemical and physical properties so that they perform better when dispensed from a DPI than particles formed using conventional spray drying techniques. The improved results are preferably achieved regardless of whether the DPI used to dispense the powder is an active inhaler or a passive inhaler.

Spray drying is a well-known and widely used technique for producing particles of material. To briefly summarise, the material to be made into particles is dissolved or dispersed in a liquid or can be made into a liquid which is sprayed through a nozzle under pressure to produce a mist or stream of fine droplets. These fine droplets are usually exposed to heat which rapidly evaporates the moisture in the droplets, leaving dry powder particles. The process is relatively cheap and simple.

A standard method for producing particles of an active material involves using a conventional spray dryer, such as a Büchi B-191 under a “standard” set of parameters. Such standard parameters are set out in Table 11.

TABLE 11
“Standard” parameters used in spray drying using the
Büchi B-191 spray dryer (Büchi two fluid nozzle,
internal setting, 0.7 mm mixing needle and cap, 100% aspirator setting)

| | | | Total solid | | |
|---|---|---|---|---|---|
| | | | conc'n | Solvent | |
| Atomisation | Inlet | Outlet | (% w/w) | (host | Feed rate |
| pressure | temp | temp | in solvent | liquid) | (ml/min) |
| 5-6 bar | 150° C. | ˜100° C. | 1 | Aqueous | 5 |

There are a number of problems associated with the spray drying of pharmaceutically active agents. Firstly, there is the problem that the conventional spray drying processes and apparatus have a relatively low output for very fine powders and therefore are not particularly well suited to large scale production of pharmaceuticals. Secondly, most spray drying involves exposing the spray dried material to high temperatures, in order to ensure that the necessary evaporation takes place so that the dry particles are formed. Some temperature-sensitive active agents can be adversely affected by exposure to the temperatures used in conventional spray drying methods. A further disadvantage associated with conventional spray drying techniques is that the particles produced can have a broad range of particle sizes. This means that whilst some of the particles produced have the desired particle size, a proportion of the particles will not. Furthermore, this often results in a considerable quantity of the material, by mass, being larger than the desired particles size for delivery to the required site in the lung

Despite the foregoing, spray drying pharmaceutically active agents is still an accepted method of producing particles which are of a size suitable for administration by dry powder inhalation to the lungs.

Whilst spray drying can produce particles of a small enough size to be inhaled into the deep lung, these particles will frequently suffer from the agglomeration problems discussed above. Therefore, it will be necessary to modify the dry powder particles, in order to achieve good dispersion required for accurate dosing.

This modification may involve the simple addition of a force control agent to the spray dried particles of active material, as discussed above. Alternatively, the force control agent may be spray dried together with the active agent.

The co-spray drying of an active material and a force control agent has been disclosed in the prior art, albeit without proper recognition that the additives in question act as force control agents. For example, in WO 96/32149 (Inhale Therapeutic Systems), the co-spray drying of a pharmaceutically active agent and a carrier is proposed. The carrier is said to act as a bulking agent and may be, for example, a carbohydrate or an amino acid. There is little discussion of the spray drying technique, aside from that it involves the spray drying of an aqueous solution and conventional spray drying apparatus. Whilst it is suggested that the carrier may assist dispersal of the resultant spray dried particles, its inclusion does not seek to optimise this effect. The carrier is included in varying amounts and it would appear that this material is evenly distributed throughout the particles, with only a small proportion, if any, of the carrier material being present on the surfaces of the particles.

The inventors have now discovered that co-spray drying an active agent with a force control agent under specific conditions can result in particles with excellent properties which perform extremely well when administered by a DPI for inhalation into the lung.

In particular, it has been found that manipulating or adjusting the spray drying process can result in the force control agent being largely present on the surface of the particles. This clearly means that the force control agent will be able to reduce the tendency of the particles to agglomerate.

This allows dry powder compositions to be prepared which comprise co-spray dried active particles that exhibit a fine particle fraction (<5 μm) of at least 50%. Preferably, the FPF(ED) will be between 70 and 99%, more preferably between 80 and 99%. Furthermore, the FPF(MD) may be at least 50%. Preferably, the FPD will be between 50 and 99%, more preferably between 60 and 99%.

The effects of co-spray drying an active agent and a FCA are illustrated in the following discussion of various experiments and the results obtained. The experiments look at various variable factors in the spray drying process and investigate their effects on the nature and performance of the resultant particles.

In the experiments, the active agent used is heparin. The reason for selecting this active agent to illustrate and test the present invention is that heparin is a “sticky” compound and this tends to have a detrimental effect on the FPF and FPD of the dry powder. Therefore, obtaining good values of FPF and FPD using hepatin is an indication that the compositions really do exhibit good and improved properties, regardless of the “difficult” nature of the active agent included.

Unless otherwise indicated, the FPF(ED) and FPF(MD) figures given in the following sections of this specification were obtained by firing capsules, filled with approximately 20 mg of material, from a Monohaler into a multi stage liquid impinger (MSLI), at a flow rate of 901 pm, or a twin stage or rapid twin stage impinger (TSI or rTSI) at 601 pm. The “delivered dose” or “DD”, which is referred to in some of the following sections, is the same as the emitted dose or ED (as defined above).

In order to illustrate how the various variable factors of the spray drying process affects the properties of the resultant spray dried particles, firstly the effect of adjusting the solid concentration of active agent was investigated. The active agent was spray dried (without an FCA) using the standard parameters as shown in Table 11, but the solid concentration of active agent was increased from 10% w/w to 2 and 5% w/w total solids. The effects of these changes on the FPFs were then investigated and the results were as follows.

TABLE 12
FPF (%) less than 5 μm of the delivered dose (DD)
for spray dried heparin using “standard” spray drying parameters

| Description | Test | FPF < 5 μm (DD) (%) |
|---|---|---|
| 1% w/w heparin | MSLI | 17.0 |
| 1% w/w heparin | TSI | 20.3 |

The FPF for heparin spray dried alone, that is, without a co-spray dried FCA, using the “standard” spray drying parameters (see Table 11) was 17-20% as shown in Table 12. Testing was done with both a multi stage liquid impinger (MSLI) and a twin stage impinger (TSI).

TABLE 13
FPF (%) less than 5 μm of DD for heparin spray dried
from increasing solid concentrations

| Description | Test | FPF < 5 μm (DD) (%) |
|---|---|---|
| 2% w/w heparin | rTSI | 21.3 |
| 5% w/w heparin | rTSI | 8.3 |

Increasing the solid concentration of heparin from 1% w/w (Table 12) to 5% w/w (Table 13) caused a large reduction in FPF of heparin from approximately 20% FPF to 8.3%, when tested using a rapid-TSI. 2% w/w solid content did not seem to have an effect on FPF.

Thus, increasing the solid content of the feed solution did not improve the FPF of the active particles. Increasing the solid content as high as 5% w/w reduced the FPF by more than 10%. Increasing the solid content of a feedstock without changing any of the other parameters generally causes an increase in particle size, as each droplet will have a greater mass of solid which results in a larger particles upon drying.

Accordingly, although a solid content of up to 10% w/w active agent, and in some cases as much as 25% w/w active agent, can be used, it is preferred for up to 5% w/w, and more preferably up to 2% w/w active agent to be used in the spray drying process of the present invention. It is also preferred for at least 0.05% w/w, and more preferably for at least 0.5% w/w to be employed for practical purposes of production rate.

A further variable factor in the spray drying process is the nature of the feedstock, which may be a solution or a suspension and which can comprise a variety of different solvents or combinations thereof.

In some embodiments, all or at least a proportion of the active agent and/or FCA is or are in solution in the host liquid before being subjected to spray drying. Substantially all of the active agent and FCA can be in solution in the host liquid before being subjected to spray drying.

The active agent is preferably at least 1.5, 2, 4 and, more preferably, at least 10 times more soluble than the FCA in the host liquid at the spraying temperature and pressure. In preferred embodiments, this relationship exists at a temperature between 30 and 60° C. and atmospheric pressure. In other embodiments, this relationship exists at a temperature between 20 to 30° C. and atmospheric pressure, or, preferably, at 20° C. and atmospheric pressure.

The FCA may include one or more water soluble substances. This helps absorption of the substance by the body if the FCA reaches the lower lung. The FCA may include dipolar ions, which may be zwitterions.

Alternatively, the FCA may comprise a substance which is not soluble in water or which is only poorly soluble in water. Where such an FCA is used, it may be advantageous to include further agents to the mixture to be spray dried which will assist solubilising the FCA. For example, the FCA used could be magnesium stearate, which is only slightly soluble in water. However, the addition of an acid will help to solubilise the magnesium stearate and, as the acid will evaporate during the spray drying process, the resultant particles will not suffer from any “contamination” from the acid. Nevertheless, the use of a water soluble FCA is preferred, as the spray drying system is simpler and probably more predictable.

The host liquid preferably includes water. The liquid can employ water alone as a solvent or it may also include an organic co-solvent, or a plurality of organic co-solvents. A combination of water and one or more organic co-solvents is especially useful with active agents and FCAs that are insoluble or substantially insoluble in water alone. Preferred organic co-solvents include methanol, ethanol, propan-1-ol, propan1-2-ol and acetone, with ethanol being the most preferred.

In one embodiment of the present invention, the host liquid consists substantially of water. The use of this host liquid reduces any environmental cost or toxicological complications, or explosive risk. Hence, a host liquid consisting essentially of water provides a significant practical advantage and reduces the process costs.

If an organic solvent is present in the host liquid, it should be selected so that it produces a vapour which is significantly below any explosive or combustion limit. Also, preferably, the spraying composition does not include any blowing agent, such as ammonium carbonate or a halogenated liquid.

The effect of spray drying an active agent with various organic solvents was evaluated. The “standard” parameters as outlined in Table 11 were used to spray dry heparin, with the only difference being that the heparin was spray dried from 10% w/w organic solvent (propan-1-ol, methanol or ethanol) in water. The results are set out in Table 14.

TABLE 14
FPF (%) less than 5 μm of DD for heparin spray
dried from an organic solvent.

| Spray drying | | | |
|---|---|---|---|
| feedstock % w/w | | | FPF < 5 μm |
| heparin | Solvent % w/w | Test | (DD) (%) |
| 1 | 10% methanol | MSLI | 2.3 |
| 1 | 10% ethanol | MSLI | 6.2 |
| 1 | 10% propan-1-ol | MSLI | 2.0 |

Spray drying 1% w/w heparin from 10% methanol, ethanol and propan-1-ol resulted in a lowering of FPF (Table 14) from approximately 20% when spray dried from aqueous solvent using identical parameters (shown in Table 12) to 2-6% FPF.

One might expect that adding an organic solvent to the feedstock would cause an increase of the FPF, as a result of a reduction in the viscosity of the feedstock, and a lower energy input being required to generate smaller particles. However, the results obtained from 2-fluid nozzle spray drying of heparin from feedstocks containing 10% organic solvent (Table 14) show a reduction in FPF.

The reason for this change in the FPF may be due to the effect that the solvent has on the positioning of the important hydrophobic moieties of the drug or FCA whilst in the spray drying solution or suspension. The hydrophobic moieties are thought to have the significant force controlling effect. The exposure of a hydrophobic surface is believed to minimise any potential polar forces increasing surface adhesion, such as hydrogen bonds or permanent dipole effects, leaving only the ubiquitous weak London forces. The presence of these hydrophobic moieties on the surface of the particles is therefore important if the cohesion of the powder particles is to be limited, to provide better FPF performance.

When the FCA is in an aqueous solvent, the hydrophobic moieties will be repelled from the interior of the droplet, as the thermodynamics of the system will tend to drive a minimum interaction of these groups with the polar aqueous phase. The positioning of these moieties is therefore dictated by the nature of the solvent and this, in turn, affects the positioning of these groups in the eventual spray dried particles. When the aqueous solution of active agent and FCA is spray dried, it may be that the hydrophobic moieties are more likely to be positioned on the surfaces of the particles than if the active agent and FCA are dissolved in an organic solvent, such as ethanol or methanol.

As a further test of the parameters which might affect the nature of the spray dried particles, an active agent was spray dried using the standard parameters used above (Table 11), but the effect of temperature on the particles produced was investigated by spray drying with inlet temperatures of 75° C. to 220° C. The results are set out in Table 15.

TABLE 15
FPF (%) less than 5 μm of DD for heparin spray
dried using different inlet temperatures.

| Inlet | Approx. outlet | | |
|---|---|---|---|
| temperature | temperature | Test | FPF < 5 μm (DD) (%) |
| 220° C. | 135° C. | MSLI | 17.5 |
| 75° C. | 35° C. | rTSI | 22.5 |

Thus, it can be seen that spray drying heparin at a higher or lower inlet temperature relative to the “standard” 150° C. normally used did not offer a substantial improvement in FPF.

A preferable range for the inlet temperature is 40° C. to 300° C., preferably 75° C. to 220° C. A preferable range for the outlet temperature is 20° C. to 200° C., preferably 35° C. to 135° C.

The effects of co-spray drying an active agent with varying amounts of the 1-leucine, a FCA, from aqueous solution were then studied. Standard Büchi spray drying parameters were used, as shown in Table 11. L-leucine was included in the solution of heparin such that the percentage of 1-leucine ranged from 2-50% w/w. The results are set out in Table 16. 1% total solids solution was sprayed from a 2-fluid nozzle into a Büchi spray drier.

Blends of heparin and 1-leucine were prepared at different weight percentages of 1-leucine. Powders of 2%, 5%, 10%, 25% and 50% w/w 1-leucine were prepared. The spray drier feed flow rate was 120 ml/hr, the inlet temperature was 150° C., and flush nozzle setting was used. The schematic set-up of the two-fluid nozzle spray drier is shown in FIG. 39.

TABLE 16
FPF (%) less than 5 μm of DD for heparin co-spray dried with l-leucine.

| Spray drying | Co-spray drying | | |
|---|---|---|---|
| feedstock % w/w | with l-leucine % | | FPF < 5 μm |
| heparin | w/w | Test | (DD) (%) |
| 1 | 2% | rTSI | 20.0 |
| 1 | 5% | MSLI | 32.8 |
| 1 | 10% | MSLI | 30.8 |
| 1 | 25% | MSLI | 35.4 |
| 1 | 50% | MSLI | 51.7 |

The results show that increasing the percentage of 1-leucine included in the feedstock for spray drying resulted in a steady improvement in FPF from approximately 20% FPF with 2% leucine, to 50% FPF with 50% leucine (Table 16).

A further MSLI study was conducted using a feed rate of 300 ml/hr.

20 mg of powder was dispersed in each case and the results set out in Table 17 indicate an improvement of FPF with addition of a FCA, although the FPD does not improve with the addition of more than 10% 1-leucine due to the relative reduction of the heparin content.

TABLE 17
MSLI study of co-spray dried heparin
and varying concentrations of leucine

| | | ED | FPF % | FPD |
|---|---|---|---|---|
| Formulation | Test | (mg) | (emitted dose) | (mg) |
| Heparin (0% leucine) | MSLI | 10 | 17 | 1.8 |
| Heparin + leucine (5% w/w) | MSLI | 11 | 33 | 3.6 |
| Heparin + leucine (10% w/w) | MSLI | 13 | 31 | 3.9 |
| Heparin + leucine (25% w/w) | MSLI | 10 | 35 | 3.7 |
| Heparin + leucine (50% w/w) | MSLI | 6 | 52 | 3.0 |

Thus, the increased FPF is achieved even at low amounts of FCA. Whilst the active agent may be spray dried with from 0.1 to 50% w/w FCA to active agent, smaller amounts of FCA are preferred, in order to reduce the risk of toxicity problems. Preferably, the amount of FCA is no more than 10% w/w, more preferably, it is no more than 5% w/w, no more than 3% w/w, no more than 2% w/w or no more than 1% w/w.

In some embodiments, the FCA is an amino acid, and preferably the FCA is hydrophobic amino acid. One or more of the following amino acids may be used: leucine, preferably 1-leucine, isoleucine, lysine and cysteine. Most preferably, the active agent is co-spray dried with 1-leucine.

It has been found that co-spray drying an active agent with an FCA, and in particular with 1-leucine, isoleucine, lysine and cysteine, leads to significant changes in the particle cohesion, greatly enhancing the properties of the dry powder when administered by pulmonary inhalation.

Where the spray drying takes place under “standard” parameters and using conventional spray drying apparatus, it has been found that spray drying an active agent with an FCA can lead to non-spherical particle morphology. At low concentrations of FCA, the surfaces of the particles show dimples or depressions. As the amount of co-spray dried PCA is increased, these dimples become more extreme, with the particles eventually having a shrivelled or wrinkled surface.

The morphology of the particles was viewed using scanning electron micrographs (SEMs).

SEM micrographs of 2-fluid nozzle spray dried powders (FIGS. 40A-D) illustrate a clear relationship between the increasing percentage of 1-leucine and an increasingly dimpled or wrinkled surface of the particles. The particles with the highest 1-leucine content appear to be extremely wrinkled and, in selected cases, may even burst as an extreme result of “blowing”, a phenomenon whereby the particles form a shell or skin which inflates due to the evaporation of the solvent, creating a raised internal vapour pressure and then may collapse or burst.

Droplets from the two fluid nozzle are initially dried at a relatively high rate during spray drying. This creates a viscous layer of material around the exterior of the liquid droplet. As the drying continues, the viscous layer is firstly stretched (like a balloon) by the increased vapour pressure inside the viscous layer as the solvent evaporates. The solvent vapour diffuses through the growing viscous layer until it is exhausted and the viscous layer then collapses, resulting in the formation of craters in the surface or wrinkling of the particles.

FIG. 40A is an SEM micrograph of 2-fluid nozzle spray dried heparin. The particles are generally spherical in shape and the surfaces are substantially smooth. However, the particles each have one (smooth) crater or dimple in their surface.

FIG. 40B is an SEM micrograph of 2-fluid nozzle spray dried heparin with 5% leucine. The particles now exhibit more dimples or craters on their surface. The particles still have a generally smooth surface.

FIG. 40C is an SEM micrograph of 2-fluid nozzle spray dried heparin with 25% leucine. With the increase in FCA, the surface of the particles no longer appears smooth and the generally spherical shape has disappeared. The particles have a shrivelled, wrinkled appearance.

FIG. 40D is an SEM micrograph of 2-fluid nozzle spray dried heparin with 50% leucine. The shrivelling observed in the particles of FIG. 40C has become more pronounced and the particles appear to have inflated and then collapsed, looking like extremely wrinkled particles.

The net effect of the inflation, stretching of the skin and deflation is the creation of significant numbers of craters and wrinkles or folds on the particle surface, which consequently results in a relatively low density particle which occupies a greater volume than a smooth-surfaced particle.

This change in the surface morphology of these co-spray dried particles may contribute to reduced cohesion between the particles. Particles of pure active material are generally spherical in shape, as seen in FIG. 40A. It has been argued that increased particle surface roughness or rugosity, such as is caused by surface wrinkles or craters, results in reduced particle cohesion and adhesion by minimising the surface contact area between particles.

However, it has surprisingly been found that it is advantageuos not to produce severely dimpled or wrinkled particles, as these can yield low density powders, with very high voidage between particles. Such powders occupy a large volume relative to their mass as a consequence of this form, and can result in packaging problems, i.e., much larger blisters or capsules are required for a given mass of powder.

Advantageously, powders according to the present invention have a tapped density of more than 0.1 g/cc, mote than 0.2 g/cc, more than 0.3 g/cc, more than 0.4 g/cc, or more than 0.5 g/cc.

It has also been speculated that this particle morphology may even help the particles to fly when they are expelled for the inhaler device. This means that more of the active particles are capable of reaching the lower respiratory tract or deep lung. Despite this speculation relating to the benefits of the irregular shapes of the particles to be inhaled, the inventors actually feel that the chemical nature of the particle surfaces may be even more influential on the performance of the particles in terms of FPF, ED, etc. In particular, it is thought that the presence of hydrophobic moieties on the surface of particles is thought to be more significant in reducing cohesion that the presence of craters or dimples. As mentioned above, it is believed that the solvent used in the feedstock may be able to influence the chemical properties of the particle surfaces. Therefore, contrary to the suggestion in the prior art, it is not necessary to seek to produce extremely dimpled or wrinkled particles in order to provide good FPF values.

Next, the effect of spray drying an active agent with various excipients was investigated. Standard spray drying parameters as shown in Table 11 were used and the various excipients tested were lactose, dextrose, mannitol and human serum albumin (HSA). The excipients were co-spray dried with heparin from aqueous solution. Between 5-50% w/w of the excipients were included, with total solid content not exceeding 1% w/w of the solution.

TABLE 18
FPF (%) less than 5 μm of DD for heparin co-spray dried with excipients.

| Spray drying | Co-spray drying | | FPF < 5 μm (DD) |
|---|---|---|---|
| feedstock % w/w | excipient % w/w | Test | (%) |
| 1 | 5% lactose | rTSI | 7.0 |
| 1 | 20% lactose | rTSI | 5.3 |
| 1 | 50% lactose | rTSI | 10.3 |
| 1 | 5% dextrose | rTSI | 11.0 |
| 1 | 50% dextrose | rTSI | 1.7 |
| 1 | 5% mannitol | rTSI | 14.0 |
| 1 | 20% mannitol | rTSI | 11.3 |
| 1 | 5% HSA | rTSI | 34.0 |
| 1 | 50% HSA | rTSI | 28.0 |

Inclusion of lactose (5-50%), dextrose (5-50%) and mannitol (5-20%) did not improve the FPF (Table 18). In fact, for all of these excipients, FPFs fell to below the “standard” 20% for spray dried heparin. However, inclusion of 5% HSA gave an improvement.

As the presence of the HSA in the active particle clearly reduces the particle cohesion, thereby increasing the FPF, HSA may be considered, for the purpose of the present invention, to be a FCA. However, in some embodiments of the invention, the FCA used is preferably not HSA.

It is believed that the ability of HSA to act as an FCA when co-spray dried as described above may be due to the arrangement of the hydrophobic moieties of the HSA on the surface of the spray dried particles. As discussed above, the positioning of hydrophobic groups on the surface of the spray dried particles is considered to be very important and can affect the cohesiveness and adhesiveness of the particles in a dry powder formulation. Proteins, such as HSA, tend to have hydrophobic parts of their constituent amino acids which allow them to act as FCAs under the appropriate conditions. Indeed, in one embodiment of the present invention, where the active agent is a protein, under the correct spray drying conditions, the active agent may itself act as an FCA, thereby avoiding the need to spray dry the protein with a separate FCA. The protein must be spray dried in a manner that will allow the hydrophobic moieties to be arranged on the surface of the resultant particles. Therefore, the host solution is preferably an aqueous solution. Additionally, the drying of the particles should occur at a rate which allows the movement of the hydrophobic moieties or retention of the moieties at the surface.

Thus, according to one aspect of the present invention, a method is provided for producing spray dried particles comprising a protein as both the active agent and an FCA. The particles exhibit FPF(ED) and FPF(MD) which is better than those exhibited by conventionally spray dried particles of protein, as a result of the hydrophobic moieties arranged on the surface of the spray dried particles according to the present invention.

Alternative Droplet Formation

It has further been discovered that the FPF and FPD of the dry powder formulation is also affected by the means used to create the droplets which are spray dried. Different means of forming droplets can affect the size and size distribution of the droplets, as well as the velocity at which the droplets travel when formed and the gas flow around the droplets. In this regard, the velocity at which the droplets travel when formed and the gas (which is usually air) flow around the droplets can dramatically affect size, size distribution and shape of resulting dried particles.

This aspect of the spray drying process is therefore important in the inventors' attempts to engineer particles with chemical and physical properties that provide good performance which the particles are administered via pulmonary inhalation.

It has been found that the formation of the droplets in the spray drying process may be controlled, so that droplets of a given size and of a narrow size distribution may be formed. Furthermore, controlling the formation of the droplets can allow control of the air flow around the droplets which, in turn, can be used to control the drying of the droplets and, in particular, the rate of drying. Controlling the formation of the droplets may be achieved by using alternatives to the conventional 2-fluid nozzles, especially avoiding the use of high velocity air flows. The following discussion of the use of alternative droplet forming means can be used in combination with all of the foregoing factors which provide improvements in the performance of the spray dried particles, as will become clear.

According to another aspect of the present invention, a method of preparing a dry powder composition is provided, wherein the active agent is spray dried using a spray drier comprising a means for producing droplets moving at a controlled velocity and of a predetermined droplet size. The velocity of the droplets is preferably controlled relative to the body of gas into which they are sprayed. This can be achieved by controlling the droplets' initial velocity and/or the velocity of the body of gas into which they are sprayed.

It is clearly desirable to be able to control the size of the droplet formed during the spray drying process and the droplet size will affect the size of the dried particle. Preferably, the droplet forming means also produces a relatively narrow droplet, and therefore particle, size distribution. This will lead to a dry powder formulation with a more uniform particle size and thus a more predictable and consistent FPF and FPD, by reducing the mass of particles with a size above a defined limit, preferably 90% below 5 μm, below 3 μm or below 2 μm.

The ability to control the velocity of the droplet also allows further control over the properties of the resulting particles. In particular, the gas speed around the droplet will affect the speed with which the droplet dries. In the case of droplets which are moving quickly, such as those formed using a 2-fluid nozzle arrangement (spraying into air), the air around the droplet is constantly being replaced. As the solvent evaporates from the droplet, the moisture enters the air around the droplet. If this moist air is constantly replaced by fresh, dry air, the rate of evaporation will be increased. In contrast, if the droplet is moving through the air slowly, the air around the droplet will not be replaced and the high humidity around the droplet will slow the rate of drying. As discussed below in greater detail, the rate at which a droplet dries affects various properties of the particles formed, including FPF and FPD.

Preferably, the velocity of droplets at 10 mm from their point of generation is less than 100 m/s, more preferably less than 50 m/s, most preferably less than 20 m/s. Preferably the velocity of the gas, used in the generation of the droplets, at 10 mm from the point at which they are generated is less than 100 m/s, more preferably less than 50 m/s, most preferably less than 20 m/s. In an embodiment, the velocity of the droplets relative to the body of gas into which they are sprayed, at 10 mm from their point of generation, is less than 100 m/s, more preferably less than 50 m/s, most preferably less than 20 m/s.

Preferably, the velocity of droplets at 5 mm from their point of generation is less than 100 m/s, more preferably less than 50 m/s, most preferably less than 20 m/s. Preferably the velocity of the gas, used in the generation of the droplets, at 10 mm from the point at which they are generated is less than 100 m/s, more preferably less than 50 m/s, most preferably less than 20 m/s. In an embodiment, the velocity of the droplets relative to the body of gas into which they are sprayed, at 10 mm from their point of generation, is less than 100 m/s, more preferably less than 50 m/s, most preferably less than 20 m/s.

Preferably, the output per single piezo unit (for such a unit oscillating at >1.5 MegaHz) is greater than 1.0 cc/min, greater than 3.0 cc/min, greater than 5.0 cc/min, greater than 8.0 cc/min, greater than 10.0 cc/min or greater than 15.0 cc/min. Such units should then produce dry particles with D(90) as measured by Malvern Mastersizer from a dry powder dispersion unit of less than 3 μm, less than 2.5 μm or less than 2 m/s.

Preferably, the output per single piezo unit (for such a unit oscillating at >2.2 MegaHz) is greater than 0.5 cc/min, greater than 1.0 cc/min, greater than 3.0 cc/min, greater than 5.0 cc/min, greater than 8.0 cc/min, greater than 10.0 cc/min or greater than 15.0 cc/min. Such units should then produce dry particles with D(90) as measured by Malvern Mastersizer from a dry powder dispersion unit of less than 3 μm, less than 2.5 μm, or less than 2 μm.

Preferably, the means for producing droplets moving at a controlled velocity and of a predetermined size is an alternative to the commonly used 2-fluid nozzle. In one embodiment, an ultrasonic nebuliser (USN) is used to form the droplets in the spray drying process.

Whilst ultrasonic nebulisers (USNs) are known, these are conventionally used in inhaler devices, for the direct inhalation of solutions containing drug, and they have not previously been widely used in a spray drying apparatus. It has been discovered that the use of such a nebuliser in spray drying has a number of important advantages and these have not previously been recognised. The preferred USNs control the velocity of the particles and therefore the rate at which the particles are dried, which in turn affects the shape and density of the resultant particles. The use of USNs also provides an opportunity to perform spray drying on a larger scale than is possible using conventional spray drying apparatus with conventional types of nozzles used to create the droplets, such as 2-fluid nozzles.

USNs use an ultrasonic transducer which is submerged in a liquid. The ultrasonic transducer (a piezoelectric crystal) vibrates at ultrasonic frequencies to produce the short wavelengths required for liquid atomisation. In one common form of USN, the base of the crystal is held such that the vibrations are transmitted from its surface to the nebuliser liquid, either directly or via a coupling liquid, which is usually water. When the ultrasonic vibrations are sufficiently intense, a fountain of liquid is formed at the surface of the liquid in the nebuliser chamber. Large droplets are emitted from the apex and a “fog” of small droplets is emitted. A schematic diagram showing how a standard USN works is shown in FIG. 41.

The attractive characteristics of USNs for producing fine particle dry powders include: low spray velocity; the small amount of carrier gas required to operate the nebulisers; the comparatively small droplet size and narrow droplet size distribution produced; the simple nature of the USNs (the absence of moving parts which can wear, contamination, etc.); the ability to accurately control the gas flow around the droplets, thereby controlling the rate of drying; and the high output rate which makes the production of dry powders using USNs commercially viable in a way that is difficult and expensive when using a conventional two-fluid nozzle arrangement. This is because scaling up of conventional spray drying apparatus is difficult and the use of space is inefficient in conventional spray drying apparatus which means that large scale spray drying requires many apparatus and much floor space.

USNs do not separate the liquid into droplets by increasing the velocity of the liquid. Rather, the necessary energy is provided by the vibration caused by the ultrasonic nebuliser.

Furthermore, the USNs may be used to adjust the drying of the droplets and to control the expression of the force control agent on the surface of the resultant particles. Where the active agent itself can act as a force control agent, spray drying with a USN can further help to control the positioning of the hydrophobic moieties so that the effect of including a force control agent can be achieved even without including one.

Thus, as an alternative to the conventional Büchi two-fluid nozzle, an ultrasonic nebuliser may be used to generate droplets of active agent, which are then dried within the Büchi drying chamber. In one arrangement, the USN is placed in the feed solution comprising an active agent in a specially designed glass chamber which allows introduction of the cloud of droplets generated by the USN directly into the heated drying chamber of the spray dryer.

The two-fluid nozzle is left in place to seal the hole in which it normally sits, but the compressed air was not turned on. The drying chamber is then heated up to 150° C. inlet temperature, with 100% aspirator setting. Due to the negative pressure of the Büchi system, the nebulised cloud of droplets is easily drawn into the drying chamber, where the droplets are dried to form particles, which are subsequently classified by the cyclone, and collected in the collection jar. It is important that the level of feed solution in the chamber is regularly topped up to avoid over concentration of the feed solution as a result of continuous nebulisation.

Two theories have been developed which describe the mechanism of liquid disintegration and aerosol production in ultrasonic devices (Mercer 1981; 1968 and Sollner 1936). Lang (1962) observed that the mean droplet size generated from thin liquid layers was proportional to the capillary wavelength on the liquid surface. Using the experimentally determined factor of 0.34, the droplet diameter D is given by:
dₚ=0.34 (8πy/pf²)^{1/3}
p=solution density g cm⁻³ (water=1)
y=surface tension dyn cm⁻¹ (water=70)
f=frequency (MHz)

This means that for a frequency of 1.7 MHz the calculated droplet size is 2.9 μm and for 2.4 MHz the calculated droplet size is 2.3 μm. Atomisers are also available with frequencies up to 4 MHz with a calculated droplet size of 1.6 μm.

Clearly, this allows the size of the droplets to be accurately and easily controlled, which in turn means that the active particle size can also be controlled (as the dried particle size will depend, to a great extent, on the size of the droplet). Further, the USN provides droplets which are smaller than can be practically produced at a comparative output by a conventional 2 fluid nozzle.

In an embodiment of the present invention, the method of preparing the active particles involves the use of an ultrasonic nebuliser. Preferably, the ultrasonic nebuliset is incorporated in a spray drier.

One type of ultrasonic nebuliser which may be used in the present invention is described in the European Patent Application No. 0931595A1. This patent application described ultrasonic nebulisers which work extremely well in putting the present invention into practice.

Despite the fact that the ultrasonic nebulisers disclosed in this document are not envisaged as being part of a spray drying apparatus, the nebulisers may be simply and easily incorporated into a spray drier to produce excellent spray dried particles as indicated above;

The nebulisers disclosed in EP 0931595 A1 are used as air humidifiers. However, the droplets produced are of an ideal size range with a small size distribution for use in a spray drying process. What is more, the nebulisers have a very high output rate of several litres of feed liquid per hour and up to of the order of 60 litres per hour in some of the devices produced and sold by the company Areco. This is very high compared to the 2-fluid nozzles used in conventional spray drying apparatus and it allows the spray drying process to be carried out on a commercially viable scale.

Other suitable ultrasonic nebulisers are disclosed in U.S. Pat. No. 6,051,257 and in WO 01/49263.

A further advantage of the use of USNs to produce droplets in the spray drying process is that the particles which are produced are small, spherical in shape and are dense. These properties provide improved dosing. Furthermore, it is thought that the size and shape of the particles produced reduces the drug's device retention to very low levels.

In addition, the USNs can produce very small droplets relative to other known atomiser types and this, in turn, leads to the production of very small particles. The particles produced by USNs tend to be within the size range of 0.5 to 5 μm, or even 0.5 to 3 μm. This compares very favourably with the particle sizes which tend to be obtained using conventional spray drying techniques and apparatus, or obtained by milling. Both of these latter methods produce particles with a minimum size of around 1 μm. These advantages associated with the use of USNs are discussed in greater detail below.

A USN was used to prepare dry powders using a feed solution of an active agent (heparin) alone, and a blend of active agent with 1% to 5% and 10% w/w FCA (1-leucine). The ultrasonic nebuliser output rate was 130 ml/hr. The furnace temperature of the nebulised powders was set at 350° C. FIG. 42 shows a schematic drawing of the ultrasonic set-up.

In order to test the processing of the powders, work was conducted using a Monohaler and a capsule filled with 20 mg powder and fired into a rapid TSI in the manner explained previously. The study used a TSI flow rate of 601 pm with a cut-off of approximately 5 μm.

Three measurements were made for each blend and the results are summarised below in Table 19, giving the average values of the three sets of results obtained.

TABLE 19
rapid TSI results using the dry powder produced using a
USN with varying amounts of FCA

| Formulation | FPF % (metered dose) | FPD (mg) |
|---|---|---|
| Heparin (0% leucine) | 1.1 | 0.22 |
| Heparin + leucine (1% w/w) | 17.4 | 3.5 |
| Heparin + leucine (2% w/w) | 30.2 | 6.0 |
| Heparin + leucine (3% w/w) | 28.6 | 5.7 |
| Heparin + leucine (4% w/w) | 48.4 | 9.7 |
| Heparin + leucine (5% w/w) | 41.5 | 8.3 |
| Heparin + leucine (10% w/w) | 55.8 | 11.8 |

The rapid TSI results using the dry powder produced using the USN indicate a very low aerosolisation efficiency for pure heparin particles, but an improvement appeared in FPF with addition of l-leucine as a FCA.

The reason for the poor performance of the pure drug particles compared to those produced using the two-fluid nozzle arrangement is due to the size of the particles produced by these two different processes. The particles of pure drug generated using the USNs are extremely small (D(50) in the order of 1 μm) compared to those prepared using the two-fluid nozzle arrangement D(50) in the order of 2.5%). Without a FCA, the smaller particles produced using the USN exhibit a worse FPF than the larger particles produced by the two-fluid nozzle.

The morphology of the particles was viewed using scanning electron micrographs (SEMs).

FIG. 43A shows SEM micrographs of USN spray dried heparin alone, whilst FIG. 43B shows SEM micrographs of USN spray dried heparin with 10% leucine.

As can be clearly seem from the SEMs, the shape of particles formed by co-spray drying an active agent and leucine using a USN differs to that of particles formed by co-spray drying heparin and leucine using a conventional 2-fluid nozzle spray drying technique.

The SEM micrographs of pure heparin generated using a USN show that the particles have a size of approximately 2 μm or less. The SEMs also show that these particles tend to form “hard” agglomerates of up to 200 μm.

In contrast, the SEMs of nebulised heparin and leucine show that the primary particles produced are of the same size as the pure heparin particles. However, these particles are discrete and agglomerates are less evident and less compacted in nature.

What is more, the distinctive dimples or wrinkles observed on the surface of the particles prepared by co-spray drying heparin and leucine using a 2-fluid nozzle spray drier (FIGS. 40A-40D) are less evident when the particles are spray dried using a USN. Despite this, the co-spray dried particles formed using a USN still have an improved FPF and FPD over particles formed in the same way but without the FCA. In this case, this improvement is clearly not primarily due to the shape of the particles, nor is it due to any increase in density or rugosity.

It is believed that the leucine concentration at the surface of the solid particles is governed by several factors. These include the concentration of leucine in the solution which forms the droplets, the relative solubility of leucine compared to the active agent, the surface activity of leucine, the mass transport rate within the drying droplet and the speed at which the droplets dry. If drying is very rapid it is thought that the leucine content at the particle's surface will be lower than that for a slower drying rate. The leucine surface concentration is determined by the rate of leucine transport to the surface, and its precipitation rate, during the drying process.

As mentioned above, high gas flow rates around the droplets can accelerate drying and it is thought that, because the gas speed around droplets formed using a USN is low in comparison to that around droplets formed using conventional 2-fluid nozzles, droplets formed using the former technique dry more slowly than those produced by using conventional 2-fluid nozzles. The leucine (or other FCA) concentration on the shell of droplets and dried particles produced using a USN can be higher as a result. It is considered that these effects reduce the rate of solvent evaporation from the droplets and reduce “blowing” and, therefore, are responsible for the physically smaller and smoother primary particles we have observed (Kodas, T. T and Hampden Smith, M., 1999, Aerosol Processing of materials, 440). In this last regard, and as previously noted, droplets formed by the 2-fluid nozzle system have rapid air flow around them and they, therefore, dry very rapidly, and markedly exhibit the effects of blowing.

It is also speculated that the slower drying rate which is expected when the droplets are formed using USNs allows the FCA to migrate to the surface of the droplet during the drying process. This migration may be further assisted by the presence of a solvent which encourages the hydrophobic moieties of the FCA to become positioned on the surface of the droplet. An aqueous solvent is thought to be of assistance in this regard.

With the FCA being able migrate to the surface of the droplet so that it is present on the surface of the resultant particle, it is clear that a greater proportion of the FCA which is included in the droplet will actually have the force controlling effect (as the FCA must be present on the surface in order for it to have this effect). Therefore, it also follows that the use of USNs has the further advantage that it requires the addition of less FCA to produce the same force controlling effect in the resultant particles, compared to particles produced using conventional spray drying methods.

Thus, it will not be necessary to include amounts of up to 50% w/w of FCA in the feed solution, as suggested in the prior art discussed above. Rather, it has been found that excellent FPF values are achieved when no more than 20% W/w FCA is included. Preferably, no more than 10% w/w, no more than 8% w/w, no more than 5% w/w, no more than 4% w/w, no more than 2% w/w or no more than 1% w/w FCA is spray dried using a USN. The amount of FCA included may be as low as 0.1% w/w where the active agent is not able to act as an FCA itself.

Naturally, where the active agent itself has hydrophobic moieties which can be presented as a dominant composition on the particle surface, no FCA need be included.

The movement of the FCA during the drying step of the spray drying process will also be affected by the nature of the solvent used in the host liquid. As discussed above, an aqueous solvent is thought to assist the migration of the hydrophobic moieties to the surface of the droplet and therefore the surface of the resultant particle, so that the force controlling properties of these moieties is maximised.

In a particle size study, the particle size of the spray dried particles formed using the USN was analysed. The dry powders were dispersed at 4 bar in a Malvern Mastersizer 2000, using a Scirocco dry powder unit. The values of D10, D50 and D90 of the ultrasonic nebulised powders were measured and are indicated in Table 21 (10% by volume of the particles are of a size, measured by Malvern, that is below the D10 value. 50% by volume of the particles are of a size, measured by Malvern, that is below the D50 value and so on). The values are an average of three measurements.

In addition, the percentage mass of particles with a size of less than 5 μm was obtained from the particle size data and is expressed as FPF.

TABLE 20
Particle size study of spray dried particles using USN,
without secondary drying

| | D10 | D50 | D90 | |
|---|---|---|---|---|
| Formulation | (μm) | (μm) | (μm) | FPF % (<5 μm) |
| Heparin (0% leucine) | 0.43 | 1.07 | 4.08 | 90.52 |
| Heparin + leucine (1% w/w) | 0.41 | 0.90 | 1.79 | 99.97 |
| Heparin + leucine (2% w/w) | 0.41 | 0.89 | 1.75 | 100 |
| Heparin + leucine (3% w/w) | 0.41 | 0.88 | 1.71 | 100 |
| Heparin + leucine (4% w/w) | 0.41 | 0.86 | 1.71 | 100 |
| Heparin + leucine (5% w/w) | 0.41 | 0.90 | 1.84 | 100 |
| Heparin + leucine (10% w/w) | 0.41 | 0.89 | 1.76 | 100 |

FIG. 44 shows a typical size distribution curve of three repeated tests of pure heparin powder generated using an ultrasonic nebuliser. The main peak represents the size of the individual active particles, ranging between 0.2 μm and 4.51 μm in diameter. The second, smaller peak between diameters of 17 to 35 μm represents agglomerates of active particles.

Sympatec particle sizing (Helos dry dispersed) results showed that ultrasonic nebulised powders have a narrower size distribution and smaller mean particle size than the 2-fluid nozzle spray dried powders.

FIG. 45A shows a comparison between particle size distribution curves of 2-fluid nozzle spray dried powders and ultrasonic nebulised powders comprising a blend of heparin with 2% leucine w/w.

FIG. 45B shows a comparison between particle size distribution curves of 2-fluid nozzle spray dried powders and ultrasonic nebulised powders comprising a blend of heparin with 5% leucine w/w.

FIG. 45C shows a comparison between particle size distribution curves of 2-fluid nozzle spray dried powders and ultrasonic nebulised powders comprising a blend of heparin with 10% leucine w/w.

These figures show a gradual disappearance of the second peak, indicating that the incidence of agglomerates is reduced as the amount of co-spray dried FCA is increased.

For the USN, spray dried material, agglomerate peaks disappears under the same test conditions when >3% leucine is added. For the 2-fluid nozzle spray dried material, agglomerate peaks disappear under the same test conditions when >10% leucine is added. This indicates that adding leucine as an FCA reduces the strength of the agglomerates in heparin powder. It further suggests that ultrasonic nebulised materials de-agglomerate more easily at lower leucine (FCA) contents. This may be related to the surface concentration of the leucine (FCA), as mentioned above.

The SEM images of ultrasonic nebulised powders (FIGS. 43A and 43B) also support the finding that addition of leucine facilitates aerosolisation. SEMs of pure heparin showed that although heparin primary particles are <2 μm, large distinct agglomerates are formed. The SEMs of all of the powders comprising heparin and leucine show that the primary particle size is still <2 μm, but the large agglomerates are not evident.

It can be seen that particles formed using a spray drying process involving an ultrasonic nebuliser have been found to have a greater FPF than those produced using a standard spray drying apparatus, for example with a two fluid nozzle configuration.

What is more, the particles formed using a spray drying process using a USN have been found to have a narrower particle size distribution than those produced using a standard spray drying apparatus, for example with a two fluid nozzle configuration.

Studies of the particles produced by spray drying using USNs have led to the discovery that the bulk density of ultra-fine drug powders can be beneficially increased whilst also improving aerosolisation characteristics. This finding is contrary to conventional thinking and in marked contrast to the prior art approaches to improving aerosolisation, whereby drug particles and formulations are prepared having reduced density. Whilst low density particles can improve aerosolisation, they place significant limitations on payload mass which can be delivered as a single inhalation. For example, a size 3 capsule (the type of capsule used in Cyclohaler™, Rotahaler™ and many other capsule-based DPIs) which conventionally holds 20 mg of formulated powder might only accommodate 5 mg or less of low density material.

The significance and commercial benefit of high density or densified powder particles is that it provides the potential to deliver increased powder payloads in smaller volumes. For example, a size 3 capsule which conventionally holds a 20 mg payload, may be able to accommodate up to 40 mg of a high density powder formulation and an Aspirair™ blister designed to hold a 5 mg payload may be used to hold 15 mg of a high density powder such as that which may be produced using the present invention. This is particularly important for drugs requiring high dose delivery, including, for example, heparin, where doses in the region of 40-50 mg may be required. It should be possible to incorporate this dose in the form of a high density powder into a blister or capsule which holds just 20-25 mg of a standard density powder.

Using the above described spray drying process using a USN, the final density of particles comprising active agent and FCA (heparin and leucine) has been increased by controlled atomisation and drying. The ability to increase density, as noted above, provides an opportunity to increase drug payloads filled into a unit blister or capsule whilst, in this case, raising FPD from 20% for conventionally spray dried heparin to 70% for heparin and an FCA spray dried according to the present invention.

The key to improved aerosolisation in a denser particle is the presence of FCA, without which the benefits of densification cannot be realised. The process by which densification is brought about is also critical in terms of the spatial positioning of the FCA on the drug particle surface. The aim is always to provide the maximum possible surface presence of FCA in the densified drug composite; In the case of the spray drying according to the present invention, conditions are selected to provide FCA surface enrichment of resultant drug particles.

Similar results to those shown above when using USNs are expected for spray drying using other means which produce low velocity droplets at high output rates. For example, further alternative nozzles may be used, such as electrospray nozzles or vibrating orifice nozzles. These nozzles, like the ultrasonic nozzles, are momentum free, resulting in a spray which can be easily directed by a carrier air stream, however, their output rate is generally lower.

Another attractive type of nozzle for use in a spray drying process is one which utilises electro-hydrodynamic atomisation. A tailor cone is created at a fine needle by applying high voltage at the tip. This shatters the droplets into an acceptable monodispersion. This method does not use a gas flow, except to transport the droplets after drying. An acceptable monodispersion can also be obtained utilising a spinning disc generator.

The nozzles such as ultrasonic nozzles, electrospray nozzles or vibrating orifice nozzles can be arranged in a multi nozzle array, in which many single nozzle orifices are arranged in a small area and facilitate a high total throughput of feed solution. The ultrasonic nozzle is an ultrasonic transducer (a piezoelectric crystal). If the ultrasonic transducer is located in an elongate vessel the output may be raised significantly.

Moisture Profiling

The spray drying process may include a further step wherein the moisture content of the spray dried particles is adjusted to allow fine-tuning of some of the properties of the particles.

When active particles are produced by spray drying, some moisture will remain in the particles. This is especially the case where the active agent is temperature sensitive and does not tolerate high temperatures for the extended period of time which would normally be required to remove further moisture from the particles.

The amount of moisture in the particles will affect various particle characteristics, such as density, porosity, flight characteristics, and the like.

Therefore, according to a further aspect of the present invention, a method of preparing a dry powder composition is provided, wherein the method comprises a step of adjusting the moisture content of the particles.

In one embodiment, the moisture adjustment or profiling step involves the removal of moisture. Such a secondary drying step preferably involves freeze-drying, wherein the additional moisture is removed by sublimation. An alternative type of drying for this purpose is vacuum drying.

Generally, the secondary drying takes place after the active has been co-spray dried with a force control agent. In another embodiment, the secondary drying takes place after nebulised active agent has been spray dried, wherein the active agent was optionally in a blend with a FCA.

The secondary drying step has two particular advantages. Firstly, it can be selected so as to avoid exposing the pharmaceutically active agent to high temperatures for prolonged periods. Furthermore, removal of the residual moisture by secondary drying is significantly cheaper than removing all of the moisture from the particle by spray drying. Thus, a combination of spray drying and freeze-drying or vacuum drying is economical and efficient, and is suitable for temperature sensitive pharmaceutically active agents.

In order to establish the effect of secondary drying of the powders, samples of active agent alone and of a combination of active agent (heparin) and an FCA (leucine 10% w/w), were secondary dried at 50° C. under vacuum for 24 hours.

The result set out in Table 21 indicate the secondary drying step further raised the FPF and FPD, when they are compared to the results in Table 20, which relates to equivalent particles which have not undergone secondary drying.

TABLE 21
rapid TSI results using the dry powder produced using a
USN with varying amounts of FCA, after secondary drying

| Formulation | FPF % (metered dose) | FPD (mg) |
|---|---|---|
| Heparin (0% leucine) | 4.1 | 0.82 |
| Heparin + leucine (10% w/w) | 70.8 | 14.2 |

In a later stage experiments have been conducted on samples of active agent (heparin) and an FCA (leucine 5% w/w), were secondary dried at 40° C. under vacuum for 24 hours.

Particle size tests were also conducted to show the effect of secondary drying. The particle size of the spray dried particles formed using the USN was analysed. The dry powders were dispersed at 4 bar in a Helos disperser. The powders were secondary dried over 24 hours under vacuum.

The values of FPF <5 μm and D10, D50 and D90 of the ultrasonic nebulised powders were measured and are indicated in Table 22.

TABLE 22
Particle size study of spray dried particles
using USN, after secondary drying

| Formulation | D10 | D50 | D90 | FPF % (<5 μm) |
|---|---|---|---|---|
| Heparin (0% leucine) | 0.44 | 1.06 | 2.93 | 92.35 |
| Heparin + leucine (10% w/w) | 0.40 | 0.87 | 1.77 | 100 |

Thus, by comparing the results in Table 22 with those of Table 20, one can see that secondary drying particles did not result in any significant change in particle size, both for active agent alone and for a blend of active agent and FCA.

FIG. 44 shows a comparison between particle size distribution curves of secondary dried and not secondary dried powders. The powder used was heparin with 10% leucine w/w. Clearly, there is virtually no difference between the curves, illustrating that secondary drying does not have an effect on particle size.

Then, in order to establish whether the effect of secondary drying varied between particles produced using a USN and a 2-fluid nozzle, the particle size study of secondary drying with spray dried particles formed using the USN was repeated but using a 2-fluid nozzle spray drier. Once again, the powders were secondary dried over 24 hours under vacuum. Values of FPF <5 μm and D10, D50 and D90 of the spray dried powders are indicated in Table 23 below.

TABLE 23
Particle size study of 2-fluid nozzle spray
dried particles after secondary drying

| Formulation | D10 | D50 | D90 | FPF % (<5 μm) |
|---|---|---|---|---|
| Heparin + leucine (2% w/w) | 0.59 | 2.09 | 5.19 | 89.57 |
| Heparin + leucine (5% w/w) | 0.61 | 2.16 | 4.77 | 91.18 |
| Heparin + leucine (10% w/w) | 0.58 | 2.04 | 3.93 | 96.6 |
| Heparin + leucine (25% w/w) | 0.63 | 2.34 | 4.85 | 91.15 |
| Heparin + leucine (50% w/w) | 1.05 | 3.03 | 6.62 | 80.03 |

FIGS. 40E to 40H show SEM micrographs of 2-fluid nozzle spray dried heparin with 2, 5, 10 and 50% leucine, after secondary drying. When one compares the particles in these Figures to those in FIGS. 40A to 40D, it can be seen that the secondary drying does appear to increase the “collapse” of the particles. Thus, even at low percentages of FCA, the secondary dried particles have a more wrinkled or shrivelled shape.

TABLE 24
Moisture content of 2 fluid nozzle spray dried
particles under standard condition

| | % w/w Moisture before | % w/w Moisture after |
|---|---|---|
| Formulation | secondary drying | secondary drying |
| Heparin + Leucine 5% | 9.57 | 2.18 |

The above discussed experiments and the moisture content values determined by Karl-Fisher methodology set out in Table 24 show that secondary drying significantly reduces the moisture content of heparin particles (by approximately 6.5%). This would imply that the heparin is drying in such a way that there is a hard outer shell holding residual moisture, which is driven off by secondary drying, and entrapped moisture is trapped with in a central core. One could infer that the residence time of the particle in the drying chamber is too short, and that the outer shell is being formed rapidly and is too hard to permit moisture to readily escape during the initial spray drying process.

Secondary drying can also be beneficial to the stability of the product, by bringing down the moisture content of a powder. It also means that drugs which may be very heat sensitive can be spray dried at lower temperatures to protect them, and then subjected to secondary drying to reduce the moisture further, and protect the drug. In another embodiment of the third aspect of the invention, the moisture profiling involves increasing the moisture content of the spray dried particles.

Preferably, the moisture is added by exposing the particles to a humid atmosphere. The amount of moisture added can be controlled by varying the humidity and/or the length of time for which the particles are exposed to this humidity.

Ultrasonic nebulised formulations comprising clomipramine and heparin were prepared next and were tested in Aspirair™ and MonoHaler™ devices.

The heparin formulation was produced from the original powder, using a spray drying system according to the present invention, as described above. This system comprises an ultrasonic nebulisation unit, a gas flow for transporting the droplets nebulised into a heated tube to dry the droplets, and a filtration unit for collecting the dried particles.

An aqueous solution of the heparin was made containing 1% w/w relative to the water. Leucine, a force control agent, was added to this in an amount sufficient to make 5% w/w relative to the heparin.

The solution was nebulised with a frequency of 2.4 MHz and guided through the tube furnace with furnace surface temperature heated to approximately 300° C., after which the dried powder was collected. The gas temperature was not measured, but was substantially less than this temperature. Malvern (dry powder) particle size measurement gave a d(50) of 0.8 μm.

The clomipramine hydrochloride formulation was produced from the original powder, using the same spray drying system as noted above for hepatin. This system comprises an ultrasonic nebulisation unit, a gas flow for transporting the droplets nebulised into a heated tube to dry the droplets, and a filtration unit for collecting the dried particles.

An aqueous solution of the clomipramine hydrochloride was made containing 2% w/w relative to the water. Sufficient leucine was added to make 5% w/w relative to the drug.

The solution was nebulised with a frequency of 2.4 MHz and guided through the tube furnace with furnace surface temperature heated to approximately 300° C., after which the dried powder was collected. The gas temperature was not measured, but was substantially less than this temperature. Malvern (dry powder) particle size measurement gave a d(50) of 1.1 μm.

The Malvern particle size distributions show that both the heparin and the clomipramine hydrochloride have very small particle sizes and distributions. The d(50) values are 0.8 μm for heparin and 1.1=μm for clomipramine hydrochloride. The modes of the distribution graph are correspondingly 0.75 and 1.15. Further, the spread of the distributions is relatively narrow, with d(90) values of 2.0 μm and 2.51 μm respectively, which indicates that substantially all of the powder by mass is less than 3 μm and, in the case of the heparin, less than 2 μm. Hepatin shows a smaller particle size and size distribution than clomipramine hydrochloride, probably due to lower concentration in solution.

Approximately 3 mg and 5 mg of the heparin formulation and 2 mg of the clomipramine hydrochloride formulation were then loaded and sealed into foil blisters. These were then fired from an Aspirair device into a Next Generation Impactor (NGI) with air flow set at 90 l/ml. The results for the heparin are based upon a cumulative of 5 fired blisters. Only 1 blister shot was fired for each clomipramine hydrochloride NGI.

Approximately 20 mg of the heparin or the clomipramine hydrochloride formulations were loaded and sealed into size 3 capsules. The clomipramine hydrochloride capsules were gelatine capsules and the capsules used for the heparin formulation were HPMC capsules (hydroxypropylmethyl cellulose). These capsules were then fired using the MonoHaler device into a NGI with an air flow set at 90 l/min.

The performance data are summarised as follows, the data being an average of 2 or 3 determinations:

TABLE 25
Powder performance study of drug and 5% leucine dispensed
using Aspirair (trade mark)

| | MD | DD | FPD | FPF % | FPF % | FPF % | FPF % |
|---|---|---|---|---|---|---|---|
| Aspirair | (μm) | (μm) | (μm) | (<5 μm) | (<3 μm) | (<2 μm) | (<1 μm) |
| Heparin | 1969 | 1870 | 1718 | 92 | 83 | 69 | 39 |
| 3 mg | | | | | | | |
| Heparin | 3560 | 3398 | 3032 | 89 | 78 | 60 | 31 |
| 5 mg | | | | | | | |
| Clomip- | 1739 | 1602 | 1461 | 91 | 81 | 62 | 28 |
| ramine | | | | | | | |
| 2 mg | | | | | | | |

TABLE 26
Powder performance study of drug and 5% leucine dispensed
using Aspirair (trade mark)

| | | Recovery | Throat | Blister | Device |
|---|---|---|---|---|---|
| Aspirair | MMAD | (%) | (%) | (%) | (%) |
| Heparin 3 mg | 1.30 | 65 | 5 | 2 | 2 |
| Heparin 5 mg | 1.57 | 71 | 6 | 2 | 2 |
| Clomipramine | 1.56 | 88 | 4 | 3 | 5 |
| 2 mg | | | | | |

TABLE 27
Powder performance study of drug and 5% leucine dispensed
using Monohaler (trade mark)

| | MD | DD | FPD | FPF % | FPF % | FPF % | FPF % |
|---|---|---|---|---|---|---|---|
| Monohaler | (μm) | (μm) | (μm) | (<5 μm) | (<3 μm) | (<2 μm) | (<1 μm) |
| Heparin | 14201 | 12692 | 10597 | 83 | 70 | 54 | 29 |
| 20 mg | | | | | | | |
| Clomipramine 20 mg | 18359 | 16441 | 12685 | 77 | 56 | 37 | 19 |

TABLE 28
Powder performance study of drug and 5% leucine dispensed
using Monohaler (trade mark)

| | | Recovery | Throat | Blister | Device |
|---|---|---|---|---|---|
| Monohaler | MMAD | (%) | (%) | (%) | (%) |
| Heparin | 1.72 | 70 | 6 | 5 | 6 |
| 20 mg | | | | | |
| Clomipramine | 2.38 | 86 | 10 | 1 | 9 |
| 20 mg | | | | | |

The device retention in the Aspirair device was surprisingly low (between 2-5%) for both drug formulations. This was especially low given the small particle sizes used and the relatively high dose loadings used: for example the clomipramine hydrochloride exhibited device retention in the Aspirair device of 5% and a small d(50) of 1.1 μm. In comparison, clomipramine hydrochloride co-jet milled with 5% leucine with a d(50) of 0.95 μm gave a device retention of 23% under otherwise similar circumstances. Heparin gave very low device retention in Aspirair with a d(50) of 0.8 μm and there did not appear to be a difference in device retention using the 3 mg or 5 mg filled blisters.

When using the Monohaler device to dispense the formulations, the device retention was higher than observed when the Aspirair device was used. However, device retention of respectively 6% for heparin and 9% for clomipramine hydrochloride still appears to be relative low for a formulation that comprises >90% ultrafine drug.

Throat retention was also very low for both drug formulations. When the formulations were dispensed using the Aspirair, it was as low as 4%. With Monohaler as the device, the results show slightly higher throat retention (between 6-10%).

It has previous been argued that as particle size was reduced, powder surface free energy and hence powder adhesivity and cohesivity would increase. This would be expected to result in increased device retention and poor dispersion. Such adhesivity and cohesivity and hence device retention/poor performance has been shown to be reduced by addition of force control agents, attached to the drug particle surface (or drug and excipients as appropriate). In Aspirair, it is believed that a level of adhesivity and cohesivity is desirable to prolong lifetime in the vortex, yielding a slower plume, but adhesivity and cohesivity should not be so high as to result in high device retention. Consequently a balance of particle size, adhesivity and cohesivity is believed to be requited to achieve an optimum performance in Aspirair.

The dispersion results for both powders were also excellent when using Monohaler as the device.

It is believed that the results indicate that the ultrasonic nebulising process results in a most effective relative enrichment of leucine concentration at the particle surface. The surface enrichment is dependent upon the rate of leucine transport to the surface, the size of the particle, and its precipitation rate, during the drying process. This precipitation rate is related to the slow drying of the particles in this process. The resulting effect is that the particle surface is dominated by the hydrophobic aspects of the leucine. This presents a relatively low surface energy of the powder despite its small particle size and high surface area. It therefore appears that the addition of a force control agent is having a superior influence to adhesivity and cohesivity and hence the device retention and dispersion.

The inclusion of leucine appears to provide significant improvements to the aerosolisation of heparin and clomipramine hydrochloride, and should make both drugs suitable for use in a high-dose passive or active device.

From the results presented herein, it can be seen that improvement in the FPF of spray dried active agents can be achieved by using one or more of the following:
1) tailored co-spray drying the active agent with a force control agent;
2) using a means of producing droplets for spray drying which results in slow velocity droplets, the size of which can be accurately controlled; and
3) moisture profiling of the spray dried particles.

The above discussion and experiments focussed on conventional spray drying apparatus and ultrasonic nebulizing apparatus. However, it should be noted that further changes to the apparatus may be made to ensure that the particles collected at the end of the spray drying process have the optimum properties.

For example, the nature of the drying chamber may be changed, to get better drying and/or other advantages. Thus, in one embodiment of the invention, a spray drying apparatus comprising a drying chamber with heated walls may be used. Such drying chambers are known and they have the advantage that the hot walls discourage deposition of the spray dried material on them. However, the heated walls create a temperature gradient within the drying chamber, where the air in the outer area of the chamber is hotter than that in the centre of the chamber. This uneven temperature can cause problems because particles which pass through different parts of the drying chamber will have slightly different properties as they may well dry to differing extents.

In an alternative embodiment, the spray drying apparatus comprises a radiative heat source in the drying chamber. Such heat sources are not currently used in spray drying. This type of heat source has the advantage that it does not waste energy heating the air in the drying chamber. Rather, only the droplets/particles are heated as they pass through the chamber. This type of heating is more even, avoiding the temperature gradients mentioned above in connection with drying chambers with heated walls. This also allows the particles to dry from inside the droplets thus reducing or avoiding crust forming.

In yet another embodiment, the spray dried particles are collected using a vertical drying column. These columns are already known in spray drying devices and they collect the spray dried particles by carrying the particles up a vertical column using an air flow, rather than simply relying on gravity to collect the particles in a collection chamber. The advantage of using such a vertical drying column to collect the spray dried particles is that it allows for aerodynamic classification of the particles. Fine particles tend to be carried well by the air flow, whilst larger particles are not. Therefore, the vertical drying column does not collect these larger particles.

In view of the increased FPF and FPD obtained, especially when co-spray drying an active agent with an FCA, it may be possible to do away with the large carrier particles in a dry powder comprising an active agent which has been co-spray dried with a force control agent. However, it may still be desirable to include carrier particles, especially where the active agent is to be administered in small amounts, as the bulk of the larger carrier particles will help to ensure that an accurate dose is dispensed.

Whilst any of the abovementioned active agents can be spray dried as discussed above, preferably, the active agent is a small molecule, as opposed to a macromolecule. Preferably, the active agent is not a protein, and more preferably, the active agent is not insulin. In the case of proteins and in particular insulin, there is little or no benefit to be derived from the use of a force control agent in a dry powder formulation for administration by inhalation. The reason for this is that in the case of these active agents, the active agent itself acts as a force control agent and the cohesive forces of particles of these active agents are already only weak.

As discussed above, where the active agent being spray dried includes hydrophobic moieties itself, it is possible to spray dry the active agent without an FCA.

The active agent, preferably, exhibits-greater than 20, 25, 30, and, more preferably, 40% bio-availability when administered via the lung in the absence of a penetration enhancer. Tests suitable for determining bio-availability are well known to those skilled in the art and an example is described in WO 95/00127. Agents that exhibit bio-availability of less than 20%, such as a majority of macromolecules, are insufficiently rapidly cleared from the deep lung and, as a result, accumulate to an unacceptable extent if administered to this location on a long term basis.

It is thought that the bio-availability of the active agent may be improved by delivering the active agent to the lung in particles with a size of less than 2 μm, less than 1.5 μm or less than 1 μm. Thus, the spray dried particles of the present invention, which tend to have a particle size of between 0.5 and 5 μm will exhibit excellent bio-availability compared to that of the particles produced by conventional spray drying processes.

It is important to note that the particles produced by co-spray drying an active agent and an FCA will comprise both the active agent and the FCA and so the FCA will actually be administered to the lower respiratory tract or deep lung upon inhalation of the dry powder composition. This is in contrast to the additive material used in the prior art, which often was not administered to the deep lung, for example because it remains attached to the large carrier particles.

Thus, it is important that the selected FCA does not have a detrimental effect when administered to the lower respiratory tract or deep lung. Amino acids such as leucine, lysine and cysteine are all harmless in this regard, as are other FCAs such as phospholipids, when present in small quantities.

Micronised Dry Powder Particles

In another aspect of the present invention, a method of producing powders is provided wherein the method achieves a further reduction in the size of the active particles, preferably so that the particles are of an appropriate size for administration to the deep lung by inhalation. Preferably, this is possible using both active dry powder inhaler devices and passive dry powder inhaler devices.

In particular, the present invention seeks to optimise the preparation of particles of active agent used in the dry powder composition by engineering the particles making up the dry powder composition and, in particular, by engineering the particles of active agent. It is proposed to do this by adjusting and adapting the milling process used to form the particles of active agent.

According to an aspect of the present invention, a method is provided for making composite active particles for use in a pharmaceutical composition for pulmonary inhalation, the method comprising jet milling active particles in the presence of additive material, preferably wherein the jet milling is conducted using air or a compressible gas or fluid.

In the conventional use of the word, “milling” means the use of any mechanical process which applies sufficient force to the particles of active material that it is capable of breaking coarse particles (for example, particles with a MMAD greater than 100 μm) down to fine particles (for example, having a MMAD not more than 50 μm). In the present invention, the term “milling” also refers to deagglomeration of particles in a formulation, with or without particle size reduction. The particles being milled may be large or fine prior to the milling step.

In the prior art, co-milling or co-micronising active agents and additive materials have been suggested. It is stated that milling can be used to substantially decrease the size of particles of active agent. However, if the particles of active agent are already fine, for example have a MMAD of less than 20 μm prior to the milling step, the size of those particles may not be significantly reduced where the milling of these active particles takes place in the presence of an additive material. Rather, milling of fine active particles with additive particles using the methods described in the prior art (for example, in WO 02/43701) will result in the additive material becoming deformed and being smeared over or fused to the surfaces of the active particles. The resultant composite active particles have been found to be less cohesive after the milling treatment. However, there is still the disadvantage that this is not combined with a significant reduction in the size of the particles.

The prior art mentions two types of processes in the context of co-milling or co-micronising active and additive particles.

First, there is the compressive type process, such as Mechano-Fusion and Cyclomix methods. As the name suggests, Mechano-Fusion is a dry coating process designed to mechanically fuse a first material onto a second material. The first material is generally smaller and/or softer than the second. The Mechano-Fusion and Cyclomix working principles are distinct from alternative milling techniques in having a particular interaction between an inner element and a vessel wall, and are based on providing energy by a controlled and substantial compressive force.

The fine active particles and the additive particles are fed into the Mechano-Fusion driven vessel (such as a Mechano-Fusion system (Hosokawa Micron Ltd)), where they are subject to a centrifugal force and are pressed against the vessel inner wall. The powder is compressed between the fixed clearance of the drum wall and a curved inner element with high relative speed between drum and element. The inner wall and the curved element together form a gap or nip in which the particles are pressed together. As a result, the particles experience very high shear forces and very strong compressive stresses as they are trapped between the inner drum wall and the inner element (which has a greater curvature than the inner drum wall). The particles are pressed against each other with enough energy to locally heat and soften, break, distort, flatten and wrap the additive particles around the core particle to form a coating. The energy is generally sufficient to break up agglomerates and some degree of size reduction of both components may occur.

These Mechano-Fusion and Cyclomix processes apply a high enough degree of force to separate the individual particles of active material and to break up tightly bound agglomerates of the active particles such that effective mixing and effective application of the additive material to the surfaces of those particles is achieved. An especially desirable aspect of the described co-milling processes is that the additive material becomes deformed in the milling and may be smeared over or fused to the surfaces of the active particles.

However, in practice, this compression process produces little or no milling (i.e. size reduction) of the drug particles, especially where they are already in a micronised form (i.e. <10 μm), the only physical change which may be observed is a plastic deformation of the particles to a rounder shape.

Secondly, there are the impact milling processes involved in ball milling and the use of a homogenizer.

Ball milling is a suitable milling method for use in the prior art co-milling processes. Centrifugal and planetary ball milling are especially preferred methods. Alternatively, a high pressure homogeniser may be used in which a fluid containing the particles is forced through a valve at high pressure producing conditions of high shear and turbulence. Such homogenisers may be more suitable than ball mills for use in large scale preparations of the composite active particles.

Suitable homogenisers include EmulsiFlex high pressure homogenisers which are capable of pressures up to 4000 bar, Niro Soavi high pressure homogenisers (capable of pressures up to 2000 bar), and Microfluidics Microfluidisers (maximum pressure 2750 bar). The milling step may, alternatively, involve a high energy media mill or an agitator bead mill, for example, the Netzsch high energy media mill, or the DYNO-mill (Willy A. Bachofen AG, Switzerland).

These processes create high-energy impacts between media and particles or between particles. In practice, while these processes are good at making very small particles, it has been found that neither the ball mill not the homogenizer was effective in producing dispersion improvements in resultant drug powders in the way observed for the compressive process. It is believed that the second impact processes are not as effective in producing a coating of additive material on each particle.

Conventional methods comprising co-milling active material with additive materials (as described in WO 02/43701) result in composite active particles which are fine particles of active material with an amount of the additive material on their surfaces. The additive material is preferably in the form of a coating on the surfaces of the particles of active material. The coating may be a discontinuous coating. The additive material may be in the form of particles adhering to the surfaces of the particles of active material.

At least some of the composite active particles may be in the form of agglomerates. However, when the composite active particles are included in a pharmaceutical composition, the additive material promotes the dispersal of the composite active particles on administration of that composition to a patient, via actuation of an inhaler.

Jet mills are capable of reducing solids to particle sizes in the low-micron to submicron range. The grinding energy is created by gas streams from horizontal grinding air nozzles. Particles in the fluidized bed created by the gas streams are accelerated towards the centre of the mill, colliding with slower moving particles. The gas streams and the particles carried in them create a violent turbulence and as the particles collide with one another they are pulverized.

In the past, jet-milling has not been considered attractive for co-milling active and additive particles, processes like Mechano-Fusion and Cyclomixing being clearly preferred. The collisions between the particles in a jet mill are somewhat uncontrolled and those skilled in the art, therefore, considered it unlikely for this technique to be able to provide the desired deposition of a coating of additive material on the surface of the active particles. Moreover, it was believed that, unlike the situation with Mechano-Fusion and Cyclomixing, segregation of the powder constituents occurred in jet mills, such that the finer particles, that were believed to be the most effective, could escape from the process. In contrast, it could be clearly envisaged how techniques such as Mechano-Fusion would result in the desired coating.

It should also be noted that it was also previously believed that the compressive or impact milling processes must be carried out in a closed system, in order to prevent segregation of the different particles. This has also been found to be untrue and the co-jet milling processes according to the present invention do not need to be carried out in a closed system. Even in an open system, the co-jet milling has surprisingly been found not to result in the loss of the small particles, even when using leucine as the additive material.

It has now unexpectedly been discovered that composite particles of active and additive material can be produced by co-jet milling these materials. The resultant particles have excellent characteristics which lead to greatly improved performance when the particles are dispensed from a DPI for administration by inhalation. In particular, co-jet milling active and additive particles can lead to further significant particle size reduction. What is more, the composite active particles exhibit an enhanced FPD and FPF, compared to those disclosed in the prior art.

The effectiveness of the promotion of dispersal of active particles has been found to be enhanced by using the co-jet milling methods according to the present invention in comparison to compositions which are made by simple blending of similarly sized particles of active material with additive material. The phrase “simple blending” means blending or mixing using conventional tumble blenders or high shear mixing and basically the use of traditional mixing apparatus which would be available to the skilled person in a standard laboratory.

It has been found that, contrary to previous belief, co-jet milling can be used to produce sufficiently complete coatings of additive material, which have now been observed to substantially improve the dispersion of the powders from an inhaler. The jet milling process can also be adjusted to tailor the composite particles to the type of inhaler device to be used to dispense the particles. The inhaler device may be an active inhaler device, such as Aspirair™ or it may be a passive device.

Further, the co-jet milling process may optionally also be arranged so as to significantly mill the active particles, that is, to significantly reduce the size of the active particles. The co-jet milling of the present invention may even, in certain circumstances, be more efficient in the presence of the additive material than it is in the absence of the additive material. The benefits are that it is therefore possible to produce smaller particles for the same mill, and it is possible to produce milled particles with less energy. Co-jet milling should also reduce the problem of amorphous content by both creating less amorphous material, as well as hiding it below a layer of additive material.

The impact forces of the co-jet milling are sufficient to break up agglomerates of drug, even micronised drug, and are effective at distributing the additive material to the consequently exposed faces of the particles. This is an important aspect of the present invention. It has been shown that if the energy of the process is not sufficient to break up the agglomerates of drug (for example, as will be the case when one uses a conventional blender), the additive material merely coats the agglomerates and these agglomerates can even be compressed, making them even more difficult to disperse. This is clearly undesirable when one is seeking to prepare a dry powder for administration by inhalation.

Fine particles of active material suitable for pulmonary administration have often been prepared by milling in the past. However, when using many of the known milling techniques, once the particles reach a minimum size, referred to as the “critical size”, they tend to re-combine at the same rate as being fractured, or do not fracture effectively and therefore no further reduction in the particle size is achieved. Critical sizes are specific to particular mills and sets of milling conditions.

Thus, manufacture of fine particles by milling can require much effort and there are factors which consequently place limits on the minimum size of particles of active material which can be achieved, in practice, by such milling processes.

The present invention consequently relates to the provision of a high-energy impact process that is effective in producing improvements in the resultant drug powders.

Furthermore, contrary to conventional thinking, the processes of the present invention do not need to be carried out in a closed system. Even where the additive material being co-jet milled is leucine, there is no observed loss of additive material or reduction in coating where the jet-milling is not carried out in a closed system. Rather, in one embodiment of the invention, the method of the present invention is carried out in a flow-through system, without any loss in performance of the resultant composite particles. This is an economically important feature, as it can significantly increase the rate of production of the powders of the invention.

In one embodiment of the present invention, 90% by mass of the active particles jet-milled are initially less than 20 μm in diameter. More preferably, 90% by mass of the active particles jet-milled are initially less than 10 μm in diameter, and most preferably less than 5 μm in diameter.

In another embodiment, 90% by mass of the additive particles jet-milled are initially less than 20 μm in diameter. More preferably, 90% by mass of the additive particles jet-milled are initially less than 10 μm in diameter, and most preferably less than 5 μm in diameter or less than 3 μm in diameter.

The terms “active particles” and “particles of active material” and the like are used interchangeably herein. The active particles comprise one or more pharmaceutically active agents.

Preferably, the active agent is a small molecule, as opposed to a macromolecule. Preferably, the active agent is not a protein, and more preferably, the active agent is not insulin. In the case of proteins and in particular insulin, there is little or no benefit to be derived from the use of a force control agent in a dry powder formulation for administration by inhalation. The reason for this is that in the case of these active agents, the active agent itself acts as a force control agent and the cohesive forces of particles of these active agents are already only weak.

In preferred embodiments of the present invention, the active agent is heparin, apomorphine, clobozam, clomipramine or glycopyrrolate; The terms “additive particles” and “particles of additive material” are used interchangeably herein. The additive particles comprise one or more additive materials (or FCAs). Preferably, the additive particles consist essentially of the additive material.

Suitable additive materials for use in the milling methods disclosed herein are listed above (as FCAs).

In general, the optimum amount of additive material to be included in a dry powder formulation will depend on the chemical composition and other properties of the additive material and of the active material, as well as upon the nature of other particles, such as carrier particles, if present. In general, the efficacy of the additive material is measured in terms of the FPF of the composition.

In one embodiment of the present invention, composite active particles produced by co-jet milling according to the present invention are mixed with carrier particles made of an inert excipient material.

Where the powder composition comprises an active material, additive material and excipient material, this is referred to as a 3-component system. In contrast, a 2-component system comprises just active and additive materials.

Excipient materials may be included in powders for administration by pulmonary inhalation for a number of reasons. On the one hand, the inclusion of particles of excipient material of an appropriate size can enhance the flow properties of the powder and can enhance the powder's handleability. Excipient material is also added to powder formulations as a diluent. It can be very difficult to accurately and reproducibly administer a very small amount of powder. Where low doses of drug are required, this can pose a problem and so it can be desirable to add a diluent to the powder, to increase the amount of powder to be dispensed.

In one embodiment of the present invention, the excipient material is in the form of relatively large or coarse carrier particles. Advantageously, substantially all (by weight) of the carrier particles have a diameter which lies between about 20 μm and about 1000 μm, more preferably about 50 μm and about 1000 μm. Preferably, the diameter of substantially all (by weight) of the carrier particles is less than about 355 μm and lies between about 20 μm and about 250 μm.

Preferably at least about 90% by weight of the carrier particles have a diameter between from about 60 μm to about 180 μm. The relatively large diameter of the carrier particles improves the opportunity for other, smaller particles to become attached to the surfaces of the carrier particles and provides good flow and entrainment characteristics and improved release of the active particles in the airways to increase deposition of the active particles in the lung.

Conventional thinking regarding carrier particles is that they improve the poor flowability of formulations comprising fine particles of less than 10 μm. The poor flowability is due to the agglomeration of the fine particles which occurs due to the strong attractive forces between the small particles. In the presence of large carrier particles, these attractive forces cause the fine particles to become attached to the surface of the large carrier particles, forming (usually discontinuous) coatings. This arrangement of the large and fine particles leads to better flow characteristics than is observed with a formulation made up solely of fine active particles.

The carrier particles to be added to the composite active particles of the present invention are relatively large particles of an excipient material, such as lactose.

The ratios in which the carrier particles and composite active particles are mixed will, of course, depend on the type of inhaler device used, the type of active particles used and the required dose. The carrier particles may be present in an amount of at least about 50%, more preferably at least about 70%, more preferably at least about 80%, advantageously at least about 90% and most preferably at least about 95%, based on the combined weight of the composite active particles and the carrier particles.

A 3-component system including carrier particles, such as the one described above, would be expected to work well in a passive device. The presence of the carrier particles makes the powder easier to extract from the blister, capsule or other storage means. The powder extraction tends to pose more of a problem in passive devices, as they do not create as turbulent an air flow through the blister upon actuation as active devices. This means that it can be difficult to entrain all of the powder in the air flow. The powder entrainment in a passive device is made easier where the powder includes carrier particles as this will mean that the powder is less cohesive and exhibits better flowability, compared with a powder consisting entirely of smaller particles, for example all having a diameter of less than 10 μm.

Where carrier particles and the composite active particles made according to the present invention are mixed, the active particles should readily release from the surface of the carrier particles upon actuation of the dispensing device by virtue of the additive material on the surface of the active particles. This release may be further improved where the carrier particles also have additive material applied to their surfaces. This application can be achieved by simple gentle blending or co-milling, for example as described in WO 97/03649.

However, the combination of large carrier particles and fine active particles has its disadvantages. It can only be effectively used with a relatively low (usually only up to 5%) drug content. As greater proportions of fine particles are used, more and more of the fine particles fail to become attached to the large carrier particles and segregation of the powder formulation becomes a problem. This, in turn, can lead to unpredictable and inconsistent dosing. The powder also becomes more cohesive and difficult to handle.

Furthermore, the size of the carrier particles used in a dry powder formulation can be influential on segregation.

Segregation can be a catastrophic problem in powder handling during manufacture and the filling of devices or device components (such as capsules or blisters) from which the powder is to be dispensed. Segregation tends to occur where ordered mixes cannot be made sufficiently stable. Ordered mixes occur where there is a significant disparity in powder particle size. Ordered mixes become unstable and prone to segregation when the relative level of the fine component increases beyond the quantity which can adhere to the larger component surface, and so becomes loose and tends to separate from the main blend. When this happens, the instability is actually exacerbated by the addition of anti-adherents/glidants such as FCAs.

In the case of dry powder formulations of micron-sized drug, and typical 60 to 150 μm sized carrier, this instability tends to occur once drug content exceeds a few percent, the exact amount is dependant on the drug. However, it has been found that a carrier with a particle size of <30 μm tends not to exhibit this instability. This is thought to be due to the fine carrier particles having relatively higher surface area compared to the coarse carrier particles, and the similarity between the size of the active particles and the carrier particles. Such fine carrier particles are not often used, mainly because of their poor flow characteristics, as discussed above.

According to another embodiment of the present invention, the 3-component system comprises the composite active particles made according to the present invention, together with fine excipient particles. Such excipient particles have a particle size of 30 μm or less, preferably 20 μm or less and more preferably 10 μm or less. The excipient particles advantageously have a particle size of 30 to 5 μm.

One would expect such a powder formulation, made up of only fine particles with a particle size of less than 10, to suffer from the cohesion and flowability problems observed with formulations comprising just fine active particles. The active particles do not coat the fine excipient particles, as they do the large carrier particles, because of the different forces existing between fine particles and fine and large particles.

However, where the powder formulation comprises composite active particles according to the present invention and fine excipient particles, it has been surprisingly found that such formulations are efficiently dispensed by an active device. It has been found that the potentially poor flow characteristics or handleability of powders comprising only particles with a size of less than 10 μm are not significant when the powder is dispensed using an active inhaler device.

As mentioned above, the active device causes turbulence within the blister, capsule or other powder storage means. This means that even powders with fine excipient particles can be extracted. Furthermore, the presence of the composite active particles means that the agglomerates formed from the fine particles are not so stable that they are not broken up upon actuation of the inhaler device. Thus, it has been surprisingly found that compositions comprising the composite active particles of the present invention and fine particles of an inert excipient material, such as lactose, can be efficiently dispensed using an active inhaler device.

In another embodiment of the present invention, the fine excipient particles added to the composite active particles are themselves co-jet milled with additive material. The co-jet milling of the active particles with additive material and of the excipient particles with additive material can occur separately or together.

Co-jet milling the fine excipient particles with the additive material results in coating of the additive material on the surfaces of the excipient particles. This coating can further reduce the cohesiveness of the 3-component system and can further enhance deagglomeration upon actuation of the inhaler device.

Generally, flow of compositions comprising fine carrier particles is poor unless they are pelletised (e.g. as is done in the AstraZeneca product OXIS (registered trade mark). However, using the processes of the present invention, fine lactoses (e.g. Sorbolac 400 with a particle size of 1 to 15 μm) have been produced which flow sufficiently well for use in DPIs with >5% drug, and up to approximately 30% and possibly 50% cohesive micronised drug. It should be noted that these beneficial properties are achieved without the need to resort to pelletisation, which has its own disadvantages of being difficult to do and generally decreasing FPFs.

Thus, the co-milling of the fine excipient particles and additive material in accordance with the present invention allows one to produce blends of active and excipient materials with a much greater range of active agent content than is possible using conventional carrier particles (i.e. >5%). The resultant dry powder formulations also benefit from improved aerosolisation.

In the present invention, different grinding and injection pressures may be used in order to produce particles with different coating characteristics. The invention also includes embodiments where different grinding and injection pressures are combined, to produce composite particles with desired properties, that is, to engineer the particles.

Co-jet milling may be carried out at grinding pressures between 0.1 and 12 bar. Varying the pressure allows one to control the degree of particle size reduction. At pressures in the region of 0.1-3 bar, and preferably 1-2 bar, the co-jet milling will primarily result in blending of the active and additive particles, so that the additive material coats the active particles. On the other hand, at 3-12 bar, and preferably 5-12 bar, the co-jet milling will additionally lead to particle size reduction.

In one embodiment, the jet milling is carried out at a grinding pressure of between 0.1 and 3 bar, to achieve blending of the active and additive particles. As discussed below in greater detail, when the co-jet milling of the present invention is carried out at such relatively low pressures, the resultant particles have been shown to perform well when dispensed using passive devices. It is speculated that this is because the particles are larger than those produced by co-jet milling at higher pressures and these relatively larger particles are more easily extracted from the blister, capsule or other storage means in the passive device, due to less cohesion and better flowability. Whilst such relatively large particles are easily extracted from the blister or capsule in an active device, they may result in throat deposition.

In another embodiment, the jet milling is carried out at a grinding pressure of between 3 and 12 bar, to achieve reduction of the sizes of the active and additive particles. The co-jet milling at these relatively high pressures can produce extremely small composite active particles having a MMAD of between 3 and 0.5 μm. These fine particle sizes are excellent for deep lung deposition, but they really need to be dispensed using an active inhaler device, as the powder formulations comprising such fine particles are actually rather “sticky”. As discussed below, this stickiness does not pose a problem for active devices and is actually thought to be advantageous as it can slow the extraction of the powder so that the composite active particles travel more slowly in the powder plume generated by the device, thereby reducing throat deposition.

Tests were carried out whereby pre-micronised lactose (as a drug model) was co-jet milled in an MC50 Hosakawa Micron with 5% magnesium stearate. At 2 bar milling pressure, the resultant material had a d50 of approximately 3 μm, whilst milling the same mixture at around 7 bar resulted in material with a d50 of about 1 μm. Thus, when operating with a jet milling pressure of 0.1-3 bar little milling, that it is particle size reduction, is seen. From 3-12 bar milling pressure, increasing milling is seen, with the particle size reduction increasing with the increasing pressure. This means that the milling pressure may be selected according to the desired particle size in the resultant mixture.

As indicated above, co-jet milling at lower pressures produces powders which perform well in passive devices whilst powders milled at higher pressures perform better in active devices, such as Aspirair™.

The co-jet milling processes according to the present invention can also be carried out in two or more stages, to combine the beneficial effects of the milling at different pressures and/or different types of milling or blending processes. The use of multiple steps allows one to tailor the properties of the co-jet milled particles to suit a particular inhaler device, a particular drug and/or to target particular parts of the lung.

In one embodiment, the milling process is a two-step process comprising first jet-milling the drug on its own at high grinding pressure to obtain the very small particle sizes possible using this type of milling. Next, the milled drug is co-jet milled with an additive material. Preferably, this second step is carried out at a lower grinding pressure, so that the effect is the coating of the small active particles with the additive material.

This two-step process produces better results than simply co-jet milling the active material and additive material at a high grinding pressure. Experimental results discussed below show that the two-step process results in smaller particles and less throat deposition than simple co-jet milling of the materials at a high grinding pressure.

In another embodiment of the present invention, the particles produced using the two-step process discussed above subsequently undergo Mechano-Fusion. This final Mechano-Fusion step is thought to “polish” the composite active particles, further rubbing the additive material into the particles. This allows one to enjoy the beneficial properties afforded to particles by Mechano-Fusion, in combination with the very small particles sizes made possible by the co-jet milling.

The reduction in particle size may be increased by carrying out the co-jet milling at lower temperatures. Whilst the co-jet milling process may be carried out at temperatures between −20° C. and 40° C., the particles will tend to be more brittle at lower temperatures, and they therefore fracture more readily so that the milled particles tend to be even smaller. Therefore, in another embodiment, the jet milling is carried out at temperatures below room temperature, preferably at a temperature below 10° C., more preferably at a temperature below 0° C.

Preferably, all of the particles are of a similar size distribution. That is, substantially all of the particles are within the size range of about 0 to about 50 μm, of about 0 to about 20 μm, of about 0 to 10 μm, of about 0 to 5 μm or of about 0 to 2 μm.

In accordance with a second-aspect of the present invention, a pharmaceutical dry powder composition for pulmonary inhalation is provided, comprising composite active particles made by a method according to the first aspect of the invention.

The MMAD of the composite active particles is preferably not more than 10 μm, and advantageously it is not more than 5 μm, more preferably not more than 3 μm, even more preferably not more than 2 μm, more preferably not more than 1.5 μm, even more preferably not more than 1.2 μm and most preferably not more than 1 μm.

Accordingly, advantageously at least 90% by weight of the composite active particles have a diameter of not more than 10 μm, advantageously not more than 5 μm, preferably not more than 3 μm, even more preferably not more than 2.5 μm, even more preferably not more than 2 μm and more preferably not more than 1 μm.

In a preferred embodiment of the present invention the resultant dry powder formulation has a reproducible FPF(ED) of at least 70%. Preferably, the FPF(ED) will be at least 80%, more preferably the FPF(ED) will be at least 85%, and most preferably the FPF(ED) will be at least 90%.

In a further preferred embodiment, the dry powder formulation has a reproducible FPF(MD) of at least 60%. Preferably, the FPF(MD) will be at least 70%, more preferably the FPF(MD) will be at least 80%, and most preferably the FPF(MD) will be at least 85%.

As illustrated by the experimental results set out below, it has been surprisingly found that co-milling active particles with additive particles using jet milling results in composite active particles having significantly better FPF and FPD than those produced by co-milling using Mechano-Fusion, when the powders are dispensed using the active inhaler device Aspirair™.

This unexpected improvement in the FPF and FPD of the powder formulations prepared is thought to be due to the following factors. Firstly, the milling process results in very small particles. Secondly, there appears to be only partial coverage of the particles with the force control agent and this means that some of the particle cohesion is retained, affording better powder handleability despite the very small particle sizes.

Co-jet milling has surprisingly been found to be capable of significantly reducing the median primary particle size of active particles (for example, from 3 or 2 μm to 1 m), while also allowing good aerosolisation from a delivery device. This further reduction in primary particle size is considered to be advantageous for delivery of systemically targeted molecules to the deep lung. The benefit here is to co-jet mill active particles with additive particles in order to reduce primary particle size while still achieving a reduction in the level of powder cohesion and adhesion by coating the particles for additive material.

Test Methods

All materials were evaluated in the Next Generation Impactor (NGI). Details of the test are provided in each case.

Formulations were processed using:

1) The Hosokawa Micron Mechano-Fusion AMS Mini system. This system was operated with a novel rotor, providing a 1 mm compression gap; and

2) The Hosokawa Micron AS50 spiral jet mill.

The in-vitro testing was performed using an Aspirair™ device, which is an active inhaler device.

The formulations were composed of one or more of the following constituents:

Magnesium stearate (standard grade)

L-Leucine (Ajinomoto) and jet milled by Micron Technologies

Sorbolac 400 lactose

Micronised clobozam

Micronised apomorphine hydrochloride

Micronised lactose

Re-condensed Leucine (Aerocine)

Comparison of Co-Jet Milled and Mechano-Fused Formulations (Clobozam)

The following is a comparison of 2-component systems comprising co-jet milled or Mechano-Fused active particles and additive material.

1.01 g of micronised clobozam was weighed out, and then gently pressed through a 300 μm metal sieve, using the rounded face of a metal spatula. This formulation was recorded as “3A”.

9.37 g of micronised clobozam was then combined with 0.50 g of micronised L-leucine in the Mechano-Fusion system. The material was processed at a setting of 20% power for 5 minutes, followed by a setting of 80% power for 10 minutes. This material was recorded as “4A”. After blending, this powder was then gently pushed through a 300 μm metal sieve with a spatula. This material was recorded as “4B”. 9.57 g of micronised clobozam was then combined with 0.50 g of magnesium stearate in the Mechano-Fusion system. The material was processed at a setting of 20% power for 5 minutes, followed by a setting of 80% power for 10 minutes. This material was recorded as “5A”. After blending, this powder was rested overnight, and then was gently pushed through a 300 μm metal sieve with a spatula. This material was recorded as “5B”.

9.5 g of micronised clobozam was then combined with 0.50 g of micronised L-leucine in the Mechano-Fusion system. The material was processed at a relatively low speed setting of 20% power for 5 minutes. This process was intended only to produce a good mix of the components. This material was recorded as “6A”.

6.09 g of “6A” fed at approximately 1 g per minute into an AS50 spiral jet mill, set with an injector pressure of about 7 bar and a grinding pressure of about 5 bar. The resulting material was recovered and recorded as “6B”.

After milling, this powder was rested overnight, and then was gently pushed through a 300 μm metal sieve with a spatula. This material was recorded as “6C”.

9.5 g of micronised clobozam was then combined with 0.50 g of magnesium stearate in the Mechano-Fusion system. The material was processed at a setting of 20% power for 5 minutes. This material was recorded as “7A”.

6.00 g of “7A” was fed at approximately 1 g per minute into the AS50 spiral jet mill, set with an injector pressure of about 7 bar and a grinding pressure of about 5 bar. The resulting material was recovered and recorded as “7B”.

After milling, this powder was gently pushed through a 300 μm metal sieve with a spatula. This material was recorded as “7C”.

A batch of re-condensed leucine (also referred to as “Aerocine”) was produced by subliming to vapour a sample of leucine in a tube furnace, and re-condensing as a very finely dispersed powder as the vapour cooled. This batch was identified as “8A”.

9.5 g of micronised clobozam was then combined with 0.50 g of Aerocine, in the Mechano-Fusion system. The material was processed at a setting of 20% power for 5 minutes, followed by a setting of 80% power for 10 minutes. This material was recorded as “8B”. After blending, this powder was rested overnight, and then was gently pushed through a 300 μm metal sieve with a spatula. This material was recorded as “8C”.

9.5 g of micronised clobozam was combined with 0.50 g of Aerocine in the Mechano-Fusion system. The material was processed at a setting of 20% power for 5 minutes. 7.00 g of this powder was then fed into the AS50 spiral jet mill, set with an injector pressure of about 7 bar and a grinding pressure of about 5 bar. The resulting material was recovered and recorded as “9A”.

After milling, this powder was gently pushed through a 300 μm metal sieve with a spatula. This material was recorded as “9B”.

A number of foil blisters were filled with approximately 2 mg of the following clobozam formulations:

3A—no milling & no additive material

4B—leucine & Mechano-Fused

5B—magnesium stearate & Mechano-Fused

6C—leucine & co-jet milled

7C—magnesium stearate & co-jet milled

8C—Aerocine & co-jet milled

9B—Aerocine & Mechano-Fused.

These formulations were then fired from an Aspirair device into an NGI at a flow rate of 60 l/m. The Aspirair was operated under 2 conditions for each formulation: with a reservoir of 15 ml of air at 1.5 bar or with a reservoir of 30 ml of air at 0.5 bar.

Full details of the results are attached. The impactor test results are summarised in Tables 29, 30 and 31 below.

TABLE 29

| | | | FPD(mg) | |
|---|---|---|---|---|
| Formulation | MD (mg) | DD (mg) | (<5 μm) | MMAD |
| 3A | 2.04 | 1.12 | 0.88 | 2.91 |
| 0.5 bar 30 ml | | | | |
| 3A | 1.92 | 1.74 | 1.23 | 2.86 |
| 1.5 bar 15 ml | | | | |
| 4B | 1.84 | 1.48 | 0.82 | 3.84 |
| 0.5 bar 30 ml | | | | |
| 4B | 1.80 | 1.56 | 0.81 | 3.32 |
| 1.5 bar 15 ml | | | | |
| 5B | 1.84 | 1.53 | 1.17 | 2.34 |
| 0.5 bar 30 ml | | | | |
| 5B | 1.85 | 1.55 | 1.12 | 2.22 |
| 1.5 bar 15 ml | | | | |
| 6C | 1.93 | 1.80 | 1.67 | 2.11 |
| 0.5 bar 30 ml | 1.86 | 1.73 | 1.62 | 2.11 |
| 6C | 1.97 | 1.86 | 1.67 | 2.07 |
| 1.5 bar 15 ml | | | | |
| 6C | 1.74 | 1.65 | 1.46 | 2.03 |
| 1.5 bar 15 ml | | | | |
| (silicon coated plates) | | | | |
| 7C | 2.06 | 1.99 | 1.87 | 1.97 |
| 0.5 bar 30 ml | | | | |
| 7C | 1.89 | 1.78 | 1.63 | 1.79 |
| 1.5 bar 15 ml | | | | |
| 8C | 1.82 | 1.73 | 1.62 | 2.02 |
| 0.5 bar 30 ml | | | | |
| 8C | 1.81 | 1.74 | 1.57 | 2.01 |
| 1.5 bar 15 ml | | | | |
| 9B | 1.88 | 1.73 | 1.04 | 3.48 |
| 0.5 bar 30 ml | | | | |
| 9B | 1.80 | 1.64 | 0.94 | 3.12 |
| 1.5 bar 15 ml | | | | |

TABLE 30

| | FPF(MD) % | FPF(ED) % | FPF(ED) % | FPF(ED) % | FPF(ED) % |
|---|---|---|---|---|---|
| Formulation | (<5 μm) | (<5 μm) | (<3 μm) | (<2 μm) | (<1 μm) |
| 3A | 43 | 78 | 49 | 32 | 17 |
| 0.5 bar 30 ml | | | | | |
| 3A | 64 | 71 | 45 | 24 | 6 |
| 1.5 bar 15 ml | | | | | |
| 4B | 45 | 55 | 28 | 15 | 7 |
| 0.5 bar 30 ml | | | | | |
| 4B | 45 | 52 | 30 | 18 | 9 |
| 1.5 bar 15 ml | | | | | |
| 5B | 64 | 77 | 54 | 42 | 30 |
| 0.5 bar 30 ml | | | | | |
| 5B | 61 | 72 | 52 | 38 | 25 |
| 1.5 bar 15 ml | | | | | |
| 6C | 87 | 93 | 77 | 44 | 8 |
| 0.5 bar 30 ml | 87 | 94 | 76 | 44 | 9 |
| 6C | 85 | 90 | 73 | 44 | 10 |
| 1.5 bar 15 ml | | | | | |
| 6C | 84 | 89 | 74 | 45 | 8 |
| 1.5 bar 15 ml | | | | | |
| (silicon coated plates) | | | | | |
| 7C | 91 | 94 | 79 | 50 | 14 |
| 0.5 bar 30 ml | | | | | |
| 7C | 86 | 92 | 82 | 56 | 16 |
| 1.5 bar 15 ml | | | | | |
| 8C | 89 | 93 | 79 | 48 | 12 |
| 0.5 bar 30 ml | | | | | |
| 8C | 87 | 90 | 76 | 46 | 9 |
| 1.5 bar 15 ml | | | | | |
| 9B | 55 | 60 | 34 | 24 | 15 |
| 0.5 bar 30 ml | | | | | |
| 9B | 52 | 57 | 34 | 24 | 15 |
| 1.5 bar 15 ml | | | | | |

TABLE 31

| Formulation | *recovery | *throat | *blister | *device |
|---|---|---|---|---|
| 3A | 102% | 3% | 1% | 43% |
| 0.5 bar 30 ml | | | | |
| 3A | 96% | 15% | 1% | 8% |
| 1.5 bar 15 ml | | | | |
| 4B | 97% | 15% | 7% | 12% |
| 0.5 bar 30 ml | | | | |
| 4B | 95% | 27% | 6% | 8% |
| 1.5 bar 15 ml | | | | |
| 5B | 97% | 7% | 13% | 4% |
| 0.5 bar 30 ml | | | | |
| 5B | 98% | 14% | 12% | 4% |
| 1.5 bar 15 ml | | | | |
| 6C | 97% | 2% | 1% | 6% |
| 0.5 bar 30 ml | 101% | 3% | 1% | 5% |
| 6C | 104% | 6% | 3% | 3% |
| 1.5 bar 15 ml | | | | |
| 6C | 91% | 8% | 1% | 4% |
| 1.5 bar 15 ml | | | | |
| (silicon coated plates) | | | | |
| 7C | 110% | 2% | 1% | 3% |
| 0.5 bar 30 ml | | | | |
| 7C | 99% | 6% | 2% | 3% |
| 1.5 bar 15 ml | | | | |
| 8C | 99% | 3% | 1% | 4% |
| 0.5 bar 30 ml | | | | |
| 8C | 95% | 6% | 1% | 3% |
| 1.5 bar 15 ml | | | | |
| 9B | 96% | 16% | 2% | 7% |
| 0.5 bar 30 ml | | | | |
| 9B | 95% | 26% | 4% | 5% |
| 1.5 bar 15 ml | | | | |

From these results it can be seen that the co-jet milled formulations exhibited exceptional FPFs when dispensed from an active dry powder inhaler device. The FPFs observed were significantly better that those of the Mechano-Fused formulations and those formulations which did not include an additive material. This improvement would appear to be largely due to reduced throat deposition, which was less than 8% for the co-jet milled formulations, compared to 15% for the pure drug and up to 27% for the Mechano-Fused formulations.

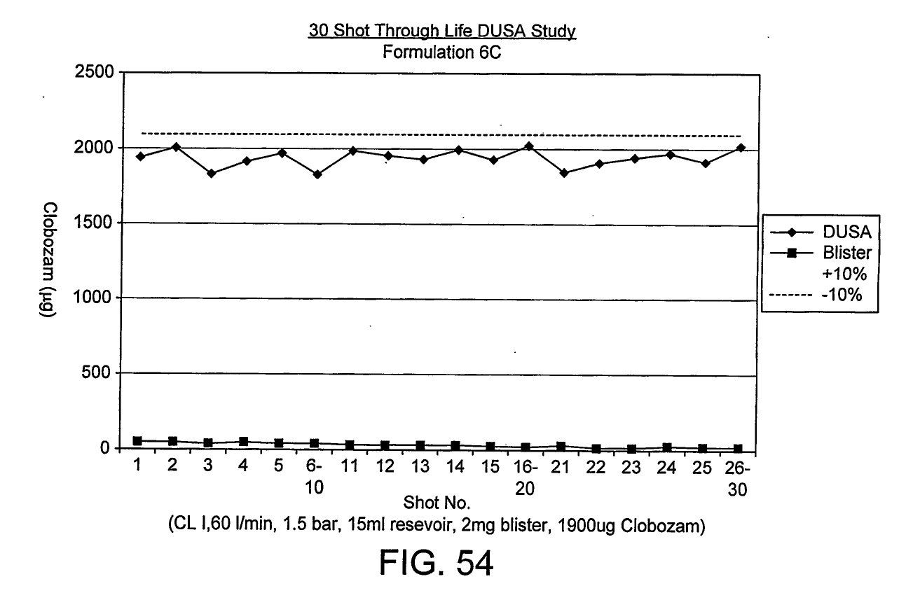

The reproducibility of the FPFs obtained was also tested. Through life dose uniformity for the primary candidate, 6C, the preparation of which is described above, was tested by firing 30 doses, with the emitted doses collected by DUSA. Through life dose uniformity results are presented in the graph of FIG. 54.

The mean ED was 1965 μg, with an RSD (relative standard deviation) of 2.8%. This material consequently demonstrated excellent through life dose reproducibility.

The results of particle size testing by Malvern of these powdered materials are provided in the following figures. The particle size distributions indicate the level of size reduction obtained by the co-milling.

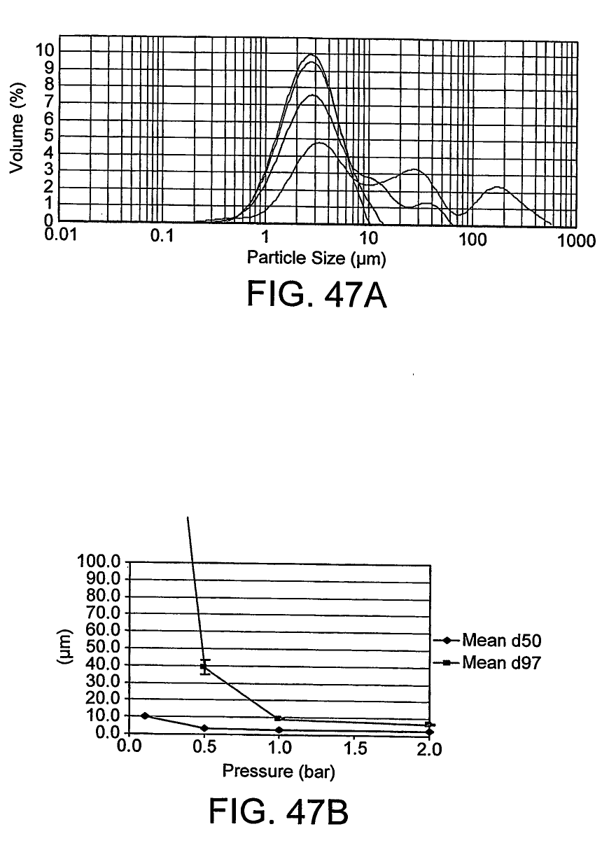

The results of dispersion testing of these powdered materials are provided in the FIGS. 47A to 53B. The particle size distributions indicate both the level of size reduction obtained by the co-milling, and the level of dispersion efficiency at varied pressures. The d50 and d97 plots provide a further indication of this dispersibility of the powders as a function of pressure.

The graphs in FIGS. 47A to 53A figures show the particle size distribution, with the four curves representing powder jet-milled at different pressures, namely at 2.0 bar, 1.0 bar, 0.5 bar and at 0.1 bar. The graphs in FIG. 47B to 53B show the level of dispersion efficiency at different pressures, in terms of d50 and d97.

FIGS. 47A and 47B are the results of testing formulation “3A”;

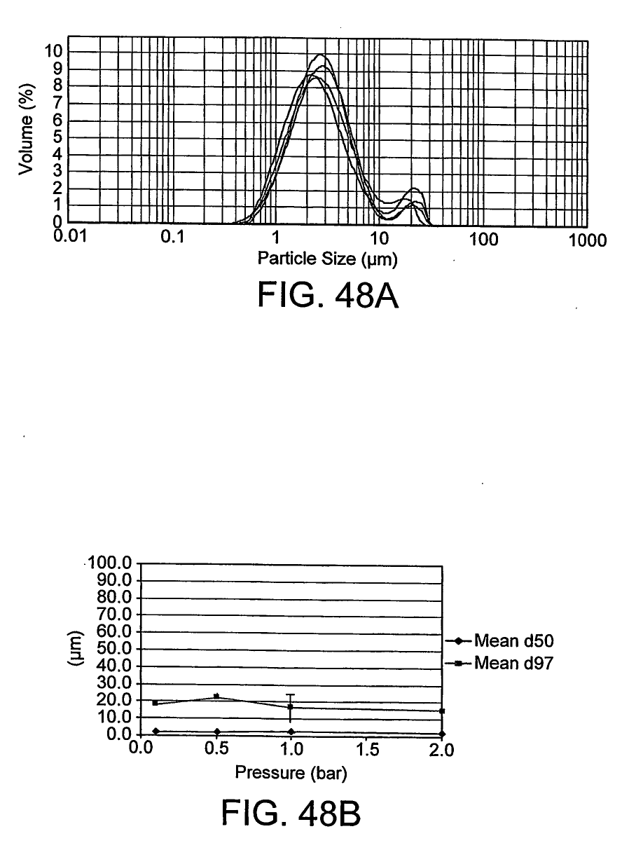

FIGS. 48A and 48B are the results of testing formulation “4B”;

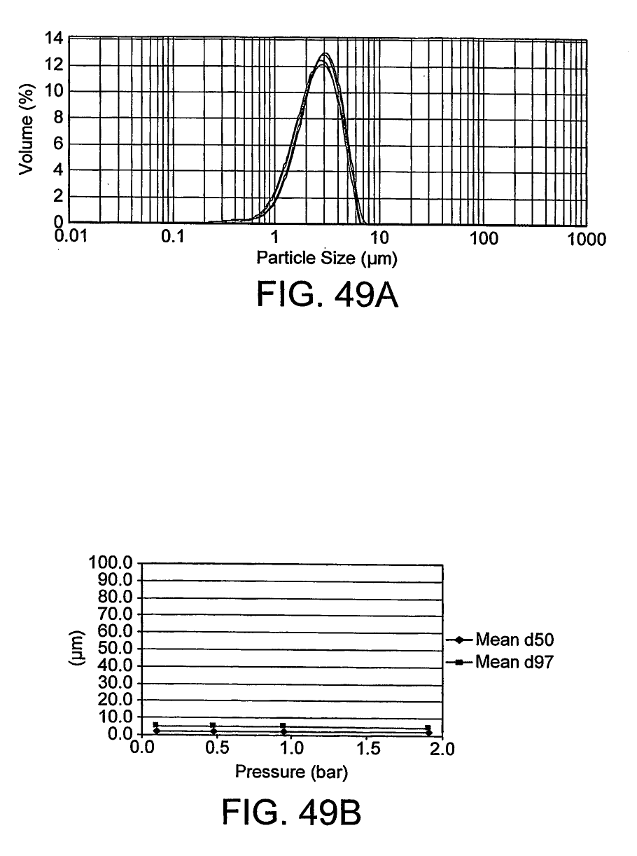

FIGS. 49A and 49B are the results of testing formulation “5B”;

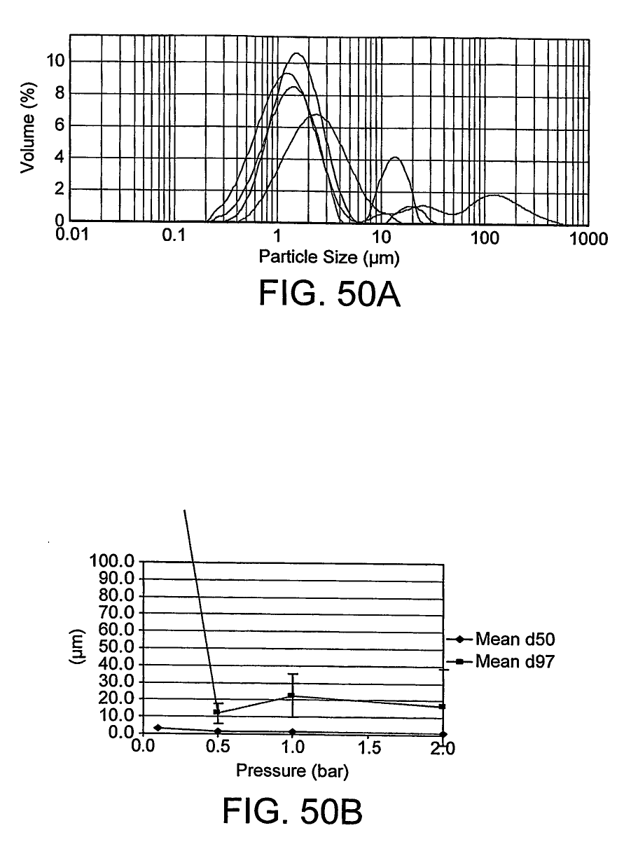

FIGS. 50A and 50B are the results of testing formulation “6C”;

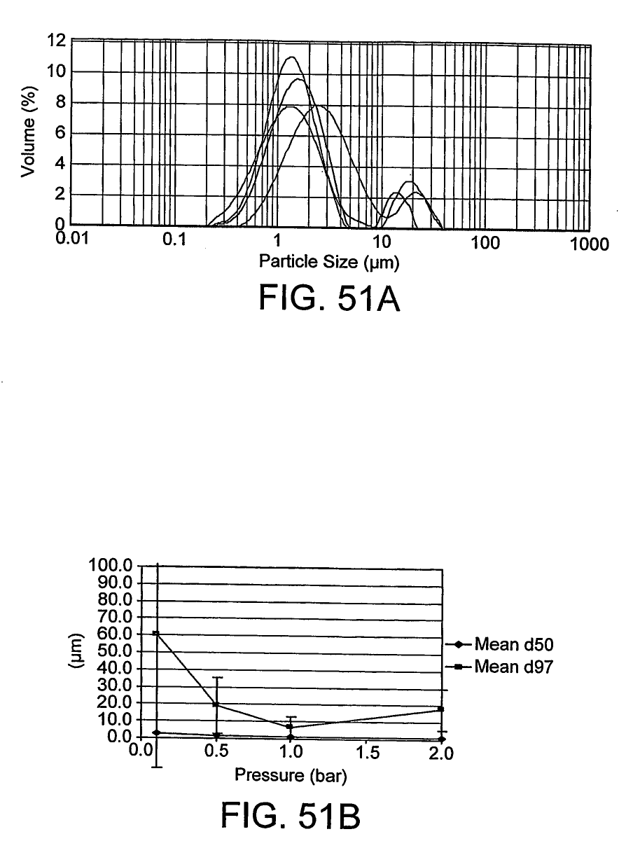

FIGS. 51A and 51B are the results of testing formulation “7C”;

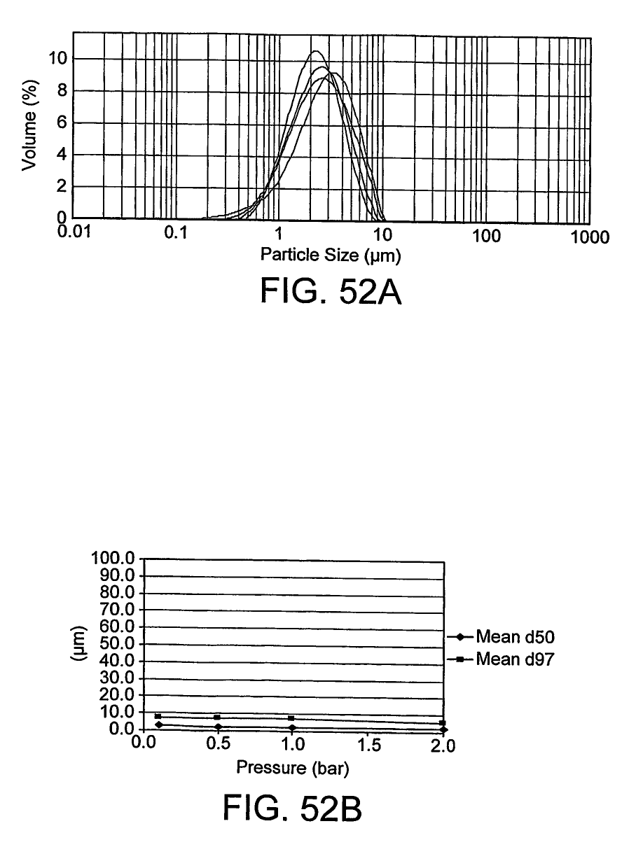

FIGS. 52A and 52B are the results of testing formulation “8C”; and

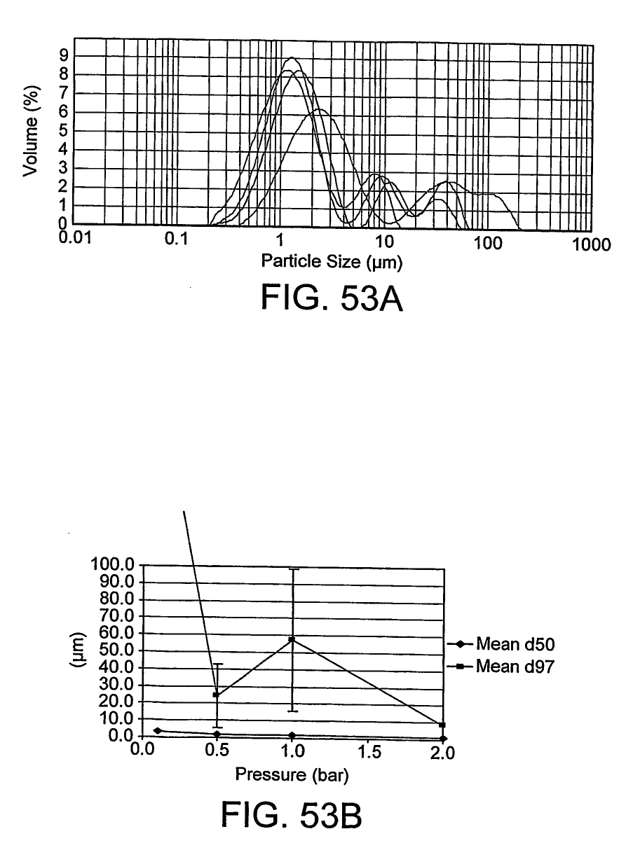

FIGS. 53A and 53B are the results of testing formulation “9B”.

From the graphs, one can see that formulation 5B exhibited much the best dispersion.

This set of dispersibility tests shows that the MechanoFused powders disperse more easily at lower pressures than the original drug, and that magnesium stearate gives the best dispersion within these, followed by Aerocine and leucine. The co-jet milled powders do not appear to disperse any more easily in this test than the original drug, however the primary particle sizes (d50) are reduced.

Comparison of Co-Jet Milled and Mechano-Fused Formulations (Apomorphine)

In order to establish the effect of co-jet milling on different active agents, apomorphine hydrochloride formulations with fine carrier particles (i.e. a 3-component system) were prepared and tested.

19.0 g of Sorbolac 400 lactose and 1.0 g of micronised L-leucine were combined in the Mechano-Fusion system. The material was processed at a setting of 20% power for 5 minutes, followed by a setting of 80% power for 10 minutes. This material was recovered and recorded as “2A”.

15.0 g of apomorphine hydrochloride and 0.75 g of micronised L-leucine were combined in the Mechano-Fusion system. The material was processed at a setting of 20% power for 5 minutes, followed by a setting of 80% power for 10 minutes. This material was recovered and recorded as “2B”.

2.1 g “2B” plus 0.4 g micronised leucine were blended by hand in a mortar and pestle for 2 minutes. 2.5 g micronised lactose was added and blended for a further 2 minutes. 5 g micronised lactose was added and blended for another 2 minutes. This mixture was then processed in the AS50 Spiral jet mill using an inlet pressure of 7 bar and a grinding pressure of 5 bar, feed rate 5 ml/min. This powder was gently pushed through a 300 μm metal sieve with a spatula. This material was recorded as “10A”.

1.5 g “110A” was combined with 0.20 g micronised L-leucine and 3.75 g of Sorbolac 400 lactose by hand in a mortar with a spatula for 10 minutes. This powder was gently pushed through a 300 μm metal sieve with a spatula. This material was recorded as “10B”.

9 g micronised apomorphine HCI plus 1 g micronised leucine were placed in the Mechano-Fusion system and processed at 20% (1000 rpm) for 5 minutes. This initial blend was then processed in the AS50 Spiral jet mill using an inlet pressure of 7 bar and a grinding pressure of 5 bar, feed rate 5 ml/min. This material was recorded as “11A”.

After blending, this powder was rested overnight, and then was gently passed through a 300 μm metal sieve by shaking. This material was recorded as “11B”.

2 g micronised apomorphine HCl plus 0.5 g micronised leucine were blended by hand in mortar and pestle for 2 minutes. 2.5 g micronised lactose was added and blended for a further 2 minutes. Then 5 g micronised lactose was added and blended for another 2 minutes. This mixture was then processed in the AS50 Spiral jet mill using an inlet pressure of 7 bar and a grinding pressure of 5 bar, feed rate 5 ml/min. This powder was gently pushed through a 300 μm metal sieve with a spatula. This material was recorded as “12A”.

16.5 g of Sorbolac 400 and 0.85 g of micronised leucine were placed in the Mechano-Fusion system and processed at 20% (1000 rpm) for 5 minutes then at 80% (4000 rpm) for 10 minutes. This material was recorded as “13A”.

0.5 g micronised apomorphine HCl plus 2.0 g “13A” were blended by hand in a mortar with a spatula for 10 minutes. This powder was gently pushed through a 300 μm metal sieve with a spatula. This material was recorded as “13B”.

A number of foil blisters were filled with approximately 2 mg of the following formulations:

10A—20% apomorphine HCl, 5% 1-leucine, 75% micronised lactose (co-jet milled)

10C—26.2% apomorphine HCl, 5% 1-leucine, 68.7% sorbolac (geometric)

11B—95% apomorphine HCl, 5% 1-leucine (co-jet milled)

12A—20% apomorphine HCl, 5% leucine, 75% micronised lactose (all co-jet milled)

13B—20% apomorphine HCl, 5% 1-leucine, 75% Sorbolac 400 (leucine & Sorbolac Mechano-Fused)

These were then fired from an Aspirair device into an NGI at a flow rate of 60 l/m. The Aspirair was operated with a reservoir of 15 ml at 1.5 bar. Each in vitro test was conducted once to screen, and then the selected candidates were repeated.

Further candidates were also repeated in ACI at 60 l/m.

TABLE 32

| Formulation | | | | |
|---|---|---|---|---|
| 2 mg, 1.5 bar | | | FPD | |
| 15 ml reservoir | | | (<5 μm) | |
| 60 l/min | MD (μg) | DD (μg) | (μg) | MMAD |
| 10A | 384 | 356 | 329 | 1.78 |
| 13B | 359 (1793) | 327 (1635) | 200 (1000) | 1.54 |
| 10C | 523 | 492 | 374 | 1.63 |
| 11B | 1891 | 1680 | 1614 | 1.36 |
| | 1882 | 1622 | 1551 | 1.44 |
| | 1941 | 1669 | 1601 | 1.49 |
| Ave. | 1905 | 1657 | 1589 | 1.43 |
| SD | 32 | 31 | 33 | 0.07 |
| RSD | 1.7 | 1.9 | 2.1 | 4.6 |
| 11B | 1895 | 1559 | 1514 | 1.58 |
| | 1895 | 1549 | 1485 | 1.62 |
| | 1923 | 1565 | 1504 | 1.62 |
| ACI | | | | |
| Ave. | 1904 | 1558 | 1501 | 1.61 |
| SD | 16 | 8 | 15 | 0.02 |
| RSD | 1 | 1 | 1 | 1 |
| 12A | 414 | 387 | 363 | 1.63 |
| | 410 | 387 | 363 | 1.66 |
| | 406 | 378 | 355 | 1.68 |
| Ave. | 410 | 384 | 360 | 1.66 |
| SD | 4 | 5 | 5 | 0.03 |
| RSD | 1 | 1 | 1 | 2 |
| Total ave. | 2050 | 1920 | 1800 | |
| 12A | 395 | 365 | 341 | 1.80 |
| | 411 | 385 | 360 | 1.85 |
| | 400 | 370 | 349 | 1.84 |
| ACI | | | | |
| Ave. | 402 | 373 | 350 | 1.83 |
| SD | 8 | 10 | 10 | 0.04 |
| RSD | 2 | 3 | 3 | 2 |
| Total ave. | 2011 | 1866 | 1750 | |

TABLE 33

| Formulation | | | | | |
|---|---|---|---|---|---|
| 2 mg, | | | | | |
| 1.5 bar | | | | | |
| 15 ml | FPF(MD) | FPF(ED) | FPF(ED) | FPF(ED) | FPF(ED) |
| reservoir | % | % | % | % | % |
| 60 l/min | (<5 um) | (<5 um) | (<3 um) | (<2 um) | (<1 um) |
| 10A | 86 | 93 | 87 | 60 | 13 |
| 13B | 56 | 61 | 52 | 42 | 19 |
| 10C | 72 | 76 | 67 | 51 | 16 |
| 11B | 85 | 96 | 95 | 81 | 24 |
| | 82 | 96 | 93 | 77 | 22 |
| | 82 | 96 | 92 | 74 | 20 |
| Ave. | 83 | 96 | 93 | 77 | 22 |
| SD | | 0 | 1.5 | 3.5 | 2 |
| RSD | | 0 | 1.6 | 4.5 | 9.1 |
| 11B | 80 | 97 | 94 | 74 | 14 |
| | 78 | 96 | 93 | 70 | 14 |
| | 78 | 96 | 94 | 72 | 12 |
| ACI | | | | | |
| Ave. | 79 | 96 | 94 | 72 | 13 |
| SD | | 1 | 1 | 2 | 1 |
| RSD | | 1 | 1 | 3 | 9 |
| 12A | 88 | 94 | 89 | 68 | 13 |
| | 89 | 94 | 89 | 66 | 12 |
| | 87 | 94 | 88 | 64 | 12 |
| Ave. | 88 | 94 | 8.9 | 66 | 12 |
| SD | | 0 | 1 | 2 | 1 |
| RSD | | 0 | 1 | 3 | 5 |
| 12A | 86 | 94 | 85 | 57 | 9 |
| | 88 | 93 | 84 | 55 | 8 |
| | 87 | 94 | 85 | 56 | 8 |
| ACI | | | | | |
| Ave. | 87 | 94 | 85 | 56 | 8 |
| SD | | 1 | 1 | 1 | 1 |
| RSD | | 1 | 1 | 2 | 7 |

TABLE 34

| Formulation | | | | |
|---|---|---|---|---|
| 2 mg, 1.5 bar | | | | |
| 15 ml reservoir | | | | |
| 60 l/min | Recovery | Throat | Blister | Device |
| 10A | 96% | 5% | 0.3% | 7% |
| 13B | 94% | 29% | 3% | 6% |
| 10C | 100% | 16% | 2% | 4% |
| 11B | 101% | 2% | 0.6% | 10% |
| | 99% | 2% | 0.2% | 14% |
| | 102% | 2% | 0.3% | 14% |
| Ave. | 101% | 2% | 0.4% | 13% |
| SD | 1.5 | 0 | 0.2 | 2.3 |
| RSD | 1.5 | 0 | 57 | 18 |
| 11B | 100% | 1% | 0.5% | 17% |
| | 100% | 2% | 0.1% | 18% |
| | 101% | 2% | 0.4% | 18% |
| ACI | | | | |
| Ave. | 100% | 2% | 0.3% | 18% |
| SD | 1 | 1 | 0.2 | 1 |
| RSD | 1 | 35 | 62 | 3 |
| 12A | 109% | 4% | 0.3% | 6% |
| | 108% | 4% | 0.2% | 6% |
| | 107% | 4% | 0.02% | 7% |
| Ave. | 108 | 4% | 0.2 | 6% |
| SD | 1 | 0 | 0.1 | 1 |
| RSD | 1 | 0 | 82 | 9 |
| 12A | 104% | 3% | 0.4% | 7% |
| | 108% | 4% | 0.2% | 6% |
| | 105% | 2% | 0.4% | 7% |
| ACI | | | | |
| Ave. | 106% | 3% | 0.3 | 7% |
| SD | 2 | 1 | 0.1 | 1 |
| RSD | 2 | 33 | 35 | 9 |

The co-jet milled formulations once again exhibited exceptional FPFs when it is dispensed using an active dry powder inhaler device. The improvement appears to be largely due to reduced throat deposition which was less than 5%, compared to between 16 and 29% for the Mechano-Fused formulations. “12A” was produced as a repeat of “10A”, but excluding the Mechano-Fused pre-blend (to show it was not required).

The reproducibility of the FPPs obtained with the formulation 12A, the preparation of which is described above, was tested.

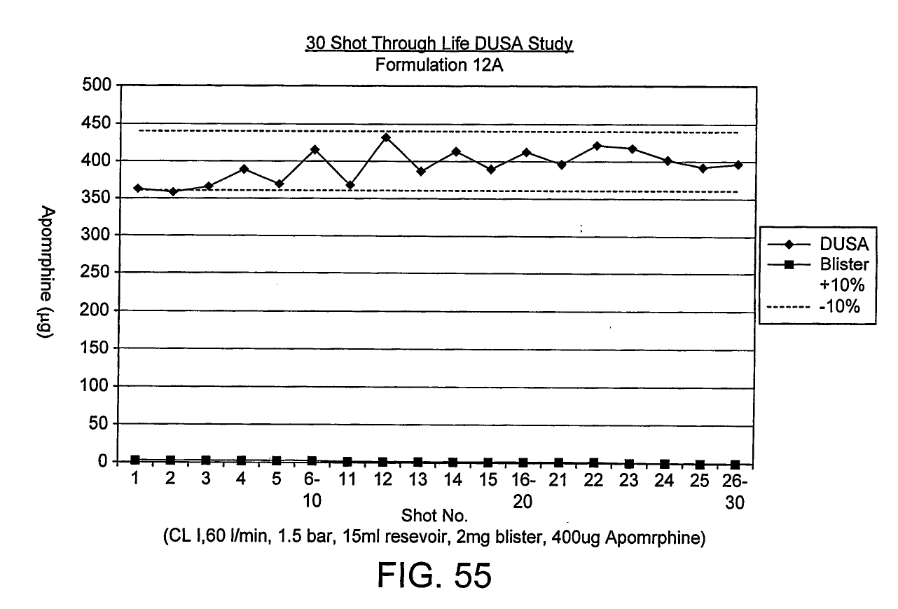

A number of foil blisters were filled with approximately 2 mg of formulation 12A. Through life dose uniformity was tested by firing 30 doses, with the emitted doses collected by DUSA. Through life dose uniformity results are presented in the graph in FIG. 55.

The mean ED was 389 μg, with an RSD of 6.1% and the through life delivery of this drug-lactose formulation was very good.

In order to investigate the cause of the unexpected differences between the co-jet milled formulations and those prepared by Mechano-Fusion, formulations “11B”, “10A” and “2C” were fired from an Aspirair and plume and vortex behaviour recorded on digital video. The images were studied in light of the above differences in throat deposition.

Video of plume behaviour indicated a difference between the co-jet milled formulations and Mechano-Fused formulations. Mechano-Fused formulations showed a highly concentrated fast moving bolus at the front of the air jet. Most powder appeared to have been emitted after approximately 40 ms. Co-jet milled formulations showed a greater spread of the plume. The plume front moves at a similar velocity, but the front is less concentrated, appears to slow more quickly and powder is emitted for considerably longer (i.e. >200 ms).

Video of the vortex showed that the Mechano-Fused powders enter the vortex within 10 ms, whereas co-jet milled formulations take at least 30 ms. Similarly the Mechano-Fused powders appeared quicker to leave the vortex, with the co-jet milled materials forming a more prolonged fogging of the vortex. The behaviour observed for co-jet milled materials was described as an increased tendency to stick, but then scout from the inside of the vortex.

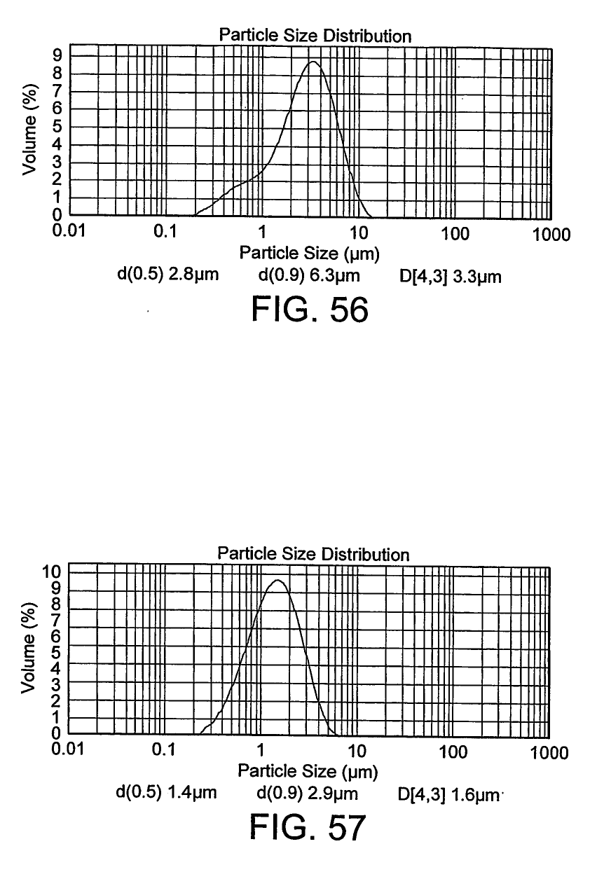

Particle size distributions of the raw materials and selected formulations were determined by Malvern particle sizer, via the Scirroco dry powder disperser. The data are summarised in the graphs shown in FIGS. 56 to 63.

FIG. 56 shows the particle size distribution of the raw material micronised lactose (833704);

FIG. 57 shows the particle size distribution of the raw material apomorphine;

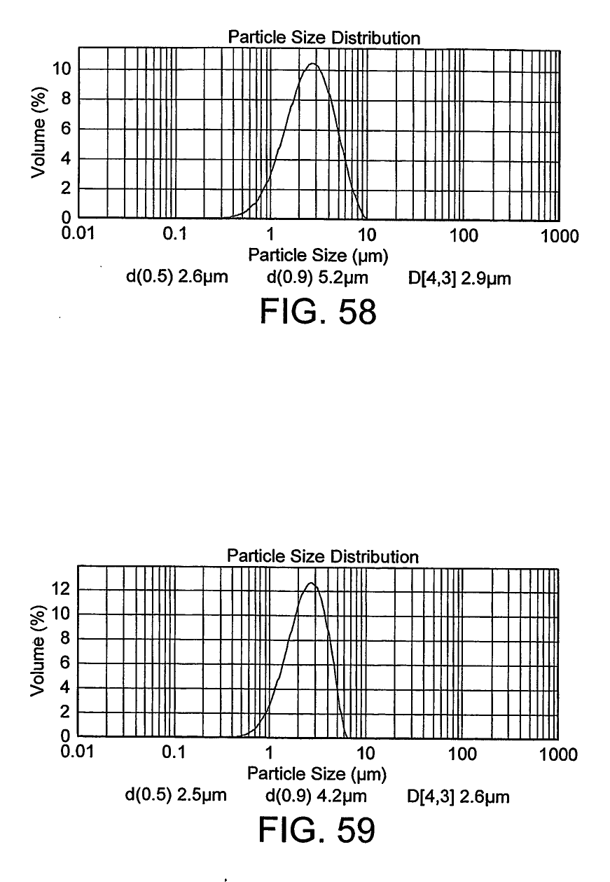

FIG. 58 shows the particle size distribution of the raw material clobozam;

FIG. 59 shows the particle size distribution of the clobozam formulation comprising 95% clobozam and 5% Mechano-Fused magnesium stearate;

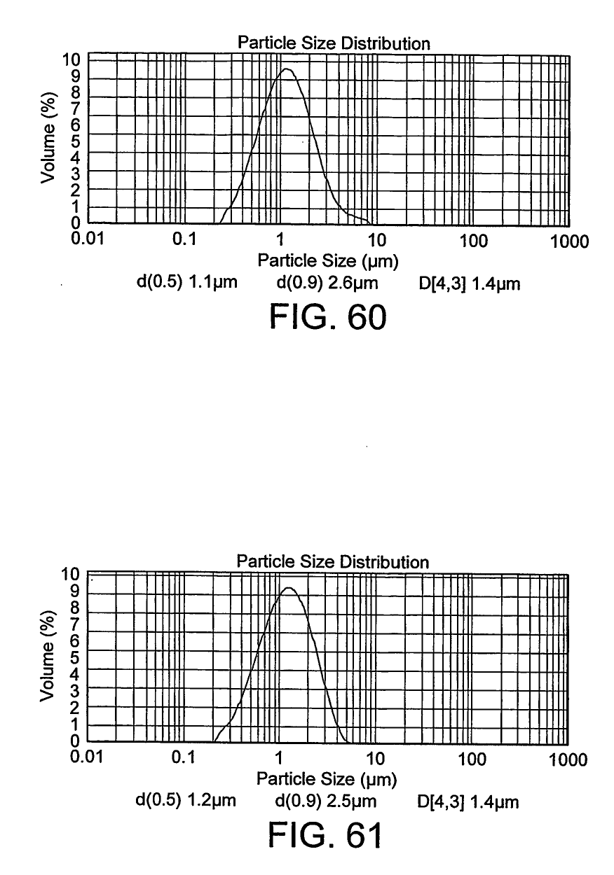

FIG. 60 shows the particle size distribution of the clobozam formulation comprising 95% clobozam and 5% co-jet milled Aerocine;

FIG. 61 shows the particle size distribution of the clobozam formulation comprising 95% clobozam and 5% co-jet milled leucine;

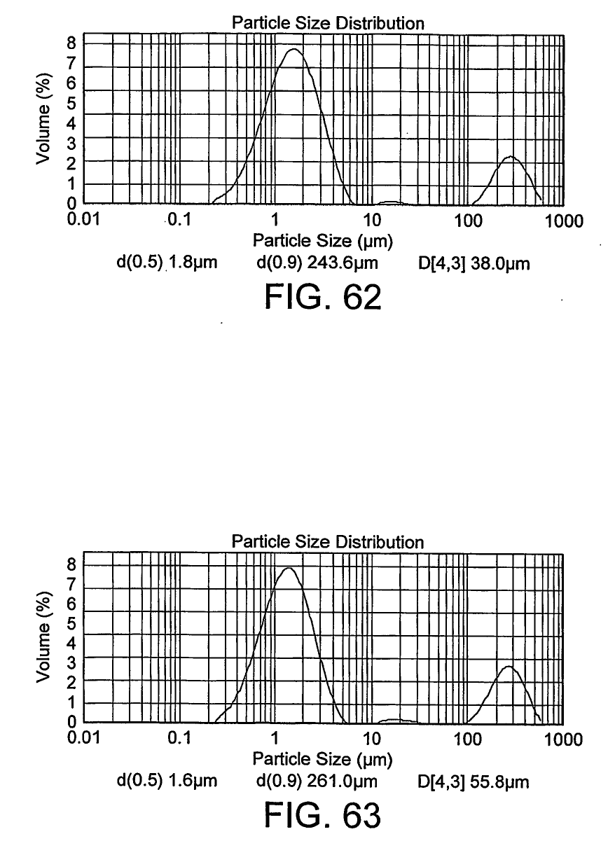

FIG. 62 shows the particle size distribution of the apomorphine formulation comprising 75% lactose, 20% apomorphine and 5% co-jet milled leucine; and

FIG. 63 also shows the particle size distribution of the apomorphine formulation comprising 75% lactose, 20% apomorphine and 5% co-jet milled leucine.

Where clobozam is co-jet milled with an additive material, a significant drop in particle size is observed. This is not seen for the clobozam Mechano-Fused formulation here.

With the apomorphine-lactose co-milled materials, the size distribution is low (d₅₀ 1.8 to 1.6), when compared to the particle size distribution of the micronised lactose which comprises 75% of the composition. However, size reduction is not detectable with respect to pure apomorphine, although it should be noted that this comprises only 20% of the powder composition.

In vitro data confirm that, surprisingly, Mechano-Fusion of active particles increased the throat deposition substantially. Mechano-Fusion has previously been associated with improvement in dispersibility from a passive device, and reduced throat deposition. In this case, Mechano-Fusion with magnesium stearate gives slightly lower throat deposition than Mechano-Fusion with leucine.

The throat deposition appears especially high for Mechano-Fused formulations containing leucine. It is speculated that this could be due to an agglomerating affect during Mechano-Fusion specific to leucine and not magnesium stearate, or an electrostatic effect specific to leucine.

However, surprisingly co-jet milling produces materials which, in comparison, give very low throat deposition, low device deposition and excellent dispersion from an active device. This co-jet milling also produces a significant further size reduction, for example, d50 changes from about 2.6 μm to about 1 μm for clobozam. When these factors are combined, a remarkable aerosolisation performance is obtained from the in-vitro tests. FPF(ED) are 90 to 96%. This excellent performance was obtained for leucine, Aerocine and magnesium stearate, and for 3 different formulations, including 2 different active agents, with or without lactose diluent.

The consequence of this is the achievement of a very low oropharangeal deposition to the patient, typically of approximately 5%. Given that both throat and upper airway deposition (corresponding to impactor throat and upper impactor stages) is reduced to a minimum, this will also result in a minimised tasteable component, and minimised fraction delivered to the GI tract. This corresponds to a 4-fold reduction in comparison to formulations without additive material.

It was noted that the co-jet milled materials were highly agglomerated in appearance, in contrast to the Mechano-Fused blends, which appeared as more free flowing powders.

Studies suggest that the difference between the performance of the co-jet milled and Mechano-Fused compositions is most apparent when the formulations are dispensed using an active device, such as Aspirair. Video of plume behaviour provided some indication of the reason for differences between the co-jet milled formulations and Mechano-Fused formulations. Mechano-Fused formulations showed a short fast bolus, whereas co-jet milled formulations showed a more drawn out plume. The “enhanced” flow properties of the Mechano-Fused powders appear to explain their worse Aspirair performance. A degree of powder hold-up within the device appears to be beneficial, allowing a less dense and extended plume to occur.

These video observations suggest the throat deposition difference is related to the powder lifetime within the vortex, with a longer lifetime giving reduced throat deposition. Lower aerosol concentration at the plume front, lower momentum of aerosol plume (with lower cloud density and smaller particle size) and greater opportunity to be de-agglomerated are possible contributors to this improvement. Also, there is also more material in the later, slower part of the plume. Furthermore, lower powder density in the cyclone appears to lead to better dispersion.

It is speculated that the fact that the powder formulations are difficult to extract from the blister actually enhances their delivery characteristics. It slows the extraction of the powder and so the active particles are travelling slower when they are expelled from the dispensing device. This means that the active particles do not travel at the front of the plume of powder created when the device is actuated and this means that the active particles are significantly less likely to impact on the throat of the user. Rather, the active particles are thought to be further back in the plume, which allows them to be inhaled and administered to the lung. Naturally, too much blister retention will be undesirable, as it will result in active agent remaining in the device after actuation.

In general, the co-milling of active particles with additive particles has yielded reduced device/blister retention compared to formulations prepared without additive particles. Mechano-Fusion was shown to give significantly greater blister retention than co-jet milling. The worst blister retention was seen for Mechano-Fused clobozam with magnesium stearate (13%). This appears related to the dusting nature of such formulations. The Mechano-Fused powders spread and flow more easily, which facilitates higher degrees of contact with the surfaces in bulk powder contact. The co-milled powders however are heavily agglomerated, so contact with surfaces is much reduced, and dust residues are also much less. The device retention also appears greater for Mechano-Fused than co-jet milled powders for clobozam. However, the device retention of apomorphine HCl co-jet milled with leucine appears notably high, at 13%. Device and blister retention does not appear substantially different between the 0.5 and 1.5 bar tests, except for the case of the unaltered pure clobozam, where device retention approaches 50% for the 0.5 bar test.

The tendency of a powder formulation to stick in the blister can be overcome in active devices, where a significant amount of turbulence is created within the blister when the device is actuated. However, this is not the case in a passive device. Therefore, the tendency of a formulation to stick in the blister will have a detrimental effect on the performance of a powder administered using a passive device. That said, as the active particles in the powder dispensed by a passive device are generally not moving as quickly as they would if dispensed by an active device, the problem of throat deposition (usually a result of the active particles travelling at the front of the powder plume) is not so great. Thus, it is clear that the properties of the active particles need to be tailored to the type of device used to dispense the powder.

Tests were carried out to compare the FPF achieved when the co-jet milled compositions are dispensed using passive and active devices. The experiments used a lactose model fixed into a TSI. The results were as follows:

TABLE 35

| | FPF(ED) | FPF(MD) % | FPF(MD) % |
|---|---|---|---|
| Formulation | % | (Cyclohaler) | (Aspirair) |
| Micronised lactose | 32 | 18 | — |
| With 5% magnesium stearate | 35 | 32 | 27 |
| (MgSt) in a conventional blender | | | |
| 5% MgSt jet-milled at 2 bar | 68 | 53 | 62 |
| 5% MgSt jet-milled at 7 bar | 52 | 39 | 72 |
| 5% MgSt Mechano-Fused | 69 | 57 | 49 |

This shows that jet milled material which has been co-jet milled at low pressure is better in passive devices whilst high pressure jet milled materials perform better in active devices such as Aspirair.

Mechano-Fused Budesonide with Magnesium Stearate

The magnesium stearate selected was a standard grade supplied by Avocado Research Chemicals Ltd., Lot H1028A. The drug used was budesonide.

This work was conducted using the Miat Monohaler. The work studied systems of magnesium stearate processed with budesonide. The blends were prepared by mechano-fusion using the Hosakawa AMS-MINI, blending for 60 minutes at approximately 4000 rpm.

Blends of budesonide and magnesium stearate were prepared at different weight percentages of magnesium stearate. Blends of 5% w/w and 10% w/w were prepared and then tested. MSLIs and TSIs were carried out on the blends. The results, which are summarised below, indicate a high aerosolisation efficiency.

| | | FPD | ED | |
|---|---|---|---|---|
| Formulation | FPF(ED) | mg | mg | Method |
| Budesonide:MgSt | 73% | 1.32 | 1.84 | MSLI |
| (5% w/w) | | | | |
| Budesonide:MgSt | 80% | 1.30 | 1.63 | TSI |
| (10% w/w) | | | | |

Mechano-Fused Budesonide with Fine Lactose and Magnesium Stearate

A further study was conducted to look at the Mechano-Fusion of a drug with both a force control agent and a fine lactose. The force control agent used was magnesium stearate (Avocado) and the fine lactose was Sorbolac 400 (Meggle). The drug used was budesonide (2M00M0-0019427). The blends were prepared by Mechano-Fusion using the Hosakawa AMS-MINI, blending for 60 minutes at approximately 4000 rpm.

Formulations were prepared using the following concentrations of budesonide, magnesium stearate and Sorbolac 400:

5% w/w budesonide, 6% w/w MgSt, 89% w/w Sorbolac 400

20% w/w budesonide, 6% w/w MgSt, 74% w/w Sorbolac 400

TSIs and MSLIs were performed on the blends. The results summarised below indicate that, as the amount of budesonide in the blends increased, the FPF results also increased. Device and capsule retention were notably low in these dispersion tests (>5%).

| | FPF(ED) | FPF(ED) |
|---|---|---|
| Formulation | (TSI) | (MSLI) |
| 5:6:89 | 66.0% | 70.1% |
| 20:6:74 | 75.8% | — |

As an extension to this work, different blending methods of the budesonide, magnesium stearate and Sorbolac 400 were investigated further.

Two formulations were prepared in the Glen Creston Grindomix. This mixer is a conventional food-processor style bladed mixer, with 2 parallel blades.

The first of these formulations was a 5% w/w budesonide, 6% w/w MgSt, 89% w/w Sorbolac 400 blend prepared by mixing all components together at 200 rpm for 20 minutes. The formulation was tested by TSI and the results, when compared to those for the mechano-fused blends, showed the Grindomix blend to give lower FPF results (see table below).

The second formulation was a blend of 90% w/w of mechanofused magnesium stearate:Sorbolac 400 (5:95) pre-blend and 10% w/w budesonide blended in the Grindomix for 20 minutes. The formulation was tested by TSI and MSLI.

It was also observed that this formulation had notably good flow properties for a material comprising such fine particles: this was associated with the Mechano-Fusion process.

| | FPF | FPF |
|---|---|---|
| Formulation | (TSI) | (MSLI) |
| Grindomix 5:6:89 | 57.7% | — |
| Grindomix 10% budesonide | 65.9% | 69.1% |
| (mechanofused pre-blend) | | |

Mechano-Fused Salbutamol with Fine Lactose and Magnesium Stearate

A further study was conducted to look at the Mechano-Fusion of a further drug with both a force control agent and the fine lactose. The force control agent used was magnesium stearate and the fine lactose was Sorbolac 400 (Meggle). The drug used was micronised salbutamol sulphate. The blends were prepared by Mechano-Fusion using the Hosakawa AMS-MINI, blending for 10 minutes at approximately 4000 rpm.

The formulations prepared were:

20% w/w salbutamol, 5% w/w MgSt, 75% w/w Sorbolac 400

20% w/w salbutamol, 2% w/w MgSt, 78% w/w Sorbolac 400

NGIs were performed on the blends and the results are set out below. Device and capsule retention were again low in these dispersion tests (>10%).

| Formulation | FPF(ED) | FPF(ED) |
|---|---|---|
| 20:5:75 | 80% | 74% |
| 20:2:78 | 78% | 70% |

Co-Jet Milled Clomipramine hydrochloride Formulations in Aspirair

Clomipramine hydrochloride was obtained in powdered form. Force control agents leucine and magnesium stearate were used.

Twelve formulations were produced from the original powder, using the Hosokawa AS50 jet mill. Either the pure drug was passed through the mill or a blend of drug with 5% w/w of a force control agent added. The mill was used with a range of parameters. Primarily, these were injector air pressure, grinding air pressure and powder feed rate.

Formulation 14: The pure clomipramine hydrochloride was passed through the microniser three times, each time with an injector air pressure of 8 bar, grinding air pressure of 1.5 bar and powder feed rate of ˜1 g/min. Malvern (dry powder) particle size measurement gave a d(50) of 1.2 μm.

Formulation 15: Formulation 14 was pre-blended in a pestle with a spatula with 5% micronised 1-leucine. This blend was further micronised with an injector air pressure of 8 bar, grinding air pressure of 1.5 bar and powder feed rate of ˜1 g/min. Malvern (dry powder) particle size measurement gave a d(50) of 1.2 μm.

Formulation 16: The pure clomipramine hydrochloride was micronised with an injector air pressure of 7 bar, grinding air pressure of 5 bar and powder feed rate of ˜10 g/min. Malvern (dry powder) particle size measurement gave a d(50) of 1.0 μm.

Formulation 17: The pure clomipramine hydrochloride was micronised with an injector air pressure of 7 bar, grinding air pressure of 5 bar and powder feed rate of ˜10 g/min. This micronised clomipramine hydrochloride was pre-blended in a pestle with a spatula with 5% micronised 1-leucine. This blend was then micronised with an injector air pressure of 7 bar, grinding air pressure of 5 bar and powder feed rate of 10 g/min. Malvern (dry powder) particle size measurement gave a d(50) of 0.95 μm.

Formulation 18: The pure clomipramine hydrochloride was pre-blended in a pestle with a spatula with 5% magnesium stearate. This blend was micronised with an injector air pressure of 7 bar, grinding air pressure of 5 bar and powder feed rate of ˜10 g/min. Malvern (dry powder) particle size measurement gave a d(50) of 0.95 μm.

Formulation 19: The pure clomipramine hydrochloride was micronised with an injector air pressure of 7 bar, grinding air pressure of 1 bar and powder feed rate of ˜1 g/min. Malvern (dry powder) particle size measurement gave a d(50) of 1.8 μm.

This pre-micronised clomipramine hydrochloride was then blended in a pestle with a spatula with 5% micronised 1-leucine. This blend was then micronised with an injector air pressure of 7 bar, grinding air pressure of 1 bar and powder feed rate of ˜1 g/min. Malvern (dry powder) particle size measurement gave a d(50) of 1.38 μm.

Formulation 20: The pure clomipramine hydrochloride was micronised with an injector air pressure of 7 bar, grinding air pressure of 1 bar and powder feed rate of ˜10 g/min. Malvern (dry powder) particle size measurement gave a d(50) of 3.5 μm.

This pre-micronised clomipramine hydrochloride was then blended in a pestle with a spatula with 5% micronised 1-leucine. This blend was then micronised with an injector air pressure of 7 bar, grinding air pressure of 1 bar and powder feed rate of ˜10 g/min. Malvern (dry powder) particle size measurement gave a d(50) of 2.0 μm.

Formulation 21: The pure clomipramine hydrochloride was micronised with an injector air pressure of 7 bar, grinding air pressure of 3 bar and powder feed rate of ˜1 g/min. Malvern (dry powder) particle size measurement gave a d(50) of 1.2 μm.

This pre-micronised clomipramine hydrochloride was then blended in a pestle with a spatula with 5% micronised 1-leucine. This blend was then micronised with an injector air pressure of 7 bar, grinding air pressure of 3 bar and powder feed rate of ˜1 g/min. Malvern (dry powder) particle size measurement gave a d(50) of 0.99 μm.

Formulation 22 The pure clomipramine hydrochloride was micronised with an injector air pressure of 7 bar, grinding air pressure of 3 bar and powder feed rate of ˜10 g/min. Malvern (dry powder) particle size measurement gave a d(50) of 1.6 μm.

This pre-micronised clomipramine hydrochloride was then blended in a pestle with a spatula with 5% micronised 1-leucine. This blend was then micronised with an injector air pressure of 7 bar, grinding air pressure of 3 bar and powder feed rate of ˜10 g/min. Malvern (dry powder) particle size measurement gave a d(50) of 1.1 μm.

Formulation 23: The clomipramine hydrochloride was pre-blended in a pestle with a spatula with 5% micronised 1-leucine. This blend was micronised with an injector air pressure of 7 bar, grinding air pressure of 5 bar and powder feed rate of ˜10 g/min. Malvern (dry powder) particle size measurement gave a d(50) of 1.8 μm.

Formulation 24: The pure clomipramine hydrochloride was micronised with an injector air pressure of 7 bar, grinding air pressure of 5 bar and powder feed rate of ˜10 g/min.

This pre-micronised clomipramine hydrochloride was then blended in a pestle with a spatula with 5% magnesium stearate. This blend was then micronised with an injector air pressure of 7 bar, grinding air pressure of 1 bar and powder feed rate of ˜10 g/min. Malvern (dry powder) particle size measurement gave a d(50) of 1.38 μm.

Formulation 25: Formulation 24 was then processed in the Hosokawa MechanoFusion Minikit with 1 mm compression gap for 10 minutes. Malvern (dry powder) particle size measurement gave a d(50) of 1.391 μm.

Particle size distributions

The Malvern particle size distributions show that clomipramine hydrochloride micronised very readily to small particle sizes. For example, Formulation 16 micronised to 1.0 μm with one pass at the relatively high grinding pressure of 5 bar and the higher powder feed rate of 10 g/min.

Reducing the grinding pressure, for example to 1 bar as with Formulation 19 interim powder, resulted in larger particles (d(50)˜1.8 μm). Intermediate grinding pressure (3 bar) gave an intermediate particle size distribution (d(50) ˜1.2 μm as for Formulation 21 interim powder).

Similarly, increasing powder feed rate, for example from 1 to 10 g/min, resulted in larger particles, as can be seen by comparing d(50)s for Formulations 19 and 20.

The addition of an additive material, for example leucine as in Formulation 23, appeared to reduce the milling efficiency. However, this change may have been caused by the concomitant improvement in flowability of the original drug powder leading to a small but significant increase in the powder feed rate into the mill. It was observed in other studies that milling efficiency was increasingly sensitive to this powder feed rate as it increased above 10 g/mm.

It appeared possible from this series of examples to design the milling parameters to select a particular d(50). For example, a d(50) of ˜1.4 could be obtained either by repeated low pressure milling and low feed rate (Formulation 19) or by a mix of higher and lower pressure milling at a higher feed rate (Formulation 25).

Aspirair Dispersion Performance

Approximately 2 mg of each formulation was then loaded and sealed into a foil blister. This was then fired from an Aspirair device into a Next Generation Impactor with air flow set at 60 l/min. The performance data are summarised in Tables 36, 37 and 38.

TABLE 36

| | MD | DD | FPD | |
|---|---|---|---|---|
| Formulation | (mg) | (mg) | (mg) | MMAD |
| 14 | 1.64 | 1.19 | 1.05 | 1.53 |
| (pure drug, jet milled at 8/1.5 bar) | | | | |
| 15 | 1.55 | 1.32 | 1.19 | 1.68 |
| (5% leucine, jet-milled at 8/1.5 bar) | | | | |
| 16 | 2.414 | 1.832 | 1.493 | 1.80 |
| (pure drug, jet-milled at 7/5 bar) | | | | |
| 17 | 2.120 | 1.624 | 1.474 | 1.52 |
| (5% leucine, jet-milled at 7/5 bar) | | | | |
| 18 | 1.737 | 1.519 | 1.390 | 1.44 |
| (5% MgSt, jet-milled at 7/5 bar) | | | | |
| 19 | 2.031 | 1.839 | 1.550 | 1.90 |
| (5% leucine, jet-milled at 7/1 bar) | | | | |
| 20 | 1.821 | 1.685 | 1.071 | 2.44 |
| (5% leucine, jet-milled at 7/1 bar) | | | | |
| 21 | 1.846 | 1.523 | 1.437 | 1.61 |
| (5% leucine, jet-milled at 7/3 bar) | | | | |
| 22 | 2.213 | 1.940 | 1.733 | 1.72 |
| (5% leucine, jet-milled at 7/3 bar) | | | | |
| 23 | 1.696 | 1.557 | 1.147 | 2.13 |
| (5% leucine, single pass at 7/5 bar) | | | | |
| 24 | 1.743 | 1.542 | 1.274 | 1.82 |
| (5% MgSt, jet-milled at 7/5 bar & | | | | |
| Mechano-Fused) | | | | |
| 25 | 1.677 | 1.570 | 1.351 | 1.72 |
| (5% MgSt, jet-milled at 7/5 bar) | | | | |

TABLE 37

| | FPF % | FPF % | FPF % | FPF % |
|---|---|---|---|---|
| Formulation | (<5 μm) | (<3 μm) | (<2 μm) | (<1 μm) |
| 14 | 88 | 83 | 65 | 21 |
| (pure drug, jet milled at 8/1.5 bar) | | | | |
| 15 | 90 | 82 | 60 | 17 |
| (5% leucine, jet-milled at 8/1.5 | | | | |
| bar) | | | | |
| 16 | 82 | 71 | 51 | 14 |
| (pure drug, jet-milled at 7/5 bar) | | | | |
| 17 | 91 | 85 | 68 | 21 |
| (5% leucine, jet-milled at 7/5 bar) | | | | |
| 18 | 91 | 90 | 73 | 20 |
| (5% MgSt, jet-milled at 7/5 bar) | | | | |
| 19 | 84 | 74 | 48 | 10 |
| (5% leucine, jet-milled at 7/1 bar) | | | | |
| 20 | 64 | 46 | 28 | 6 |
| (5% leucine, jet-milled at 7/1 bar) | | | | |
| 21 | 94 | 88 | 67 | 14 |
| (5% leucine, jet-milled at 7/3 bar) | | | | |
| 22 | 89 | 80 | 56 | 14 |
| (5% leucine, jet-milled at 7/3 bar) | | | | |
| 23 | 74 | 57 | 37 | 9 |
| (5% leucine, single pass at 7/5 bar) | | | | |
| 24 | 83 | 68 | 47 | 15 |
| (5% MgSt, jet-milled at 7/5 bar & | | | | |
| Mechano-Fused) | | | | |
| 25 | 86 | 74 | 53 | 21 |
| (5% MgSt, jet-milled at 7/5 bar) | | | | |

TABLE 38

| | Re- | | | |
|---|---|---|---|---|
| | covery | Throat | Blister | Device |
| Formulation | % | % | % | % |
| 14 | 82 | 8 | 1 | 26 |
| (pure drug, jet milled at 8/1.5 bar) | | | | |
| 15 | 81 | 7 | 0 | 15 |
| (5% leucine, jet-milled at | | | | |
| 8/1.5 bar) | | | | |
| 16 | 121 | 10 | 3 | 21 |
| (pure drug, jet-milled at 7/5 bar) | | | | |
| 17 | 106 | 5 | 1 | 23 |
| (5% leucine, jet-milled at 7/5 bar) | | | | |
| 18 | 91 | 6 | 0 | 12 |
| (5% MgSt, jet-milled at 7/5 bar) | | | | |
| 19 | 107 | 10.6 | 1.3 | 8.2 |
| (5% leucine, jet-milled at 7/1 bar) | | | | |
| 20 | 96 | 24 | 1.3 | 6.1 |
| (5% leucine, jet-milled at 7/1 bar) | | | | |
| 21 | 97 | 3 | 0.6 | 16.9 |
| (5% leucine, jet-milled at 7/3 bar) | | | | |
| 22 | 116 | 7 | 0.6 | 16.9 |
| (5% leucine, jet-milled at 7/3 bar) | | | | |
| 23 | 87 | 18 | 2 | 6 |
| (5% leucine, single pass at 7/5 bar) | | | | |
| 24 | 92 | 14 | 1 | 10 |
| (5% MgSt, jet-milled at 7/5 bar & | | | | |
| Mechano-Fused) | | | | |
| 25 | 87 | 10 | 1 | 6 |
| (5% MgSt, jet-milled at 7/5 bar) | | | | |

The device retention appeared high (above 20%) where pure drug was used, and especially increased with small particle sizes (especially 1 μm and below): for example Formulations 14 and 16 had high drug retention. Device retention was lower with use of magnesium stearate, for example as with Formulation 18 where device retention was 12% despite a d(50) of 0.95 μm. Device retention was also reduced below 20% when leucine was used in combination with a particle size above 1 μm, for example with Formulation 22.

Throat deposition was reduced proportionately as particle size was reduced. High throat deposition (>20%) occurs with particle size d(50)>2 μm: e.g. Formulation 20. Throat deposition of below 10% was seen for particle sizes below 1 μm. The reduced inertial behaviour of the smaller particles may well contribute to this observation. However, as noted above, device retention tended to be greater for such small particles.

It is argued that as particle size was reduced, increased adhesiveness and cohesiveness results in increased device retention. This adhesiveness and cohesiveness and hence device retention can be reduced by addition of force control agents, attached to the drug particle surface (or drug and excipient particle surfaces, as appropriate). As argued previously for the apomorphine and clobozam examples, and demonstrated by the video study, in Aspirair it is believed that a level of adhesiveness and cohesiveness is desirable to prolong lifetime in the vortex, yielding a slower plume, but adhesiveness and cohesiveness should not be so high as to result in high device retention. Consequently a balance of particle size, adhesiveness and cohesiveness is required to achieve an optimum performance in Aspirair. The examples contained herein indicate how such a balance may be achieved. This balance may require modifying for each particular different material characteristic.

Single step co-milling with a force control agent appears effective in some examples such as Formulation 18. Multiple stage processing may be more effective for example, where the conditions are selected to achieve particularly desirable effects. For example, first stage high pressure milling of pure drug may be used to produce the required size distribution (i.e. ˜1.4 μm), and a second stage lower pressure co-milling used to mix in the force control agent, whereby better mixing is achieved without milling and with reduced segregation of components in the mill. Such is shown in Formulation 25, where a combination of both relatively low throat deposition and low device retention are achieved.

The optimum amount of additive material will depend on the chemical composition and other properties of the additive material and upon the nature of the active material and/or excipient material, if present. In general, the amount of additive material in the composite active particles will be not more than 60% by weight, based on the weight of the active material and any excipient material. However, it is thought that for most additive materials the amount of additive material should be in the range of 40% to 0.25%, preferably 30% to 0.5%, more preferably 20% to 2%, based on the total weight of the additive material and the active material being milled. In general, the amount of additive material is at least 0.01% by weight based on the weight of the active material.

Clearly, many different designs of jet mills exist and any of these may be used in the present invention. For example, in addition to the AS50 Spiral jet mill and the MC50 Hosakawa Micron used in the experiments discussed above, one can also use other spiral jet mills, pancake jet mills or opposed fluid bed jet mills. The feed rate for the jet mills will depend on their size. Small spiral jet mills might use a feed rate of, for example, 1 to 2 g per minute, whilst industrial scale mills will have a feed rate in the order of kilograms per hour.

The properties of the co-jet milled particles produced using the present invention may, to an extent, be tailored or adjusted by making changes to the jet milling apparatus. For example, the degree of particle coating and particle size reduction may be adjusted by changing the number of jets which are used in the apparatus, and/or by adjusting their orientation, that is, the angles at which they are positioned.

### CONCLUSIONS

The improvements in the dry powder inhaler devices and in the dry powder formulations mean that the desired dose efficiency can be achieved. The following tests demonstrate this.

The in-vitro testing was performed using the Aspirair device and using formulations prepared as follows.

120 g of Respitose SV003 lactose (45 to 63 μsieve fraction) and 30 g of micronised apomorphine hydrochloride and were combined into the mixing bowl of a Glen Creston GrindoMix high shear blender. The drug was sandwiched between Respitose layers. The material was processed at a setting of 2000 rpm for 5 minutes. The blend was screened through a 250 μm sieve.

Content uniformity was assessed by taking 10 samples of 3 mg from the bulk powder. The formulation contained a mean drug content of 20.8%, with a relative standard deviation of 1.97%.

2 mg of powder was filled into 25 Aspirair foil blisters. 5 blisters were fired from an Aspirair device into a 60 litre per minute Andersen Cascade Impactor (ACI), with air flow set at 60 litres per minute. The Aspirair was fired with 15 ml of reservoir air at 1.5 bar. This was repeated 5 times and the results are summarised in Tables 39 and 40.

TABLE 39

| | | | FPD | | |
|---|---|---|---|---|---|
| | MD | DD | (mg) | FPF(MD) % | FPF(ED) % |
| Formulation | (mg) | (mg) | (<5 μm) | (<5 μm) | (<5 μm) |
| | 0.38 | 0.36 | 0.29 | 75 | 81 |
| | 0.38 | 0.35 | 0.28 | 74 | 80 |
| | 0.40 | 0.37 | 0.30 | 75 | 81 |
| | 0.39 | 0.36 | 0.29 | 74 | 80 |
| | 0.38 | 0.35 | 0.29 | 75 | 82 |
| Mean | 0.39 | 0.36 | 0.29 | 75 | 81 |

TABLE 40

| | FPF(ED) | FPF(ED) | | Blister | Device |
|---|---|---|---|---|---|
| | % | % | | retention | retention |
| Formulation | (<3 μm) | (<2 μm) | MMAD | (%) | (%) |
| | 75 | 54 | 1.70 | 2 | 6 |
| | 74 | 52 | 1.73 | 2 | 6 |
| | 74 | 53 | 1.72 | 2 | 6 |
| | 74 | 55 | 1.66 | 2 | 6 |
| | 75 | 55 | 1.68 | 2 | 6 |
| Mean | 74 | 54 | 1.70 | 2% | 6% |

The formulations exhibited exceptional fine particle fractions of emitted dose and of metered dose. Also the performance is very consistent, between all 5 repeated tests.

In a further study, also using the CL1 Aspirair device, the following formulation was tested. Respitose SV003 lactose (45 to 63 μm sieve fraction) and micronised salbutamol sulphate were combined in the ratio 60:40.

1 mg of powder was filled into 15 Aspirair foil blisters. 5 blisters were fired from an Aspirair device into a Next Generation Impactor with air flow set at 60 litres per minute. The Aspirair was fired with 15 ml of reservoir air at 1.5 bar. This was repeated 3 times. The results are summarised in Tables 41 and 42.

TABLE 41

| | MD | ED | FPD >5 μm | FPF(MD) % | |
|---|---|---|---|---|---|
| NGI | (μg) | (μg) | (μg) | >5 μm | MMAD |
| 1 | 484 | 470 | 397 | 82 | 1.80 |
| 2 | 376 | 367 | 328 | 87 | 1.78 |
| 3 | 404 | 390 | 350 | 87 | 1.74 |

TABLE 42

| | FPF(ED) % | FPF(ED) % | FPF(ED) % | FPF(ED) % |
|---|---|---|---|---|
| NGI | >5 μm | >3 μm | >2 μm | >1 μm |
| 1 | 85 | 73 | 53 | 21 |
| 2 | 89 | 78 | 55 | 17 |
| 3 | 90 | 79 | 56 | 19 |

Once again, the formulations exhibited exceptional and reproducible fine particle fractions of emitted dose and of metered dose.

### EXAMPLE 1

### Inhalation Testing

The above referenced blisters containing the 100 and 200 microgram apomorphine-lactose formulations were subjected to testing using an Aspirair prototype inhaler.

In order to obtain the inhalation data described below, the inhaler device was used in conjunction with three instruments, a Multi-Stage Liquid Impinger (MSLI) (U.S.P. 26, Chapter 601, Apparatus 4 (2003), an Anderson Cascade Impactor (ACI) (U.S.P. 26, Chapter 601, Apparatus 3 (2003), and a Dosage Unit Sampling Apparatus (DUSA) (U.S.P. 26, Chapter 601, Apparatus B (2003). Each of these devices has an input for receiving the mouthpiece of the inhaler.

The DUSA is used to measure the total amount of drug which leaves the inhaler. With data from this device, the metered and delivered dose is obtained. The delivered dose is defined as the amount of drug that leaves the inhaler. This includes the amount of drug in the throat of the DUSA device, in the measuring section of the DUSA device and the subsequent filters of the DUSA device. It does not include drug left in the blister or other areas of the inhaler, and does not account for drug “lost” in the measuring process of the DUSA device. The metered dose includes all of the drug which leaves the blister.

The MSLI is a device for estimating deep lung delivery of a dry powder formulation. The MSLI includes a five stage cascade impactor which can be used for determining the particle size (aerodynamic size distribution) of Dry Powder Inhalers (DPIs) in accordance with USP 26, Chapter 601, Apparatus 4 (2003) and in accordance with the European Pharmacopoeia, Method 5.2.9.18, Apparatus C, Supplement 2000.

The ACI is another device for estimating deep lung delivery of a dry powder formulation. The ACI is multi-stage cascade impactor which can be used for determining the particle size (aerodynamic size distribution) of dry powder inhalers (DPI) in accordance with USP 26, Chapter 601, Apparatus 3 (2003).

As described below, the MSLI and the ACI testing devices can be used to determine, inter alia, the fine particle dose (FPD), i.e. the amount of drug, e.g., in micrograms, that is measured in the sections of the testing device which correlates with deep lung delivery and the fine particle fraction (FPF), i.e. the percentage of the metered dose which is measured in the sections of the testing device which correlates with deep lung delivery.

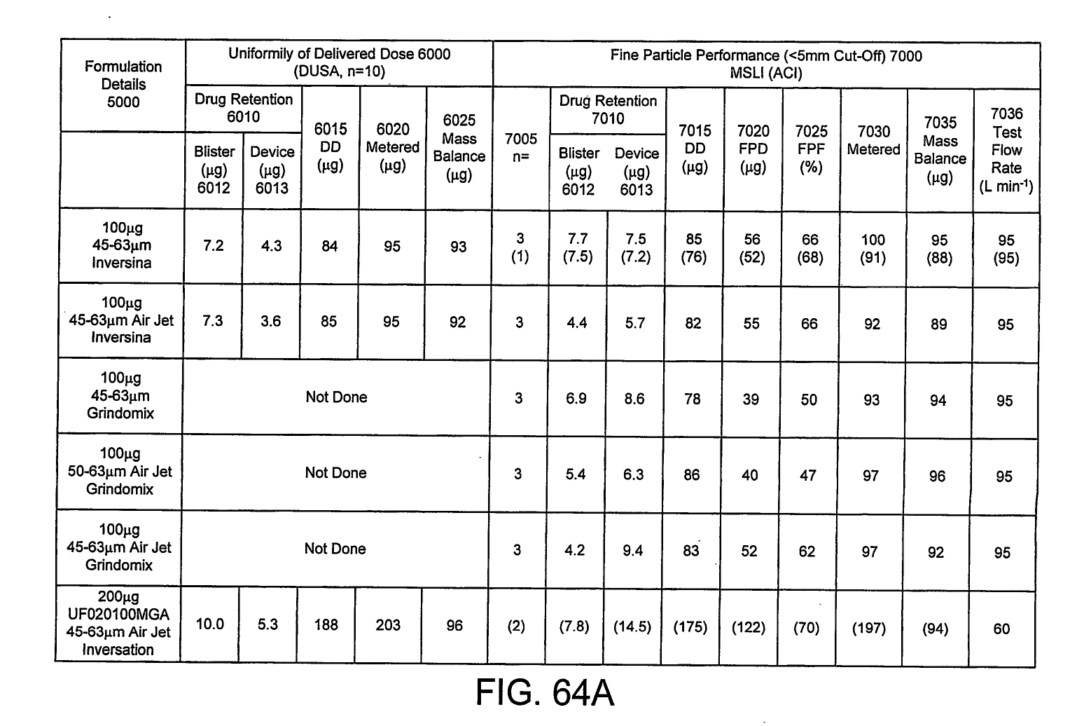

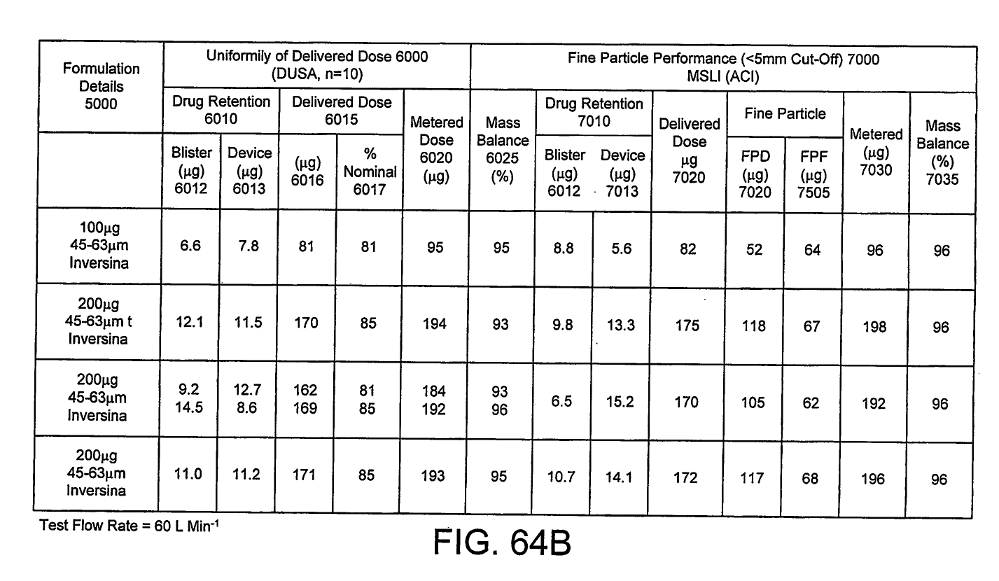

FIGS. 64A and 64B illustrate the results of tests performed on the apomorphine-lactose formulation prepared as follows. Apomorphine hydrochloride was obtained from Macfarlan Smith Ltd, and was micronised according to the following product specification: ≧99.9% by mass <10 μm, based upon a laser diffraction analysis.

Actual typical results of the laser fraction analysis were as follows: d₁₀<1 μm, d₅₀: 1-3 μm; d₉₀<6 μm, wherein d₁₀ d₅₀ d₉₀ refer to the diameter of 10%, 50%, and 90% of the analysed apomorphine hydrochloride. The apomorphine hydrochloride was micronised with nitrogen, (rather than the commonly employed air) to prevent oxidative degradation. The FPD, FPF and MMAD values were generated from the MSLI and ACI data using the Copley Inhaler Data Analysis Software (CITDAS) V1.12. In FIG. 64A, data is shown for six formulations, which are identified in column 5000. FIG. 64B provides data for an additional four formulations. In each Figure, the test data for the formulations is divided into two types: data relating to uniformity of the delivered dose for the formulations (column 6000) and data relating to fine particle size performance of the formulations (column 7000).

Referring to FIG. 64A, the first five formulations listed in column 5000 include 3 mg of the 100 microgram formulation prepared according to the following method ‘B’. A sieved fraction of Respitose SV003 (DMV International Pharma, The Netherlands) lactose is manufactured by passing bulk material through a 63 μm sieve. This material is then sieved through a 45 μm screen and the retained material is collected. The resultant lactose has a volume weighted mean of from about 50 to about 55 μm, a d₁₀ of from about 4 to about 10 μm, a d₅₀ of from about 50 to about 55 μm and a d₉₀ of from about 85 to about 95 μm wherein d₁₀ d₅₀ d₉₀ refer to the diameter of 10%, 50%, and 90% of the analysed lactose.

72.5 grams of this lactose were placed into a metal mixing vessel of a suitable mixer. 5 grams of the micronised apomorphine hydrochloride were then added. An additional 72.5 grams of the lactose were then added to the mixing vessel, and the resultant mixture was tumbled for 15 minutes. The resultant blend was then passed through a 150 μm screen. The screened blend (i.e. the portion of the blend that passed through the screen) was then reblended for 15 minutes.

The mixer used was an Inversina Variable Speed Tumbler Mixer, which is a low shear mixer distributed by Christison Scientific Equipment Ltd of Gateshead, U.K. In other batches, the mixer used was a Retsch Grindomix mixer is a higher shear mixer which is also distributed by Christison Scientific Equipment Ltd. Disaggregation was shown to be sensitive to the intensity of the mixing process but a consistent fine particle fraction (about 60%) was obtained using a low shear mixer equipped with a metal vessel such as the Inversina mixer.

The sixth formulation listed in FIG. 64A includes 3 mg of the 200 microgram formulation prepared according to the following method ‘B’. 70 grams of the lactose described above were placed into a metal mixing vessel of a suitable mixer. 10 grams of the micronised apomorphine hydrochloride were then added. An additional 70 grams of the lactose were then added to the mixing vessel, and the resultant mixture was tumbled for 15 minutes. The resultant blend was then passed through a 150 μm screen. The screened blend (i.e. the portion of the blend that passed through the screen) was then reblended for 15 minutes.

The particle size distribution of the apomorphine-lactose powder, as determined by an Andersen Cascade Impactor (U.S.P 26, Chapter 601, Apparatus 3 (2003)), showed that the drug particles were well dispersed. In particular, the particle size distribution for a 200 μg dose was as follows:

| | | |
|---|---|---|
| Fine particle dose (<5 μm) | 117 | μg |
| Ultrafine particle dose (<2.5 μm) | 80 | μg |
| MMAD (Mass Median Aerodynamic Diameter) | 1.94 | μm |

The first, second, and sixth formulation listings in 5000 of FIG. 64A contain the notation “Inversina” to indicate that the mixer used was the Inversina Mixer, and the third, fourth, and fifth formulation listing contain the notation “Grindomix” to indicate that the mixer used was the Grindomix Mixer. The second and fourth formulations listed also contain the notation “Air Jet” to indicate that for these formulations the lactose was sieved with an Air Jet Sieve which applies a vacuum to the screen sieve apparatus, rather than a conventional screen sieve (which was used for the first third, fifth, and sixth formulations listed). The fifth formulation listed also contains the notation “20-30 μm Extra Fine” to indicate that the lactose for this formulation was screen sieved through 20 μm and 30 μm screens.

In section 6000 of FIG. 64A, the DUSA apparatus described above is used to provide data for the formulations regarding the drug retention in the blister (6012), the drug retention in the inhaler (6013), the delivered dose (6015), the metered dose (6020), and the mass balance percentage (6025). The notation n=10 indicates that the inhaler and DUSA apparatus was fired 10 times for each of the three formulations for which DUSA data is listed. The data listed in section 6000 is an average of the 10 firings.

In section 7000 of FIG. 64B, the fine particle performance is measured with two different devices, the MSLI and the ACI. Data for the ACI, where available, is indicated in parenthesis ( ). In any event, the data provided in section 7000 is for particles having a particle size diameter of less than 5 μm (referred to in this discussion as “fine particles”). As such, column 7012 provides the fine particle drug retention in the blister, column 7013 provides the fine particle drug retention in the inhaler, column 7015 provides the amount of fine particles in the delivered dose, column 7020 provides the FPD for the formulation, column 7025 provides the FPF for the formulation, column 7015 provides the amount of fine particles in the metered dose, column 7035 provides the mass balance percentage for the formulations in the MSLI (ACI) tests, and column 7036 provides the test flow rate for the formulations. Column 7005 indicates that the number of times the inhaler and MSLI (or ACI) apparatus were fired, and the data listed is an average of the “n” firings.

FIG. 64B is similar to FIG. 64A, with similar items bearing identical reference numbers. The first formulation listed in column 5000 include 3 mg of the 100 microgram formulation prepared according to the above method ‘A’, the remaining four formulations include 3 mg of the 200 microgram formulation according to the above method ‘B’, and all of the formulations were made with the Inversina Mixer, and were sieved with 43 and 63 μm screens. The DUSA data in column 6000 was obtained in the same manner as in FIG. 64A, except that n=11. All of the fine particle performance data in section 7000 was obtained using the ACI apparatus with n=2, and a flow rate of 60 L min⁻¹.

As illustrated in FIGS. 64A and 64B, when the formulations were mixed using the low shear Inversina mixer, the fine particle fraction (FPF) ranged from a low of 62% to a high of 70%, and the percent delivered dose ranged from a low of 81% to a high of 94%. The formulations made with the higher shear Grindomix mixer exhibited a fine particle fraction of from 47% to 50% for formulations including the 43-63 μm lactose. The formulation made with the high shear Grindomix mixer and with lactose sieved at 20 and 30 μm exhibited an increased fine particle fraction of 62%.

### EXAMPLE 2

### 400 μg Apomorphine Hydrochloride Capsule for Use in Cyclohaler

Five 400 μg apomorphine hydrochloride capsules were prepared and tested in a Cyclohalet inhaler™ (available from Miat) in an ACI (U.S.P. 26, Chapter 601, Apparatus 3) configured for operation at 100 l.min⁻¹. Each capsule had a fill weight of 25 mg, and included the following components:

| Component | Weight (g) | Weight % (w/w) |
|---|---|---|
| Pharmatose 150 M | 127.725 | 85.15 |
| (DMV Pharma) | | |
| Sorbolac 400 | 12.375 | 8.25 |
| (Meggle Pharma) | | |
| Micronised Leucine | 7.500 | 5.00 |
| Apomorphine Hydrochloride | 2.400 | 1.60 |
| (d₅₀ = 1.453 μm) | | |

In this regard, Pharmatose 150M, available from DMV Pharma, comprises lactose with the following particle size distribution (according to DMV Pharma literature): 100% less than 315 μm, at least 85% less than 150 μm, at least 70% less than 100 μm, and at least 50% less than 45 μm. Sorbolac 400, available from Meggle Pharma comprises lactose with the following particle size distribution (according to Meggle Pharma literature): 100% less than 100 μm, at least 99% less than 63 μm, and at least 96% less than 32 μm.

Preparation of Pre-Blend

The Pharmatose, Sorbolac and leucine were layered in the mixing bowl so that the leucine was sandwiched between the Sorbolac, which in turn was sandwiched between the Pharmatose. The powders were blended for 60 seconds at 2000 rpm using the Retsch Grindomix High Shear Mixer described above. The pre-blend was rested for 1 hour before further use.

Preparation of Final Blend

The apomorphine hydrochloride was sandwiched between the pre-blend in the mixing bowl. Blending was carried out for 10 minutes at 2000 rpm using the Grindomix mixer. The blend was then passed through a 212 μm sieve.

Thereafter, the final blend was placed in capsules, each capsule having a fill weight of 25 mg. The capsules were then placed in a Cyclohaler and tested in an ACI (U.S.P. 26, Chapter 601, Apparatus 3), with the data analysed via the CITDAS described above, providing the following results:

| Delivered Dose (%) | 81% | |
|---|---|---|
| (100 * Delivered Dose/Total Dose) | | |
| % Fine Particle Fraction | 67% | |
| (percent of the delivered dose <5 μm) | | |
| % Fine Particle Dose | 55% | |
| (percent of the total dose <5 μm) | | |
| MMAD | 2.3 | μm |
| Fine Particle Dose | 220 | μg |
| % Ultrafine Particle Dose | 44% | |
| (percent of the total dose <3 μm) | | |
| Ultrafine Particle Dose | 175 | μg |

| | |
|---|---|
| Ultrafine Particle Fraction | 53% |

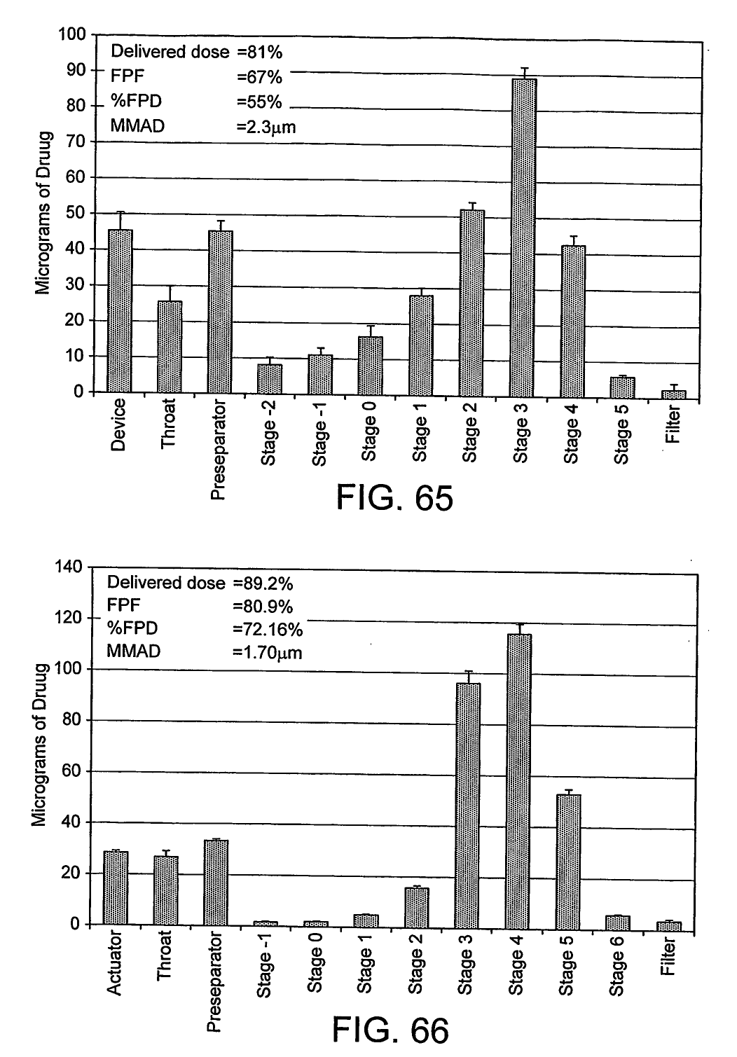

FIG. 65 illustrates the average amount (in micrograms) of drug that was delivered to each of the components of the ACI, and retained in the device. Thus, for example, the ultrafine particle dose can be produced from this data by the CITDAS package.

### EXAMPLE 3

### 400 μg Apomorphine Hydrochloride 2 mg Blister

Five 400% g apomorphine hydrochloride blisters were prepared and tested in the inhaler of Example 1 in an ACI (USP 26, Chapter 601, Apparatus 3) configured for operation at 60 l.min⁻¹. Each blister had a fill weight of 2 mg, and included the following components:

| Component | Weight (g) | Weight % (w/w) |
|---|---|---|
| Respitose 45-63 μm sieve | 120 | 80 |
| Apomorphine Hydrochloride | 30 | 20 |
| (d₅₀ = 1.453 μm) | | |

The apomorphine hydrochloride was sandwiched between the Respitose in the mixing bowl as generally described in methods ‘A’ and ‘B’. The powders were blended for 5 minutes at 2000 rpm using the Grindomix mixer. The blend was then passed through a 212 μm sieve. Thereafter, the blend was placed in blister, each blister having a fill weight of 2 mg. The blisters were then placed in the inhaler of Example 1 and tested in an ACI (U.S.P. 26, Chapter 601, Apparatus 3), with the data analysed via the CITDAS described above, providing the following results:

| Delivered Dose (%) | 89% | |
|---|---|---|
| (100 * Delivered Dose/Total Dose) | | |
| % Fine Particle Fraction | 81% | |
| (percent of the delivered dose < 5 μm) | | |
| % Fine Particle Dose | 72% | |
| (percent of the total dose < 5 μm) | | |
| MMAD | 1.70 | μm |
| Fine Particle Dose | 288 | μg |
| % Ultrafine Particle Dose | 67% | |
| (percent of the total dose < 3 μm) | | |
| Ultrafine Particle Dose | 266 | μg |

| | |
|---|---|
| % Ultrafine Particle Fraction | 75% |
| (percent of the delivered dose < 3 μm) | |

FIG. 66 illustrates the average amount (in micrograms) of drug that was delivered to the components of the ACI, and left in the device. Thus, for example, the ultrafine particle dose can be produced from this data using the CITDAS package.

It should be noted that the MMAD of 1.70 μm generated from the ACI data is remarkably fine, and very close to the median diameter determined by laser light diffraction, for this batch of apomorphine hydrochloride (1.453 μm). This indicates that the inhaler is efficiently reducing the drug to, or close to, its primary particles, rather than agglomerate. This is highly unusual for an inhaler. For example, when the same batch of apomorphine hydrochloride (i.e., in particle size) was delivered with the Cyclohaler of Example 2, a larger MMAD of 2.3 μm was measured, indicating that this formulation and device was not as efficient at eliminating agglomerates.

When compared with the formulation and inhaler of Example 2, the formulation and inhaler of Example 3 also provided a superior delivered dose (89.2% vs. 81%), fine particle fraction (81% vs. 67%), % fine particle dose (72% vs. 55%) and % ultrafine particle dose (67% vs. 44%).

It is also apparent from the above data that the formulation and inhaler of Example 3 produces an ultrafine particle fraction (<3 μm) of more than 700%. While a fine particle fraction (<5 μm) can be considered acceptable for local delivery, it is believed that for systemic delivery, even finer particles are needed, because the drug must reach the alveoli to be absorbed into the bloodstream. As such an ultrafine particle fraction in excess of 70% is particularly advantageous.

### EXAMPLE 4

### Preparation of MechanoFused Formulation for Use in Passive Device

20 g of a mix comprising 20% micronised clomipramine, 78% Sorbolac 400 lactose and 2% magnesium stearate were weighed into the Hosokawa AMS-MINI MechanoFusion system via a funnel attached to the largest port in the lid with the equipment running at 3.5%. The port was sealed and the cooling water switched on. The equipment was run at 20% for 5 minutes followed by 80% for 10 minutes. The equipment was switched off, dismantled and the resulting formulation recovered mechanically.

20 mg of the collected powder formulation was filled into size 3 capsules and fired from a Miat Monhaler into an NGI. The FPF measured was greater than 70%.

In light of the foregoing data and examples, one can see that excellent performance of the powder formulations according to the present invention (defined in terms of FPF(ED) and FPF(MD) at 5 μm=, 3 μm and 2 μm) is achieved in vitro. What is more, this high performance also leads to excellent in vivo performance, including achieving faster peak blood levels than alternative systems. Indeed, peak blood levels may be achieved within 1 to 10 minutes from administration when using the present invention. This, in turn, leads to a faster onset of the clinical effect than is observed with alternative systems. Indeed, the onset may be 2, 3, 5, or even 10 times faster when using the present invention.

Another very important advantage of the system of the present invention is the consistency of the high performance. The data set out above shows that the excellent performance is repeatable with very low variability. One of the many benefits of such consistency is that it can also lead to reduction in adverse side effects experienced, as it will allow one to administer a smaller total dose than is possible when relying upon conventional inhaler efficiency or other routes of administration. In particular, it allows one to target specific dosing windows wherein the therapeutic effect is maximised whilst causing the minimum side effects.

The system of the present invention is extremely flexible and therefore has a vast number of applications.

The formulations may be administered using active or passive devices, as it has been identified how to tailor the formulation to the device used to dispense it, thereby overcoming some of the perceived disadvantages of passive devices where high performance is sought.

The size of the doses can vary from micrograms to tens of milligrams. The fact that dense particles may be used, in contrast to conventional thinking, means that larger doses can be administered without needing to administer large volumes of powder and the problems associated therewith.

The dry powder formulations may be pre-metered and kept in foil blisters which offer chemical and physical protection whilst not being detrimental to the overall performance. Indeed, the formulations thus packaged tend to be stable over long periods of time, which is very beneficial, especially from a commercial and economic point of view.

The milling methods used in the present invention for preparing the fine particles of active agent are simple and cheap compared to the complex previous attempts to engineer particles, providing practical as well as cost benefits. In addition, the spray drying methods provided herein are also capable of being carried out on a large scale, again providing practical and cost benefits.

A further benefit associated with the present invention is that the powder process step may be dry, which means that it does not have to involve organic solvents. Such organic solvents are common to many of the known approaches to powder processing.

In addition, the active agents used in the present invention may be small molecules, proteins, carbohydrates or mixtures thereof.

Finally, the particles prepared as described herein are not “low density” particles, as tend to be favoured in the prior art. Rather, the jet milled and spray dried particles are made using simple processes. Previously, those skilled in the art have only reported high performance in connection with powder particles that have been prepared using fancy processing techniques such as complex spray drying, which result in low density particles. As explained above, surprisingly it is advantageous not to produce severely dimpled or wrinkled particles as these can yield low density powders, with very high voidage between particles. Such powders occupy a large volume relative to their mass as a consequence of this form, and can result in packaging problems, i.e., require much larger blisters or capsules are required to hold a given mass of powder.

Advantageously, the powders prepared according to the present invention have a tapped density of at least 0.1 g/cc, at least 0.2 g/cc, at least 0.3 g/cc, at least 0.4 g/cc or at least 0.5 g/cc.

1. A dry powder inhaler device comprising a dry powder formulation comprising a pharmaceutically active agent, wherein upon actuation of the device, a dosing efficiency at 5 μm of at least 70% is achieved.

2. A device as claimed in claim 1, wherein a dosing efficiency at 3 μm of at least 60% is achieved

3. A device as claimed in claim 1, wherein a dosing efficiency at 2 μm of preferably at least 40% is achieved.

4. A device as claimed in claim 1, wherein the dry powder composition was prepared using a method comprising co-spray drying the pharmaceutically active agent with a force control agent.

5. A device as claimed in claim 4, wherein the force control agent is an amino acid, a phospholipid or a metal stearate, and is preferably leucine.

6. A device as claimed in claim 4, wherein the active agent is spray dried using a spray drier comprising a means for producing droplets moving at a controlled velocity and of a predetermined size.

7. A device as claimed in claim 6, wherein the spray drier comprises an ultrasonic nebuliser.

8. A device as claimed in claim 4, wherein the method comprises adjusting the moisture content of the spray dried particles.

9. A device as claimed in claim 1, wherein composite active particles for use in the pharmaceutical composition are prepared using a method comprising jet milling active particles in the presence of particles of additive material.

10. A device as claimed in claim 9, wherein the additive material comprises an amino acid, a metal stearate or a phospholipid.

11. A device as claimed in claim 10, wherein the additive material comprises one or more of leucine, isoleucine, lysine, valine, methionine, phenylalanine, and preferably leucine.

12. A device as claimed in claim 1, wherein the device is an active device.

13. A device as claimed in claim 1, wherein the device is a passive device.

14. A device as claimed in claim 1, wherein the dry powder formulation is in pre-metered doses stored in one or more foil blisters.

15. A device as claimed in claim 1, wherein the dry powder formulation has a fine particle dose of the emitted dose of at least 70%.

16. A device as claimed in claim 15, wherein the fine particle dose is at least 80%.

17. A device as claimed claim 1 in, wherein the dry powder formulation has a fine particle dose of the metered dose of at least 65%.

18. A device as claimed in claim 16, wherein the fine particle dose is at least 75%.

19. A device as claimed in claim 1, wherein the dry powder formulation dispensed upon actuation produces a peak blood plasma level within 1 to 20 minutes of pulmonary inhalation.

20. A device as claimed in claim 19, wherein the peak blood plasma level within 1 to 10 minutes of pulmonary inhalation.

21. A device as claimed in claim 1, wherein the dry powder formulation dispensed upon actuation produces the pharmacodynamic effect within 15 minutes of pulmonary inhalation.

22. A device as claimed in claim 21, wherein the effect is produced within 10 minutes of pulmonary inhalation.

23. A device as claimed in claim 21, wherein the effect is produced within 5 minutes of pulmonary inhalation.

24. A device as claimed in claim 1, wherein the onset of the effect of the pharmaceutically active agent following pulmonary inhalation is twice as fast as the onset of the effect when the agent is administered via the oral route.

25. A device as claimed in claim 24, wherein the onset of the effect is three times faster than that achieved by administration via the oral route.

26. A device as claimed in claim 24, wherein the onset of the effect is five times faster than that achieved by administration via the oral route.

27. A device as claimed in claim 24, wherein the onset of the effect is eight times faster than that achieved by administration via the oral route

28. A device as claimed in claim 1, wherein the effect of the dry powder formulation following pulmonary inhalation is such that the dose of the pharmaceutically active agent is reduced by at least 50% compared to the dose required to have the same effect when administered via the oral route.

29. A device as claimed in claim 28, wherein the dose is reduced by at least 70%.

30. A device as claimed in claim 28, wherein the dose is reduced by at least 80%.

31. A device as claimed in claim 28, wherein the dose is reduced by at least 90%.

32. A device as claimed in claim 1, wherein the administration of the dry powder formulation by pulmonary inhalation does not cause the adverse side effects normally associated with the administration of the pharmaceutically active agent via other routes.

33. A device as claimed in claim 1, wherein the dry powder formulation is produced by a micronisation process.

34. A device as claimed in claim 1, wherein the dry powder formulation has a tapped density of more than 0.1 g/cc.

35. A device as claimed in claim 34, wherein the formulation has a tapped density of more than 0.2 g/cc.

36. A device as claimed in claim 34, wherein the formulation has a tapped density of more than 0.5 g/cc.

37. A device as claimed in claim 1, wherein the pharmaceutically active agent has a systemic effect following administration by pulmonary inhalation.

38. A device as claimed in claim 1, wherein the pharmaceutically active agent is a small molecule or a carbohydrate.

39. A device as claimed in claim 1, wherein the dry powder formulation is processed without the use of an organic solvent.

40. A device as claimed in claim 1, wherein the dry powder formulation is dry processed in the absence of any solvent.
